(19) United States
(12) Patent Application Publication
(10) Pub. No.: US 2025/0255989 A1
(43) Pub. Date: Aug. 14, 2025

(54) ADENO-ASSOCIATED VIRUS COMPOSITIONS HAVING INCREASED BRAIN ENRICHMENT AND/OR HEART ENRICHMENT

(71) Applicant: CAPSIDA, INC, Thousand Oaks, CA (US)

(72) Inventors: Xiaojing Shi, Thousand Oaks, CA (US); Brandon G. Wheeler, Thousand Oaks, CA (US); Troy E. Sandberg, Thousand Oaks, CA (US); Nicholas S. Goeden, Thousand Oaks, CA (US); Nicholas C. Flytzanis, Thousand Oaks, CA (US)

(21) Appl. No.: 18/867,263

(22) PCT Filed: Jun. 6, 2023
(86) PCT No.: PCT/US2023/067973

§ 371 Date: Nov. 19, 2024

Related U.S. Application Data
(60) Provisional application No. 63/352,816, filed on Jun. 16, 2022.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143; C12N 2750/14145

(57) ABSTRACT

Described herein are compositions and kits comprising recombinant adeno-associated viruses (rAAVs) with increased transduction enrichment in the brain, heart, or brain and heart. The rAAV compositions described herein encapsidate a transgene, such as a therapeutic nucleic acid. Gene therapy using the rAAVs is described. Also described are methods of treating brain-related and heart-related diseases and conditions.

### BACKGROUND

Recombinant adeno-associated viruses (rAAVs) are widely used as vectors for gene delivery in therapeutic applications because of their ability to transduce both dividing and non-dividing cells, their long-term persistence as episomal DNA in infected cells, and their low immunogenicity. These characteristics make them appealing for applications in therapeutic applications, such as gene therapy. However, there is a need to significantly improve the performance of existing AAV serotypes to selectively and efficiently express in distinct cell-types, upon systemic delivery to a subject. This need is especially acute when the AAV must be expressed in the brain or heart.

### SUMMARY OF THE INVENTION

Disclosed herein are rAAVs with peptide insertions and substitutions engineered into the capsid structure through iterative rounds of selection in non-human primates (NHPs), yielding variants having increased transduction when measured in the brain, heart, or brain and heart relative to the wild type rAAV on which the variant is based.

The present invention provides rAAVs with widespread transduction to the brain, heart, or brain and heart. Following IV injection, unmodified rAAVs such as those derived from AAV9 (SEQ ID NO: 1) may not have sufficient tissue enrichment to treat many human diseases by delivery of an AAV cargo Directed evolution of AAV9 as described herein has provided modified rAAVs that exhibit increased viral tissue enrichment in the brain. Accordingly, engineered rAAVs described herein are particularly useful in delivering DNA cargo to brain tissue and/or heart tissue.

The present invention provides a mutant Adeno-Associated Virus (AAV) capsid protein comprising at least 95% sequence identity to the wild-type AAV capsid protein, wherein the mutant AAV capsid protein comprises a sequence selected from the group consisting of SEQ ID NO: 2-1496.

The mutant AAV capsid protein may comprise a 7-mer amino acid insertion sequence located between residues 588 and 589 in relation to the wild-type AAV capsid protein. The mutant AAV capsid protein may comprise at least a 95% sequence identity to the wild-type Adeno-Associated Virus serotype 9 (AAV9) capsid of SEQ ID NO: 1. In the AAV9 capsid, the amino acids immediately preceding the 7-mer amino acid insertion sequence are -AQ-. The amino acids immediately following the 7-mer amino acid insertion sequence are also typically -AQ-.

In aspects of the invention, 60 copies of the AAV capsid protein may be assembled into the AAV capsid. The AAV capsid protein may present in VP1, VP2, and VP3 of the mutant AAV capsid.

The mutant AAV capsid protein may comprise a sequence selected from the group consisting of SEQ ID NO: 2-898. Advantageously, the AAV capsid protein may be characterized with increased transduction enrichment relative to the wild-type AAV9 when measured in brain tissue in a subject when delivered to the subject systemically. In aspects of the invention, the mutant AAV capsid protein comprises a sequence selected from the group consisting of SEQ ID NO: 2-594.

The mutant AAV capsid protein may comprise a sequence selected from the group consisting of SEQ ID NO: 595-1496. Advantageously, the mutant AAV capsid protein may be characterized by an increased transduction enrichment relative to the wild-type AAV9 when measured in heart tissue in a subject when delivered to the subject systemically. In aspects of the invention, the mutant AAV capsid protein comprises a sequence selected from the group consisting of SEQ ID NO: 899-1496.

The mutant AAV capsid protein may comprise a sequence selected from the group consisting of SEQ ID NO: 595-898. The mutant AAV capsid protein may be characterized by an increased transduction enrichment relative to the wild-type AAV9 when measured in brain tissue and heart tissue in a subject when delivered to the subject systemically.

In aspects of the invention the AAV capsid protein is integrated into an AAV capsid. The AAV capsid is chimeric. The AAV capsid may be isolated and purified.

Aspects of the invention further provide a composition comprising the AAV capsid of the invention, wherein the AAV capsid comprises an AAV capsid protein comprising at least 95% sequence identity to the wild-type AAV capsid protein, wherein the mutant AAV capsid protein comprises a sequence selected from the group consisting of SEQ ID NO: 2-1496. The composition may be formulated for systematic administration to a subject. Systematic administration of the composition may result in expression of a gene product delivered by the AAV capsid. Administration of the composition results in increased transduction enrichment relative to the wild-type AAV9 when measured in heart and/or brain.

Aspects of the invention further provide a method of treating a disease or condition in a subject comprising administering a therapeutically effective amount of the composition of the invention. For example, provided is a composition for use in treating a disease or condition in a subject, the use comprising administering a therapeutically effective amount of a composition of the invention. Advantageously, because the compositions of the invention comprise AAV capsid proteins that are direct engineered AAVs to brain tissue, heart tissue, or brain and heart tissues 10 specifically, the compositions provide effective methods and uses for treating diseases associated with the brain, heart, or brain and heart.

TABLE 1
Brain Targeting sequences

| SEQ ID | AA sequence |
|---|---|
| NO: | (#587-597) |
| 2 | AQAAATAAQAQ |
| 3 | AQAALPPRSAQ |
| 4 | AQAASSGHSAQ |
| 5 | AQAESRTSAAQ |
| 6 | AQAFSVSPDAQ |
| 7 | AQAGGHSNNAQ |
| 8 | AQAGPSNAMAQ |
| 9 | AQAGSIRDDAQ |
| 10 | AQAHGDGSLAQ |
| 11 | AQAHSSGVGAQ |
| 12 | AQAHTHTLSAQ |
| 13 | AQAIAGYASAQ |
| 14 | AQAISNTTVAQ |
| 15 | AQALARDHSAQ |
| 16 | AQALRGSLCAQ |
| 17 | AQAMDRTNMAQ |
| 18 | AQAMQTRNPAQ |
| 19 | AQAMSSLTAAQ |
| 20 | AQAPGALMTAQ |
| 21 | AQAPREIAIAQ |
| 22 | AQAQADRPDAQ |
| 23 | AQAQGNLAFAQ |
| 24 | AQARSNEMTAQ |
| 25 | AQASGHAPTAQ |
| 26 | AQASGSNAHAQ |
| 27 | AQASLAVTSAQ |
| 28 | AQASRDSGSAQ |
| 29 | AQASRSESYAQ |
| 30 | AQASTRVVNAQ |
| 31 | AQATSDRPTAQ |
| 32 | AQATVTHKDAQ |
| 33 | AQAVGTMAPAQ |
| 34 | AQDASRVLAAQ |
| 35 | AQDMTGLVIAQ |
| 36 | AQDNRTRTNAQ |
| 37 | AQDPSRSASAQ |
| 38 | AQDPTTRNEAQ |
| 39 | AQDRSNITLAQ |
| 40 | AQDSAQNRGAQ |
| 41 | AQDSGPSKAAQ |
| 42 | AQDSIQYSFAQ |
| 43 | AQDSREQGRAQ |
| 44 | AQDSRLGVTAQ |
| 45 | AQDSRQPPNAQ |
| 46 | AQDSRVPSQAQ |
| 47 | AQDSVNLSSAQ |
| 48 | AQDVRHGGTAQ |
| 49 | AQDVRSNTSAQ |
| 50 | AQDVSVSAFAQ |
| 51 | AQDYAYRTGAQ |
| 52 | AQEHTRGETAQ |
| 53 | AQEITHSHSAQ |
| 54 | AQENHTENKAQ |
| 55 | AQENRVANKAQ |
| 56 | AQEPERFMFAQ |
| 57 | AQEPRLATDAQ |
| 58 | AQERTASLHAQ |
| 59 | AQESNRHPYAQ |
| 60 | AQESSHTGGAQ |
| 61 | AQESVGRGEAQ |
| 62 | AQEVGGGSKAQ |
| 63 | AQFALPVPSAQ |
| 64 | AQFGSVQDRAQ |
| 65 | AQFGTGGVPAQ |
| 66 | AQFGTMGDKAQ |
| 67 | AQFGVTAGSAQ |
| 68 | AQFINTVPVAQ |
| 69 | AQFLVGGTVAQ |
| 70 | AQFSAFLPAAQ |
| 71 | AQFSGMSNSAQ |
| 72 | AQFVKIAKSAQ |
| 73 | AQFVTLVTSAQ |
| 74 | AQGADARLHAQ |
| 75 | AQGDAARSGAQ |
| 76 | AQGGGSHSSAQ |
| 77 | AQGHQHVTEAQ |
| 78 | AQGHSETRLAQ |
| 79 | AQGIGHMNDAQ |
| 80 | AQGLALTLNAQ |
| 81 | AQGLAVASPAQ |
| 82 | AQGLSSPLAAQ |
| 83 | AQGLVLSSTAQ |
| 84 | AQGLVSSSVAQ |
| 85 | AQGLYNIRSAQ |
| 86 | AQGNAVSVAAQ |
| 87 | AQGNHTRHAAQ |
| 88 | AQGNLTRLTAQ |
| 89 | AQGNNTRAHAQ |
| 90 | AQGNNTTSRAQ |
| 91 | AQGNQSNAAAQ |
| 92 | AQGNRTRAPAQ |
| 93 | AQGNRTTNDAQ |
| 94 | AQGNTTVNQAQ |
| 95 | AQGSDSRNSAQ |
| 96 | AQGSFTQFSAQ |
| 97 | AQGSGASVLAQ |
| 98 | AQGSGISSHAQ |
| 99 | AQGSGTPGHAQ |
| 100 | AQGSLTVLKAQ |
| 101 | AQGSMMHKDAQ |
| 102 | AQGSSRNEVAQ |
| 103 | AQGTGSERLAQ |
| 104 | AQGTQDRQLAQ |
| 105 | AQGTTLSSSAQ |
| 106 | AQGTTSHHLAQ |
| 107 | AQGVGLPTTAQ |
| 108 | AQGVGSSLSAQ |
| 109 | AQGVHASTQAQ |
| 110 | AQGVHGSNAAQ |
| 111 | AQGVISRPDAQ |
| 112 | AQGVSAVAAAQ |
| 113 | AQGVTSVVAAQ |
| 114 | AQHAGNITIAQ |
| 115 | AQHGSRSDYAQ |
| 116 | AQHGVTTQSAQ |
| 117 | AQHGVVSSSAQ |
| 118 | AQHHSPQSSAQ |
| 119 | AQHISLHQQAQ |
| 120 | AQHISSVQPAQ |
| 121 | AQHLGTVSAAQ |
| 122 | AQHMSGVNAAQ |
| 123 | AQHNVMSSFAQ |
| 124 | AQHNVSLANAQ |
| 125 | AQHQLHHGAAQ |
| 126 | AQHSSGTSGAQ |
| 127 | AQHSSSNIIAQ |
| 128 | AQHSTKAEVAQ |
| 129 | AQHTANLLDAQ |
| 130 | AQHTGASLTAQ |
| 131 | AQHTGPLSNAQ |
| 132 | AQHTLPIMSAQ |
| 133 | AQHTSTREMAQ |
| 134 | AQHTSVRIDAQ |
| 135 | AQHTTSHMEAQ |
| 136 | AQHYVSVPTAQ |
| 137 | AQIAGRNQSAQ |
| 138 | AQIAHLSSVAQ |
| 139 | AQIASSPLSAQ |
| 140 | AQIASSSLHAQ |
| 141 | AQIGGLTTPAQ |
| 142 | AQIGSLTGAAQ |
| 143 | AQIGSMNEKAQ |
| 144 | AQIGSSMPNAQ |
| 145 | AQILATGGSAQ |
| 146 | AQILDRSNMAQ |
| 147 | AQILRDGHQAQ |
| 148 | AQILSEHRVAQ |
| 149 | AQILTRSTFAQ |
| 150 | AQILVTSTSAQ |
| 151 | AQIMTSTLPAQ |
| 152 | AQINRETSFAQ |
| 153 | AQINSAGLLAQ |
| 154 | AQISAASTSAQ |
| 155 | AQISAGALIAQ |
| 156 | AQISLTNSNAQ |
| 157 | AQISSSRNGAQ |
| 158 | AQISTGKLGAQ |
| 159 | AQISTRESIAQ |
| 160 | AQITGQMSAAQ |
| 161 | AQITGQMSVAQ |
| 162 | AQITSSPTSAQ |
| 163 | AQITSSVMQAQ |
| 164 | AQITTHPTTAQ |
| 165 | AQIVAGHRDAQ |
| 166 | AQIVDTRNPAQ |
| 167 | AQIVGKPMEAQ |
| 168 | AQIVLPSTYAQ |
| 169 | AQIVSGSLGAQ |
| 170 | AQIYSSSVSAQ |
| 171 | AQKDQNRESAQ |
| 172 | AQKDSHSYNAQ |
| 173 | AQKEQQAVSAQ |
| 174 | AQKHNIPNVAQ |
| 175 | AQKPTVGFAAQ |
| 176 | AQKQAGTSVAQ |
| 177 | AQKQHSDHPAQ |
| 178 | AQKSQTNDVAQ |
| 179 | AQKSSLRFYAQ |
| 180 | AQKTSSSTVAQ |
| 181 | AQLAHQTRDAQ |
| 182 | AQLAQAASAAQ |
| 183 | AQLARTTTDAQ |
| 184 | AQLASASSVAQ |
| 185 | AQLASSTLMAQ |
| 186 | AQLDRSGSTAQ |
| 187 | AQLDTSTALAQ |
| 188 | AQLGAGPHVAQ |
| 189 | AQLGGSYGSAQ |
| 190 | AQLGSLANTAQ |
| 191 | AQLGTNRMSAQ |
| 192 | AQLGVSSVEAQ |
| 193 | AQLHNTVVSAQ |
| 194 | AQLHSPASNAQ |
| 195 | AQLHSSPSTAQ |
| 196 | AQLILTTSEAQ |
| 197 | AQLKDQGSSAQ |
| 198 | AQLLDTHYRAQ |
| 199 | AQLLDTLMQAQ |
| 200 | AQLLITAVQAQ |
| 201 | AQLLLTPQLAQ |
| 202 | AQLLRSTLPAQ |
| 203 | AQLLSATTHAQ |
| 204 | AQLLSSSNSAQ |
| 205 | AQLMSILTMAQ |
| 206 | AQLMSSVSAAQ |
| 207 | AQLNHSVSGAQ |
| 208 | AQLNHTSPGAQ |
| 209 | AQLNPQGTSAQ |
| 210 | AQLNSDKSSAQ |
| 211 | AQLNSTRTYAQ |
| 212 | AQLNTAQSTAQ |
| 213 | AQLNTGSNAAQ |
| 214 | AQLNVSTTSAQ |
| 215 | AQLPASDISAQ |
| 216 | AQLPRDKWEAQ |
| 217 | AQLPSETKFAQ |
| 218 | AQLQSNRSDAQ |
| 219 | AQLQSRSFGAQ |
| 220 | AQLQTSLSLAQ |
| 221 | AQLRNSESTAQ |
| 222 | AQLRNSSELAQ |
| 223 | AQLRTDSLEAQ |
| 224 | AQLSDHNLKAQ |
| 225 | AQLSGFNVLAQ |
| 226 | AQLSGHFSFAQ |
| 227 | AQLSGMSGNAQ |
| 228 | AQLSGQSMDAQ |
| 229 | AQLSGQVTMAQ |
| 230 | AQLSNESRSAQ |
| 231 | AQLSNITNSAQ |
| 232 | AQLSQASSAAQ |
| 233 | AQLSQSGKEAQ |
| 234 | AQLSRSFVPAQ |
| 235 | AQLSRSSSPAQ |
| 236 | AQLSSANLQAQ |
| 237 | AQLSSSITQAQ |
| 238 | AQLSSSVVNAQ |
| 239 | AQLSSTLTSAQ |
| 240 | AQLSSTYANAQ |
| 241 | AQLSTDASQAQ |
| 242 | AQLSTISSHAQ |
| 243 | AQLSTSGPSAQ |
| 244 | AQLSVAQGSAQ |
| 245 | AQLSVLSQIAQ |
| 246 | AQLTGNQSVAQ |
| 247 | AQLTHTIPSAQ |
| 248 | AQLTNVIMGAQ |
| 249 | AQLTSLPQVAQ |
| 250 | AQLTTPSAAAQ |
| 251 | AQLTTSSTLAQ |
| 252 | AQLTTTHSQAQ |
| 253 | AQLVIMGMSAQ |
| 254 | AQLVSSESRAQ |
| 255 | AQLVTAGTAAQ |
| 256 | AQMDTHNLVAQ |
| 257 | AQMDYSTSPAQ |
| 258 | AQMGSSGQMAQ |
| 259 | AQMGTAHTIAQ |
| 260 | AQMGTHVVTAQ |
| 261 | AQMHDGRLVAQ |
| 262 | AQMLAKTATAQ |
| 263 | AQMLSSTMRAQ |
| 264 | AQMLSTTSGAQ |
| 265 | AQMLVKHAGAQ |
| 266 | AQMMSSTNAAQ |
| 267 | AQMNHNQQHAQ |
| 268 | AQMNHTSNKAQ |
| 269 | AQMNNKSPGAQ |
| 270 | AQMNNSTRPAQ |
| 271 | AQMNSSSHSAQ |
| 272 | AQMSGNSMAAQ |
| 273 | AQMSTSYSSAQ |
| 274 | AQMYLENNSAQ |
| 275 | AQNAGYSAAAQ |
| 276 | AQNANHAMPAQ |
| 277 | AQNAQGTHVAQ |
| 278 | AQNASEKSSAQ |
| 279 | AQNASNSTTAQ |
| 280 | AQNGLRADTAQ |
| 281 | AQNGRTGTGAQ |
| 282 | AQNGRTTTVAQ |
| 283 | AQNGSITGTAQ |
| 284 | AQNGSRGAAAQ |
| 285 | AQNHEQYQWAQ |
| 286 | AQNHSGTTSAQ |
| 287 | AQNHTGVSPAQ |
| 288 | AQNHVGGATAQ |
| 289 | AQNIQSLSTAQ |
| 290 | AQNLGLISVAQ |
| 291 | AQNLSLTHEAQ |
| 292 | AQNMSSNKDAQ |
| 293 | AQNMTSMQSAQ |
| 294 | AQNPTRSLSAQ |
| 295 | AQNQCQSHVAQ |
| 296 | AQNQFTATEAQ |
| 297 | AQNQLGNSAAQ |
| 298 | AQNQQNKANAQ |
| 299 | AQNRNEVHSAQ |
| 300 | AQNRSDSAMAQ |
| 301 | AQNSAFTAQAQ |
| 302 | AQNSFSPATAQ |
| 303 | AQNSGNYQIAQ |
| 304 | AQNSHGHALAQ |
| 305 | AQNSPGYTAAQ |
| 306 | AQNSSAATGAQ |
| 307 | AQNSSGHGVAQ |
| 308 | AQNSSSGSAAQ |
| 309 | AQNSSSSPGAQ |
| 310 | AQNSTSISHAQ |
| 311 | AQNTLSNISAQ |
| 312 | AQNTQTHSSAQ |
| 313 | AQNTTQPSNAQ |
| 314 | AQNVMSSGAAQ |
| 315 | AQNVQSSNLAQ |
| 316 | AQNYCGKECAQ |
| 317 | AQPATGTSGAQ |
| 318 | AQPAVGSAGAQ |
| 319 | AQPIGGSNMAQ |
| 320 | AQPIHQTGVAQ |
| 321 | AQPLQTSNAAQ |
| 322 | AQPLTGQTNAQ |
| 323 | AQPLYNQTPAQ |
| 324 | AQPPKPPSIAQ |
| 325 | AQPQPHRLLAQ |
| 326 | AQPQRSQHIAQ |
| 327 | AQPQSNVSTAQ |
| 328 | AQPSAEQAQAQ |
| 329 | AQPSGAYGEAQ |
| 330 | AQPSGQKIDAQ |
| 331 | AQPSGSHSIAQ |
| 332 | AQPSGTNIGAQ |
| 333 | AQPSNALAGAQ |
| 334 | AQPSNPQSYAQ |
| 335 | AQPSPSTMRAQ |
| 336 | AQPSRDVTPAQ |
| 337 | AQPSSYSDSAQ |
| 338 | AQPVAAHVRAQ |
| 339 | AQPVHGSTDAQ |
| 340 | AQQAAVSGGAQ |
| 341 | AQQATHVALAQ |
| 342 | AQQDSTKALAQ |
| 343 | AQQIHGPMQAQ |
| 344 | AQQLLVSVTAQ |
| 345 | AQQLSGSLMAQ |
| 346 | AQQLSHEKSAQ |
| 347 | AQQNNTHGGAQ |
| 348 | AQQNSGPLHAQ |
| 349 | AQQRTDQQTAQ |
| 350 | AQQVAHTQIAQ |
| 351 | AQQVGKSSAAQ |
| 352 | AQQVISTGSAQ |
| 353 | AQQVRTNTSAQ |
| 354 | AQRADRVQEAQ |
| 355 | AQRDKMVFMAQ |
| 356 | AQRDSVTDSAQ |
| 357 | AQRESRVSQAQ |
| 358 | AQRGHGQEIAQ |
| 359 | AQRGLSAVNAQ |
| 360 | AQRIGSSAIAQ |
| 361 | AQRISSETSAQ |
| 362 | AQRLDIHMCAQ |
| 363 | AQRNTSNQEAQ |
| 364 | AQRSNNCIGAQ |
| 365 | AQRSNVDGLAQ |
| 366 | AQRSSLHTSAQ |
| 367 | AQRSTESLHAQ |
| 368 | AQRTAANHEAQ |
| 369 | AQRTDGTSIAQ |
| 370 | AQRTHSAMPAQ |
| 371 | AQRTSGPAAAQ |
| 372 | AQSAPNRTDAQ |
| 373 | AQSAQSAIQAQ |
| 374 | AQSARGMSGAQ |
| 375 | AQSASRDPAAQ |
| 376 | AQSDHSVRDAQ |
| 377 | AQSDKNLQQAQ |
| 378 | AQSDLICCPAQ |
| 379 | AQSERSYPSAQ |
| 380 | AQSFSGQSLAQ |
| 381 | AQSFSNVSPAQ |
| 382 | AQSGLNTLAAQ |
| 383 | AQSGNHNPAAQ |
| 384 | AQSGPVQNQAQ |
| 385 | AQSGSSHERAQ |
| 386 | AQSGSVMSSAQ |
| 387 | AQSHIVSSSAQ |
| 388 | AQSHLSSQVAQ |
| 389 | AQSHNSAFEAQ |
| 390 | AQSIARDLAAQ |
| 391 | AQSIGSSYPAQ |
| 392 | AQSIKQNEGAQ |
| 393 | AQSITQGSAAQ |
| 394 | AQSKNDTSMAQ |
| 395 | AQSLAGTLSAQ |
| 396 | AQSLANSQTAQ |
| 397 | AQSLGAQLFAQ |
| 398 | AQSLHLSSSAQ |
| 399 | AQSLSHTRPAQ |
| 400 | AQSLSQTVGAQ |
| 401 | AQSLSSTGGAQ |
| 402 | AQSLVSLKEAQ |
| 403 | AQSLVVTTPAQ |
| 404 | AQSMGSSNWAQ |
| 405 | AQSMISSNRAQ |
| 406 | AQSMMSLDQAQ |
| 407 | AQSMPQPLVAQ |
| 408 | AQSMSSNLTAQ |
| 409 | AQSMTLSNYAQ |
| 410 | AQSNATHRFAQ |
| 411 | AQSNHTKPIAQ |
| 412 | AQSNQTRHVAQ |
| 413 | AQSPKPPSTAQ |
| 414 | AQSQDTASKAQ |
| 415 | AQSQIAEPAAQ |
| 416 | AQSQKAPDIAQ |
| 417 | AQSQLSPSNAQ |
| 418 | AQSQMSSAGAQ |
| 419 | AQSQSSSQTAQ |
| 420 | AQSRESPSTAQ |
| 421 | AQSRLLESSAQ |
| 422 | AQSRNQPAEAQ |
| 423 | AQSRSSDAFAQ |
| 424 | AQSRTDLLSAQ |
| 425 | AQSRTSNQAAQ |
| 426 | AQSRVEEMNAQ |
| 427 | AQSRVNTLDAQ |
| 428 | AQSSAVLLNAQ |
| 429 | AQSSDRVSSAQ |
| 430 | AQSSGHSSEAQ |
| 431 | AQSSGLRLGAQ |
| 432 | AQSSGSREVAQ |
| 433 | AQSSHDTLQAQ |
| 434 | AQSSLSVLWAQ |
| 435 | AQSSLSVVPAQ |
| 436 | AQSSMWHKDAQ |
| 437 | AQSSQSFGYAQ |
| 438 | AQSSREVVTAQ |
| 439 | AQSSRGETPAQ |
| 440 | AQSSSQSSDAQ |
| 441 | AQSSTQHEKAQ |
| 442 | AQSSVSRTSAQ |
| 443 | AQSSYGYRSAQ |
| 444 | AQSTALTTGAQ |
| 445 | AQSTATSPAAQ |
| 446 | AQSTGQGFTAQ |
| 447 | AQSTGQGINAQ |
| 448 | AQSTGTSGQAQ |
| 449 | AQSTISTGSAQ |
| 450 | AQSTKQNVNAQ |
| 451 | AQSTMAIGSAQ |
| 452 | AQSTMGALKAQ |
| 453 | AQSTNRAEQAQ |
| 454 | AQSTSALGPAQ |
| 455 | AQSTSDYSRAQ |
| 456 | AQSTSPTYVAQ |
| 457 | AQSTSSITVAQ |
| 458 | AQSTSSPTSAQ |
| 459 | AQSTSYASIAQ |
| 460 | AQSTTRVVDAQ |
| 461 | AQSVAPIANAQ |
| 462 | AQSVGNGNIAQ |
| 463 | AQSVRSGFKAQ |
| 464 | AQSVSDGGFAQ |
| 465 | AQSVSSNIVAQ |
| 466 | AQSVSTAAQAQ |
| 467 | AQTAHNVEQAQ |
| 468 | AQTAPQSMDAQ |
| 469 | AQTDRGQVQAQ |
| 470 | AQTDSSRESAQ |
| 471 | AQTEAGSAAAQ |
| 472 | AQTEQRHAPAQ |
| 473 | AQTFGKASDAQ |
| 474 | AQTGAASNIAQ |
| 475 | AQTGLSAEGAQ |
| 476 | AQTGTVHSTAQ |
| 477 | AQTHMQVGAAQ |
| 478 | AQTHQPAEKAQ |
| 479 | AQTHTALPDAQ |
| 480 | AQTHTRSMIAQ |
| 481 | AQTINNVVAAQ |
| 482 | AQTKHSTTEAQ |
| 483 | AQTLAGSSIAQ |
| 484 | AQTLRTSDLAQ |
| 485 | AQTLSGLVMAQ |
| 486 | AQTLSTHVVAQ |
| 487 | AQTNITRAQAQ |
| 488 | AQTNSTRNLAQ |
| 489 | AQTNSTRNVAQ |
| 490 | AQTNTHSVTAQ |
| 491 | AQTNTLHTAAQ |
| 492 | AQTNTRTMQAQ |
| 493 | AQTNVSSEIAQ |
| 494 | AQTPTSSSSAQ |
| 495 | AQTQASASGAQ |
| 496 | AQTQGDRGTAQ |
| 497 | AQTQSKSESAQ |
| 498 | AQTQTHVTSAQ |
| 499 | AQTQTVPTNAQ |
| 500 | AQTRTDSNGAQ |
| 501 | AQTSAHANQAQ |
| 502 | AQTSGHSSTAQ |
| 503 | AQTSLKIVPAQ |
| 504 | AQTSLTVNIAQ |
| 505 | AQTSSHSKDAQ |
| 506 | AQTSSNYVSAQ |
| 507 | AQTSSTGVQAQ |
| 508 | AQTSTHIRDAQ |
| 509 | AQTSVAGSNAQ |
| 510 | AQTSVTYQDAQ |
| 511 | AQTTGQITVAQ |
| 512 | AQTTSQALYAQ |
| 513 | AQTTSSSVNAQ |
| 514 | AQTTSTMSTAQ |
| 515 | AQTTTSPAIAQ |
| 516 | AQTVGRAMEAQ |
| 517 | AQTVGVLAAAQ |
| 518 | AQTVRASTDAQ |
| 519 | AQTVVSGTGAQ |
| 520 | AQVANDSFKAQ |
| 521 | AQVARGDSDAQ |
| 522 | AQVASGVATAQ |
| 523 | AQVASRDQFAQ |
| 524 | AQVATRPDAAQ |
| 525 | AQVCHLRTSAQ |
| 526 | AQVDNVTRSAQ |
| 527 | AQVDSRSTPAQ |
| 528 | AQVEPIRICAQ |
| 529 | AQVERLQSGAQ |
| 530 | AQVFGAHSFAQ |
| 531 | AQVGASSSTAQ |
| 532 | AQVGGANLAAQ |
| 533 | AQVGNAPPHAQ |
| 534 | AQVGSNVGQAQ |
| 535 | AQVGSSSVSAQ |
| 536 | AQVGTVSNSAQ |
| 537 | AQVGVNRTNAQ |
| 538 | AQVHLQAHNAQ |
| 539 | AQVHSLPSVAQ |
| 540 | AQVHTKYTDAQ |
| 541 | AQVIAATAEAQ |
| 542 | AQVIWQNQVAQ |
| 543 | AQVKSGSMAAQ |
| 544 | AQVLSGQSPAQ |
| 545 | AQVLSSMSLAQ |
| 546 | AQVLTSSDLAQ |
| 547 | AQVNHGAITAQ |
| 548 | AQVNSHLTGAQ |
| 549 | AQVNTHTSSAQ |
| 550 | AQVNVTRSNAQ |
| 551 | AQVPGGAGFAQ |
| 552 | AQVPSSNVSAQ |
| 553 | AQVQLVANVAQ |
| 554 | AQVQSHGVYAQ |
| 555 | AQVRSGSNIAQ |
| 556 | AQVRTAAQPAQ |
| 557 | AQVSGHEHYAQ |
| 558 | AQVSIHLTQAQ |
| 559 | AQVSNSMATAQ |
| 560 | AQVSNTLLGAQ |
| 561 | AQVSSFQSSAQ |
| 562 | AQVSSLPSPAQ |
| 563 | AQVSTANTSAQ |
| 564 | AQVSTSSLTAQ |
| 565 | AQVSVSSTAAQ |
| 566 | AQVTGSTKLAQ |
| 567 | AQVTSQIMDAQ |
| 568 | AQVTSVSYSAQ |
| 569 | AQVVSGSSHAQ |
| 570 | AQVVTDAYRAQ |
| 571 | AQWINAFKAAQ |
| 572 | AQWSQSNKAAQ |
| 573 | AQWSSRLNSAQ |
| 574 | AQYAHQYNAAQ |
| 575 | AQYDMYYDKAQ |
| 576 | AQYERSKVDAQ |
| 577 | AQYGTMIQVAQ |
| 578 | AQYHSGVGEAQ |
| 579 | AQYKATHIAAQ |
| 580 | AQYKTGNHEAQ |
| 581 | AQYLSNLMMAQ |
| 582 | AQYNGHSNNAQ |
| 583 | AQYNGQSSMAQ |
| 584 | AQYNHTRLLAQ |
| 585 | AQYNSTRASAQ |
| 586 | AQYPSSSSMAQ |
| 587 | AQYQHSSLTAQ |
| 588 | AQYQLVPRSAQ |
| 589 | AQYQTSISPAQ |
| 590 | AQYQTSTQGAQ |
| 591 | AQYSGTHTAAQ |
| 592 | AQYSSGHANAQ |
| 593 | AQYSSGLAGAQ |
| 594 | AQYVNHGLGAQ |

TABLE 2
Brain and Heart Sequences

| SEQ ID | AA sequence |
|---|---|
| NO: | (#587-597) |
| 595 | AQAAGVSAGAQ |
| 596 | AQAGHNMGGAQ |
| 597 | AQANKVKDLAQ |
| 598 | AQANLTRVTAQ |
| 599 | AQANNTMKQAQ |
| 600 | AQANQTRPFAQ |
| 601 | AQANSVKNIAQ |
| 602 | AQANTTKSSAQ |
| 603 | AQANTTRQTAQ |
| 604 | AQANTVRNTAQ |
| 605 | AQARAAPQTAQ |
| 606 | AQASAGSSGAQ |
| 607 | AQASNHTSGAQ |
| 608 | AQASRSEIGAQ |
| 609 | AQASSSAREAQ |
| 610 | AQATGYSGAAQ |
| 611 | AQATHSREQAQ |
| 612 | AQATNSAVGAQ |
| 613 | AQDARPSPNAQ |
| 614 | AQDNHVRTVAQ |
| 615 | AQDNKVKTFAQ |
| 616 | AQDNMIRSIAQ |
| 617 | AQDNSTRISAQ |
| 618 | AQDNSVKHSAQ |
| 619 | AQDNSVRLTAQ |
| 620 | AQDNSVRPSAQ |
| 621 | AQDNSVRSPAQ |
| 622 | AQDNVIRSIAQ |
| 623 | AQDRAQSHSAQ |
| 624 | AQDRSQSHSAQ |
| 625 | AQDTSHRASAQ |
| 626 | AQEGGTGKVAQ |
| 627 | AQENHVRSSAQ |
| 628 | AQENKIKTNAQ |
| 629 | AQENNTRMVAQ |
| 630 | AQENQVRNTAQ |
| 631 | AQENRIINHAQ |
| 632 | AQENSTKSMAQ |
| 633 | AQENSTRVQAQ |
| 634 | AQENTTKHLAQ |
| 635 | AQENTTKSLAQ |
| 636 | AQENTVRNMAQ |
| 637 | AQETAVRSSAQ |
| 638 | AQETHNRAGAQ |
| 639 | AQEVRGGTMAQ |
| 640 | AQFNSTKVVAQ |
| 641 | AQFNSTRSVAQ |
| 642 | AQFNSVRPVAQ |
| 643 | AQGGAPPTNAQ |
| 644 | AQGNAIRNGAQ |
| 645 | AQGNAVKPFAQ |
| 646 | AQGNGTRGFAQ |
| 647 | AQGNGTRPTAQ |
| 648 | AQGNITKVFAQ |
| 649 | AQGNNSKPAAQ |
| 650 | AQGNNTKQMAQ |
| 651 | AQGNNTRLTAQ |
| 652 | AQGNQLKNTAQ |
| 653 | AQGNRIAQVAQ |
| 654 | AQGNRKPNEAQ |
| 655 | AQGNRTQPSAQ |
| 656 | AQGNRTSGGAQ |
| 657 | AQGNRVQNVAQ |
| 658 | AQGNSIINHAQ |
| 659 | AQGNSSKNLAQ |
| 660 | AQGNSVKDSAQ |
| 661 | AQGNSVKGTAQ |
| 662 | AQGNSVRISAQ |
| 663 | AQGNSVRTVAQ |
| 664 | AQGNTIKDYAQ |
| 665 | AQGNTTRHPAQ |
| 666 | AQGNTTRVPAQ |
| 667 | AQGNTVKSEAQ |
| 668 | AQGRNTSMEAQ |
| 669 | AQGRSSQGEAQ |
| 670 | AQGSRGSGAAQ |
| 671 | AQGTRSTDNAQ |
| 672 | AQGTSSNTGAQ |
| 673 | AQGVASSHQAQ |
| 674 | AQHGSSTLGAQ |
| 675 | AQHGSTFTGAQ |
| 676 | AQHIGSIPAAQ |
| 677 | AQHNNVRPVAQ |
| 678 | AQHNSTRNAAQ |
| 679 | AQHNTTKPIAQ |
| 680 | AQHSQSSKDAQ |
| 681 | AQHTSSISSAQ |
| 682 | AQIAHASSMAQ |
| 683 | AQIAHSSSGAQ |
| 684 | AQIISSNVPAQ |
| 685 | AQINATRNQAQ |
| 686 | AQINHVRDTAQ |
| 687 | AQINTTKPIAQ |
| 688 | AQIRSTTMDAQ |
| 689 | AQISAGPREAQ |
| 690 | AQLHGTSTAAQ |
| 691 | AQLNATKPIAQ |
| 692 | AQLNHTKQTAQ |
| 693 | AQLNNTKPIAQ |
| 694 | AQLNNTRPGAQ |
| 695 | AQLNSTARSAQ |
| 696 | AQLNSTKPIAQ |
| 697 | AQLNTAKPIAQ |
| 698 | AQLNTIKPIAQ |
| 699 | AQLNTSKPIAQ |
| 700 | AQLNTTKAIAQ |
| 701 | AQLNTTKHIAQ |
| 702 | AQLNTTKLAAQ |
| 703 | AQLNTTKLIAQ |
| 704 | AQLNTTKPFAQ |
| 705 | AQLNTTKPGAQ |
| 706 | AQLNTTKPIAQ |
| 707 | AQLNTTKPLAQ |
| 708 | AQLNTTKPMAQ |
| 709 | AQLNTTKPNAQ |
| 710 | AQLNTTKPSAQ |
| 711 | AQLNTTKPTAQ |
| 712 | AQLNTTKPVAQ |
| 713 | AQLNTTKSIAQ |
| 714 | AQLNTTKSVAQ |
| 715 | AQLNTTKTIAQ |
| 716 | AQLNTTRPIAQ |
| 717 | AQLNTVKPLAQ |
| 718 | AQLNYVRQVAQ |
| 719 | AQLPQHGSGAQ |
| 720 | AQLPSAHSSAQ |
| 721 | AQLSSPSTAAQ |
| 722 | AQLTNQSSAAQ |
| 723 | AQLVGTSAMAQ |
| 724 | AQLVRNTDLAQ |
| 725 | AQMHNIATPAQ |
| 726 | AQMNHTRREAQ |
| 727 | AQMNNTARPAQ |
| 728 | AQMNNTKQTAQ |
| 729 | AQMNNTRGSAQ |
| 730 | AQMNNTVRAAQ |
| 731 | AQMNSTRAAAQ |
| 732 | AQMPGHGSSAQ |
| 733 | AQMPTQHASAQ |
| 734 | AQMTHSHGSAQ |
| 735 | AQNAHNVSTAQ |
| 736 | AQNNLTRNNAQ |
| 737 | AQNNMTRAIAQ |
| 738 | AQNNMTRNNAQ |
| 739 | AQNNQIKLMAQ |
| 740 | AQNNRSPTLAQ |
| 741 | AQNNSIRLAAQ |
| 742 | AQNNSIRSSAQ |
| 743 | AQNNSTKLLAQ |
| 744 | AQNNSVRPSAQ |
| 745 | AQNNTSKNVAQ |
| 746 | AQNNTTKLAAQ |
| 747 | AQNNTTTRTAQ |
| 748 | AQNNTVKNLAQ |
| 749 | AQNNTVRPSAQ |
| 750 | AQNNVVRSTAQ |
| 751 | AQNNYVRSAAQ |
| 752 | AQNQHSSQTAQ |
| 753 | AQNRGIQSDAQ |
| 754 | AQNSHNVPSAQ |
| 755 | AQNSNNVAGAQ |
| 756 | AQNSRSVSDAQ |
| 757 | AQNSSTRTGAQ |
| 758 | AQNSTKPITAQ |
| 759 | AQNSTRPLTAQ |
| 760 | AQNTRGLVEAQ |
| 761 | AQNTTKGMAAQ |
| 762 | AQNTTRMTTAQ |
| 763 | AQNTTRSESAQ |
| 764 | AQNVRNVLDAQ |
| 765 | AQPAGRNLDAQ |
| 766 | AQPGYGNQGAQ |
| 767 | AQPMSRDTSAQ |
| 768 | AQPNAVKPMAQ |
| 769 | AQPNITRNNAQ |
| 770 | AQPNIVRSTAQ |
| 771 | AQPNQTKTTAQ |
| 772 | AQPNTTKLSAQ |
| 773 | AQPNTTKPIAQ |
| 774 | AQPNTTRSTAQ |
| 775 | AQPNVTRGFAQ |
| 776 | AQPNVTRTVAQ |
| 777 | AQQATVRTEAQ |
| 778 | AQQDRSSAVAQ |
| 779 | AQQHGVSAPAQ |
| 780 | AQQIQRSIDAQ |
| 781 | AQQNAIRQSAQ |
| 782 | AQQNATKGIAQ |
| 783 | AQQNLTRMTAQ |
| 784 | AQQNNVRPSAQ |
| 785 | AQQNSIRSVAQ |
| 786 | AQQNSTRSLAQ |
| 787 | AQQNTTRTSAQ |
| 788 | AQQNYTKVAAQ |
| 789 | AQQRIPSNTAQ |
| 790 | AQQRTSSQEAQ |
| 791 | AQQSSSRLGAQ |
| 792 | AQQVRSDMGAQ |
| 793 | AQRDGLILIAQ |
| 794 | AQRDNIILIAQ |
| 795 | AQRDSTMHAAQ |
| 796 | AQRDTHVFHAQ |
| 797 | AQRGDLHFQAQ |
| 798 | AQRLSQSSDAQ |
| 799 | AQRSGLILFAQ |
| 800 | AQRSGSHFVAQ |
| 801 | AQRVGQSVSAQ |
| 802 | AQRVVGPLFAQ |
| 803 | AQSDRGNHSAQ |
| 804 | AQSDSRNASAQ |
| 805 | AQSESARGNAQ |
| 806 | AQSGHNASGAQ |
| 807 | AQSGSHTNSAQ |
| 808 | AQSGSSRGEAQ |
| 809 | AQSLSQPERAQ |
| 810 | AQSMGHSHAAQ |
| 811 | AQSMSYGSSAQ |
| 812 | AQSNAAKPVAQ |
| 813 | AQSNATTRAAQ |
| 814 | AQSNDIRNNAQ |
| 815 | AQSNDTKNTAQ |
| 816 | AQSNGIRNTAQ |
| 817 | AQSNHTTRSAQ |
| 818 | AQSNLVRQTAQ |
| 819 | AQSNNAKPSAQ |
| 820 | AQSNNSSRPAQ |
| 821 | AQSNQTRGNAQ |
| 822 | AQSNSTKMIAQ |
| 823 | AQSNSTKNAAQ |
| 824 | AQSNSTKNVAQ |
| 825 | AQSNSTKSLAQ |
| 826 | AQSNSTRSSAQ |
| 827 | AQSNSVKAPAQ |
| 828 | AQSNSVKERAQ |
| 829 | AQSNSVKITAQ |
| 830 | AQSNSVKSFAQ |
| 831 | AQSNSVKTVAQ |
| 832 | AQSNSVRLVAQ |
| 833 | AQSNTTKPIAQ |
| 834 | AQSNTTRPMAQ |
| 835 | AQSNTTRQTAQ |
| 836 | AQSNTTTRAAQ |
| 837 | AQSNTVKAMAQ |
| 838 | AQSNTVKNAAQ |
| 839 | AQSNTVRNSAQ |
| 840 | AQSNTVRSSAQ |
| 841 | AQSNVIRNSAQ |
| 842 | AQSNYTRSPAQ |
| 843 | AQSPSRHTEAQ |
| 844 | AQSQHASSAAQ |
| 845 | AQSQSSSGSAQ |
| 846 | AQSSATRVDAQ |
| 847 | AQSSGYAQQAQ |
| 848 | AQSSNSGREAQ |
| 849 | AQSSSGGTGAQ |
| 850 | AQSTAIRQDAQ |
| 851 | AQSTQTRAAAQ |
| 852 | AQSTQVRSEAQ |
| 853 | AQSTRGNDNAQ |
| 854 | AQSTSTHGAAQ |
| 855 | AQTGVRSGDAQ |
| 856 | AQTMRQTNDAQ |
| 857 | AQTNHTREVAQ |
| 858 | AQTNHVKTMAQ |
| 859 | AQTNHVRSAAQ |
| 860 | AQTNLVKPTAQ |
| 861 | AQTNMTRTTAQ |
| 862 | AQTNNATRPAQ |
| 363 | AQTNNAVRAAQ |
| 864 | AQTNNTKSTAQ |
| 865 | AQTNNTLRSAQ |
| 866 | AQTNNTSPGAQ |
| 867 | AQTNNVRAPAQ |
| 868 | AQTNSIKSTAQ |
| 869 | AQTNSIRPIAQ |
| 870 | AQTNSVKLIAQ |
| 871 | AQTNTTKSMAQ |
| 872 | AQTNTVRASAQ |
| 873 | AQTNTVRQTAQ |
| 874 | AQTRGASNEAQ |
| 875 | AQTSGRSDAAQ |
| 876 | AQTVSDRSGAQ |
| 877 | AQVGHSAVSAQ |
| 878 | AQVHNRADVAQ |
| 879 | AQVHSGPVSAQ |
| 880 | AQVNATRNVAQ |
| 881 | AQVNHTKGLAQ |
| 882 | AQVNSLGNNAQ |
| 883 | AQVNSTKSMAQ |
| 884 | AQVNSTKSTAQ |
| 885 | AQVNSTRGNAQ |
| 886 | AQVNTTKPIAQ |
| 887 | AQVNTTQSGAQ |
| 888 | AQVSDTRAGAQ |
| 889 | AQVSHSLASAQ |
| 890 | AQVSSSGMTAQ |
| 891 | AQVSVTRSDAQ |
| 892 | AQWAANTSGAQ |
| 893 | AQYAGVSSPAQ |
| 894 | AQYGSHGVGAQ |
| 895 | AQYNSTKNGAQ |
| 896 | AQYNSTKNMAQ |
| 897 | AQYNSTKSHAQ |
| 898 | AQYNTTKPIAQ |

TABLE 3
Heart Sequences

| SEQ ID | AA sequence |
|---|---|
| NO: | (#587-597) |
| 899 | AQAAHNKDSAQ |
| 900 | AQADARNTSAQ |
| 901 | AQAESRGSAAQ |
| 902 | AQAESRIGSAQ |
| 903 | AQAESRTMPAQ |
| 904 | AQAHAATSAAQ |
| 905 | AQAISLRGDAQ |
| 906 | AQAKGSTLLAQ |
| 907 | AQALARSVSAQ |
| 908 | AQALGQSRVAQ |
| 909 | AQALSHSSGAQ |
| 910 | AQANHTRVAAQ |
| 911 | AQAQGENLLAQ |
| 912 | AQARTGPMLAQ |
| 913 | AQARTHLQDAQ |
| 914 | AQARVGATAAQ |
| 915 | AQARVGTNVAQ |
| 916 | AQASHSRDAAQ |
| 917 | AQASSNKTAAQ |
| 918 | AQASSSTAGAQ |
| 919 | AQATARNAVAQ |
| 920 | AQATGRLPEAQ |
| 921 | AQATRNTDMAQ |
| 922 | AQATSIRGPAQ |
| 923 | AQATVSSRIAQ |
| 924 | AQAVNKPLSAQ |
| 925 | AQAVPQSAGAQ |
| 926 | AQAVPSHHGAQ |
| 927 | AQAVRGAMNAQ |
| 928 | AQAVSGIRIAQ |
| 929 | AQAVSRSVDAQ |
| 930 | AQAVWPTRNAQ |
| 931 | AQAYARGESAQ |
| 932 | AQAYGETFWAQ |
| 933 | AQCLGPCLQAQ |
| 934 | AQDGIRLTPAQ |
| 935 | AQDGRTVSMAQ |
| 936 | AQDGSTRMGAQ |
| 937 | AQDIRSTASAQ |
| 938 | AQDLSTARSAQ |
| 939 | AQDLSTRVAAQ |
| 940 | AQDMSRSTGAQ |
| 941 | AQDNVHSRGAQ |
| 942 | AQDQHNKSQAQ |
| 943 | AQDQKNATGAQ |
| 944 | AQDRAQSNMAQ |
| 945 | AQDRGTSQTAQ |
| 946 | AQDRRSSDLAQ |
| 947 | AQDRSHGHNAQ |
| 948 | AQDRSNTSNAQ |
| 949 | AQDRSTTHAAQ |
| 950 | AQDSLPEPYAQ |
| 951 | AQDSNRAVAAQ |
| 952 | AQDSNRPSNAQ |
| 953 | AQDSRQSKEAQ |
| 954 | AQDSSNRTGAQ |
| 955 | AQDSYTRSAAQ |
| 956 | AQDTHARGLAQ |
| 957 | AQDTRQSFSAQ |
| 958 | AQEASIRSGAQ |
| 959 | AQEGNISRPAQ |
| 960 | AQELTMPPYAQ |
| 961 | AQEMSKANMAQ |
| 962 | AQENQRSGAAQ |
| 963 | AQERGKNLEAQ |
| 964 | AQERGTVSTAQ |
| 965 | AQERNALREAQ |
| 966 | AQESGQRNQAQ |
| 967 | AQESNSYRPAQ |
| 968 | AQESRATTPAQ |
| 969 | AQESTKYHAAQ |
| 970 | AQESTRISGAQ |
| 971 | AQETGRYNPAQ |
| 972 | AQETTSWVFAQ |
| 973 | AQEVNRSSYAQ |
| 974 | AQEVSRGKDAQ |
| 975 | AQEYGKATAAQ |
| 976 | AQFAGMRTAAQ |
| 977 | AQFDSVRNSAQ |
| 978 | AQFGSRDGSAQ |
| 979 | AQFGSRQPEAQ |
| 980 | AQFHNTRSEAQ |
| 981 | AQFNSDRPGAQ |
| 982 | AQFNSTRIVAQ |
| 983 | AQFNVSSTGAQ |
| 984 | AQFQVATRDAQ |
| 985 | AQFSNRTASAQ |
| 986 | AQFSRGVNEAQ |
| 987 | AQGAGALRGAQ |
| 988 | AQGAGRTLEAQ |
| 989 | AQGDRLTHGAQ |
| 990 | AQGFGHSVSAQ |
| 991 | AQGGTAARGAQ |
| 992 | AQGGVRNAEAQ |
| 993 | AQGHHRTVEAQ |
| 994 | AQGHSANLGAQ |
| 995 | AQGIRNQAEAQ |
| 996 | AQGLGTSSNAQ |
| 997 | AQGLNRLTPAQ |
| 998 | AQGLRSSDSAQ |
| 999 | AQGMRTSNDAQ |
| 1000 | AQGNGSRMAAQ |
| 1001 | AQGNRIAREAQ |
| 1002 | AQGRSLTTDAQ |
| 1003 | AQGSKPSQMAQ |
| 1004 | AQGSYANSQAQ |
| 1005 | AQGVAKGNEAQ |
| 1006 | AQGVMGMRNAQ |
| 1007 | AQGVRSGLSAQ |
| 1008 | AQGVRSHDAAQ |
| 1009 | AQGVSNRANAQ |
| 1010 | AQGVSRATPAQ |
| 1011 | AQGVTLGSRAQ |
| 1012 | AQGYNSTPGAQ |
| 1013 | AQGYSGATGAQ |
| 1014 | AQGYSHSSGAQ |
| 1015 | AQHFVDSGLAQ |
| 1016 | AQHGTSRPDAQ |
| 1017 | AQHIVTHDLAQ |
| 1018 | AQHLVDRSLAQ |
| 1019 | AQHMSDSPHAQ |
| 1020 | AQHMVPSLLAQ |
| 1021 | AQHNLPVLHAQ |
| 1022 | AQHNPTRADAQ |
| 1023 | AQHPVQSSLAQ |
| 1024 | AQHQAKLQNAQ |
| 1025 | AQHQIAYDLAQ |
| 1026 | AQHQVPSLLAQ |
| 1027 | AQHRIATVEAQ |
| 1028 | AQHRVSEHFAQ |
| 1029 | AQHSSIGSSAQ |
| 1030 | AQHSVSHHFAQ |
| 1031 | AQHYLSTDFAQ |
| 1032 | AQIASRGLAAQ |
| 1033 | AQIDNRHNGAQ |
| 1034 | AQIGASSGGAQ |
| 1035 | AQIGGAVTTAQ |
| 1036 | AQIGGHERPAQ |
| 1037 | AQIGHVSANAQ |
| 1038 | AQIGRVTNEAQ |
| 1039 | AQIGSRASDAQ |
| 1040 | AQIGSSRLGAQ |
| 1041 | AQIHGKEREAQ |
| 1042 | AQIHLSSATAQ |
| 1043 | AQIHSRGDTAQ |
| 1044 | AQIHSTNPGAQ |
| 1045 | AQIISNRAVAQ |
| 1046 | AQILHLPMNAQ |
| 1047 | AQILTSTVGAQ |
| 1048 | AQINSTATGAQ |
| 1049 | AQIPNFPTLAQ |
| 1050 | AQIPSGNVSAQ |
| 1051 | AQIPSKSPAAQ |
| 1052 | AQIPSTHTGAQ |
| 1053 | AQIPSTRAGAQ |
| 1054 | AQIRHGATDAQ |
| 1055 | AQIRPRPEDAQ |
| 1056 | AQIRSDHSSAQ |
| 1057 | AQIRSDSAMAQ |
| 1058 | AQIRSSTGDAQ |
| 1059 | AQIRSSTIFAQ |
| 1060 | AQIRTMSSEAQ |
| 1061 | AQISNRTSSAQ |
| 1062 | AQISRQEVLAQ |
| 1063 | AQISSHSPGAQ |
| 1064 | AQISSSRLSAQ |
| 1065 | AQISVSLRSAQ |
| 1066 | AQITGVPMNAQ |
| 1067 | AQITRNADNAQ |
| 1068 | AQITSSTQFAQ |
| 1069 | AQITTRIEQAQ |
| 1070 | AQIVKPNITAQ |
| 1071 | AQIVSRTENAQ |
| 1072 | AQIVVRGDSAQ |
| 1073 | AQIYSASTPAQ |
| 1074 | AQKGQPPFLAQ |
| 1075 | AQKHHVPFDAQ |
| 1076 | AQKLDLPMTAQ |
| 1077 | AQKLLGGLSAQ |
| 1078 | AQKLSLPTTAQ |
| 1079 | AQKNGQPVQAQ |
| 1080 | AQKPYVPHDAQ |
| 1081 | AQKQRSEDPAQ |
| 1082 | AQKSGSMLVAQ |
| 1083 | AQKSQSLAGAQ |
| 1084 | AQKSQTSYMAQ |
| 1085 | AQKTTTSLAAQ |
| 1086 | AQLAGRGAGAQ |
| 1087 | AQLAPIMREAQ |
| 1088 | AQLATTRVEAQ |
| 1089 | AQLDRANPQAQ |
| 1090 | AQLEHRSGSAQ |
| 1091 | AQLFGPLLLAQ |
| 1092 | AQLFSWRNAAQ |
| 1093 | AQLFTTSNIAQ |
| 1094 | AQLGHSLTSAQ |
| 1095 | AQLGHSSQIAQ |
| 1096 | AQLGNLGHQAQ |
| 1097 | AQLGRVQSDAQ |
| 1098 | AQLGSSSVSAQ |
| 1099 | AQLHGPTLLAQ |
| 1100 | AQLHGVPMNAQ |
| 1101 | AQLHHNRESAQ |
| 1102 | AQLHNFPDNAQ |
| 1103 | AQLHSIGTSAQ |
| 1104 | AQLHSSTLGAQ |
| 1105 | AQLIHNGAAAQ |
| 1106 | AQLKVGPMDAQ |
| 1107 | AQLKVPVLMAQ |
| 1108 | AQLLDHISKAQ |
| 1109 | AQLLPATRDAQ |
| 1110 | AQLNHGANAAQ |
| 1111 | AQLNHTRPGAQ |
| 1112 | AQLNSPSFLAQ |
| 1113 | AQLNSSRASAQ |
| 1114 | AQLNSVKHYAQ |
| 1115 | AQLNTTKTNAQ |
| 1116 | AQLPGRSTSAQ |
| 1117 | AQLPSYHSAAQ |
| 1118 | AQLQRSTDGAQ |
| 1119 | AQLQTTYSSAQ |
| 1120 | AQLRNSSGDAQ |
| 1121 | AQLRSLSAEAQ |
| 1122 | AQLSHSYSSAQ |
| 1123 | AQLSNSVKTAQ |
| 1124 | AQLSSHAYQAQ |
| 1125 | AQLSSRPDAAQ |
| 1126 | AQLSSSRSAAQ |
| 1127 | AQLSSSYTVAQ |
| 1128 | AQLSTSYSSAQ |
| 1129 | AQLTHVSNIAQ |
| 1130 | AQLTTSVRIAQ |
| 1131 | AQLTTTRTEAQ |
| 1132 | AQLTVAGRSAQ |
| 1133 | AQMGSKSNEAQ |
| 1134 | AQMKVPILLAQ |
| 1135 | AQMLAGRESAQ |
| 1136 | AQMNAGRSGAQ |
| 1137 | AQMNSSKPTAQ |
| 1138 | AQMNSVRTGAQ |
| 1139 | AQMNTSRSPAQ |
| 1140 | AQMNTTRVPAQ |
| 1141 | AQMPTKMTDAQ |
| 1142 | AQMRTGSNEAQ |
| 1143 | AQMRTLTGDAQ |
| 1144 | AQMRTSHTEAQ |
| 1145 | AQMRTSNLTAQ |
| 1146 | AQMSGHRQDAQ |
| 1147 | AQMSHSSGAAQ |
| 1148 | AQMSNRVLEAQ |
| 1149 | AQMSTTRSSAQ |
| 1150 | AQMTSKPESAQ |
| 1151 | AQMVNRVQSAQ |
| 1152 | AQMVRSNDPAQ |
| 1153 | AQMYTTGQSAQ |
| 1154 | AQNAGRYGDAQ |
| 1155 | AQNGARTPPAQ |
| 1156 | AQNGQRLSPAQ |
| 1157 | AQNGRVGSNAQ |
| 1158 | AQNGSATRVAQ |
| 1159 | AQNGSKLAGAQ |
| 1160 | AQNGSRVIAAQ |
| 1161 | AQNGTMRLAAQ |
| 1162 | AQNHRPSGDAQ |
| 1163 | AQNLANTRDAQ |
| 1164 | AQNLQPGKIAQ |
| 1165 | AQNLRGTSVAQ |
| 1166 | AQNLSSVNGAQ |
| 1167 | AQNRTGGFDAQ |
| 1168 | AQNRVAQGEAQ |
| 1169 | AQNSATMRQAQ |
| 1170 | AQNSDPYRFAQ |
| 1171 | AQNSHISRDAQ |
| 1172 | AQNSQTLRSAQ |
| 1173 | AQNSRAAEHAQ |
| 1174 | AQNSRGPVAAQ |
| 1175 | AQNSSHANSAQ |
| 1176 | AQNSTQYNAAQ |
| 1177 | AQNTGHASMAQ |
| 1178 | AQNTGRSDSAQ |
| 1179 | AQNTNMPQYAQ |
| 1180 | AQNTSSVRGAQ |
| 1181 | AQNTVNVSGAQ |
| 1182 | AQNVAGRLDAQ |
| 1183 | AQNVSNLHGAQ |
| 1184 | AQNVSQLRDAQ |
| 1185 | AQNVTRGHEAQ |
| 1186 | AQNVYNVSGAQ |
| 1187 | AQPARPNNPAQ |
| 1188 | AQPFTPKPMAQ |
| 1189 | AQPGVGKQPAQ |
| 1190 | AQPHDSVMWAQ |
| 1191 | AQPINLPMSAQ |
| 1192 | AQPKSSHEWAQ |
| 1193 | AQPNNTIRGAQ |
| 1194 | AQPQKTGSAAQ |
| 1195 | AQPQSGPLPAQ |
| 1196 | AQPQSNRPAAQ |
| 1197 | AQPQSRGDSAQ |
| 1198 | AQPRLPEVFAQ |
| 1199 | AQPRTGTPPAQ |
| 1200 | AQPSHSVLFAQ |
| 1201 | AQPSRGQTAAQ |
| 1202 | AQPSRGQTTAQ |
| 1203 | AQPSTDTFWAQ |
| 1204 | AQPSVTGRSAQ |
| 1205 | AQPSYGGSGAQ |
| 1206 | AQPTGLPINAQ |
| 1207 | AQPTQRTGTAQ |
| 1208 | AQPTRGTGTAQ |
| 1209 | AQPTSALPMAQ |
| 1210 | AQPVSRSPAAQ |
| 1211 | AQQAGRSVAAQ |
| 1212 | AQQAGSSVAAQ |
| 1213 | AQQASRNEIAQ |
| 1214 | AQQATRGDSAQ |
| 1215 | AQQDRILSAAQ |
| 1216 | AQQDSMRGLAQ |
| 1217 | AQQDTRSHAAQ |
| 1218 | AQQGGLILLAQ |
| 1219 | AQQGSIRSDAQ |
| 1220 | AQQGYGGQTAQ |
| 1221 | AQQHAPSLLAQ |
| 1222 | AQQLPGGLHAQ |
| 1223 | AQQLSNKTNAQ |
| 1224 | AQQLSRDVPAQ |
| 1225 | AQQLVQHRDAQ |
| 1226 | AQQMRTGGIAQ |
| 1227 | AQQPGYSSDAQ |
| 1228 | AQQPRANQIAQ |
| 1229 | AQQPRTNESAQ |
| 1230 | AQQQLPVLMAQ |
| 1231 | AQQRESPLDAQ |
| 1232 | AQQRHLPTDAQ |
| 1233 | AQQSAGKLGAQ |
| 1234 | AQQSLSSLSAQ |
| 1235 | AQQSNLPMTAQ |
| 1236 | AQQSNVYREAQ |
| 1237 | AQQTSRLQEAQ |
| 1238 | AQQTTDRSGAQ |
| 1239 | AQQVGPILMAQ |
| 1240 | AQQVGSYQHAQ |
| 1241 | AQQVSTARPAQ |
| 1242 | AQQYGSVSQAQ |
| 1243 | AQQYSSGSSAQ |
| 1244 | AQRAGVSQIAQ |
| 1245 | AQRDTTYTTAQ |
| 1246 | AQRERSSEVAQ |
| 1247 | AQRGHGSSDAQ |
| 1248 | AQRGPLTLLAQ |
| 1249 | AQRGTLDLLAQ |
| 1250 | AQRIASPYDAQ |
| 1251 | AQRIGTATVAQ |
| 1252 | AQRIGTMSAAQ |
| 1253 | AQRLPGGLSAQ |
| 1254 | AQRLTQHEPAQ |
| 1255 | AQRMSNGSTAQ |
| 1256 | AQRMSSSSDAQ |
| 1257 | AQRNHSAGDAQ |
| 1258 | AQRNLPQVYAQ |
| 1259 | AQRNYSPTEAQ |
| 1260 | AQRPEVPNWAQ |
| 1261 | AQRPQGSAGAQ |
| 1262 | AQRPTMPEYAQ |
| 1263 | AQRSDRLTDAQ |
| 1264 | AQRSGMAVEAQ |
| 1265 | AQRSGSGSDAQ |
| 1266 | AQRSGSSADAQ |
| 1267 | AQRSNLLAEAQ |
| 1268 | AQRSNSSVGAQ |
| 1269 | AQRSTAIVAAQ |
| 1270 | AQRTGMTGAAQ |
| 1271 | AQRTGSVVNAQ |
| 1272 | AQRTGTQSSAQ |
| 1273 | AQRTIGTSTAQ |
| 1274 | AQRTSQIQSAQ |
| 1275 | AQRVGASGTAQ |
| 1276 | AQRVGSSLSAQ |
| 1277 | AQRVPAGGGAQ |
| 1278 | AQRVSATQTAQ |
| 1279 | AQRVVGGLTAQ |
| 1280 | AQSALHVSGAQ |
| 1281 | AQSASLGGYAQ |
| 1282 | AQSASLRSDAQ |
| 1283 | AQSAVVRSGAQ |
| 1284 | AQSDMPRTFAQ |
| 1285 | AQSDTRSMSAQ |
| 1286 | AQSFNHGATAQ |
| 1287 | AQSFTRIPPAQ |
| 1288 | AQSGNFSGGAQ |
| 1289 | AQSGSALHGAQ |
| 1290 | AQSGSRLLDAQ |
| 1291 | AQSGSSRDMAQ |
| 1292 | AQSGSSRPSAQ |
| 1293 | AQSGSYSTVAQ |
| 1294 | AQSHEKFLYAQ |
| 1295 | AQSHSASREAQ |
| 1296 | AQSHSNTGNAQ |
| 1297 | AQSIPGKGMAQ |
| 1298 | AQSIRSQLPAQ |
| 1299 | AQSISAGRVAQ |
| 1300 | AQSISGRSDAQ |
| 1301 | AQSISGRVGAQ |
| 1302 | AQSISRQSEAQ |
| 1303 | AQSISRSGYAQ |
| 1304 | AQSLAAEFWAQ |
| 1305 | AQSLDPLGLAQ |
| 1306 | AQSLIHSIWAQ |
| 1307 | AQSLNRVVEAQ |
| 1308 | AQSLQRSVDAQ |
| 1309 | AQSLSRDGTAQ |
| 1310 | AQSLVKSGQAQ |
| 1311 | AQSMSRLNEAQ |
| 1312 | AQSNTLRNVAQ |
| 1313 | AQSNYVRSPAQ |
| 1314 | AQSPRSNVTAQ |
| 1315 | AQSPRSSIGAQ |
| 1316 | AQSPVAPNWAQ |
| 1317 | AQSQALRADAQ |
| 1318 | AQSQGVRVPAQ |
| 1319 | AQSQKMLHGAQ |
| 1320 | AQSQPGPFNAQ |
| 1321 | AQSQSAKGPAQ |
| 1322 | AQSRGQTTDAQ |
| 1323 | AQSRGSETTAQ |
| 1324 | AQSRISNVSAQ |
| 1325 | AQSRQTGLTAQ |
| 1326 | AQSRSQATDAQ |
| 1327 | AQSRSTDTGAQ |
| 1328 | AQSRVGLSTAQ |
| 1329 | AQSSAGKSPAQ |
| 1330 | AQSSAGRTGAQ |
| 1331 | AQSSATVREAQ |
| 1332 | AQSSDSRSSAQ |
| 1333 | AQSSGISRDAQ |
| 1334 | AQSSGPIILAQ |
| 1335 | AQSSHSANMAQ |
| 1336 | AQSSIVRQEAQ |
| 1337 | AQSSQNVSGAQ |
| 1338 | AQSSQRGHEAQ |
| 1339 | AQSSSSRGPAQ |
| 1340 | AQSSTVRNAAQ |
| 1341 | AQSSVTHSSAQ |
| 1342 | AQSSYQSNGAQ |
| 1343 | AQSTAARPVAQ |
| 1344 | AQSTGHNTQAQ |
| 1345 | AQSTGSSTSAQ |
| 1346 | AQSTGTPNWAQ |
| 1347 | AQSTHIARDAQ |
| 1348 | AQSTIVYREAQ |
| 1349 | AQSTMQNRIAQ |
| 1350 | AQSTNHGNNAQ |
| 1351 | AQSTQIHAGAQ |
| 1352 | AQSTQLRSGAQ |
| 1353 | AQSTRALQPAQ |
| 1354 | AQSTRSSDSAQ |
| 1355 | AQSTRTAPEAQ |
| 1356 | AQSTSNRPAAQ |
| 1357 | AQSTSPPRPAQ |
| 1358 | AQSTSRLPAAQ |
| 1359 | AQSTSSHTPAQ |
| 1360 | AQSTSSRTSAQ |
| 1361 | AQSTSSRTTAQ |
| 1362 | AQSTTSAPGAQ |
| 1363 | AQSTVTAVGAQ |
| 1364 | AQSVGELPFAQ |
| 1365 | AQSVPAKGSAQ |
| 1366 | AQSVRAGLNAQ |
| 1367 | AQSVRSPVTAQ |
| 1368 | AQSVRSSDYAQ |
| 1369 | AQSVTRTVAAQ |
| 1370 | AQSVYSHSSAQ |
| 1371 | AQSYAPQLLAQ |
| 1372 | AQSYSNRSVAQ |
| 1373 | AQTAAVRSGAQ |
| 1374 | AQTALKAVGAQ |
| 1375 | AQTANTRAEAQ |
| 1376 | AQTASQLSAAQ |
| 1377 | AQTDRITISAQ |
| 1378 | AQTDTRHATAQ |
| 1379 | AQTERNTASAQ |
| 1380 | AQTFHAVKEAQ |
| 1381 | AQTGSLAHGAQ |
| 1382 | AQTIGMRSAAQ |
| 1383 | AQTINKPNAAQ |
| 1384 | AQTISNSSGAQ |
| 1385 | AQTITRGTDAQ |
| 1386 | AQTLQNVNYAQ |
| 1387 | AQTLSSSREAQ |
| 1388 | AQTMGRNVSAQ |
| 1389 | AQTQGNRTDAQ |
| 1390 | AQTQHSTQSAQ |
| 1391 | AQTQSAYGSAQ |
| 1392 | AQTQSLRHDAQ |
| 1393 | AQTQTDRHSAQ |
| 1394 | AQTQTNIGRAQ |
| 1395 | AQTRHSSSDAQ |
| 1396 | AQTRSSSEAAQ |
| 1397 | AQTRSTIQEAQ |
| 1398 | AQTSATYSGAQ |
| 1399 | AQTSGTRHEAQ |
| 1400 | AQTSHAPNNAQ |
| 1401 | AQTSHSSSVAQ |
| 1402 | AQTSHVSSAAQ |
| 1403 | AQTSILRNEAQ |
| 1404 | AQTSNSSGQAQ |
| 1405 | AQTSNYGATAQ |
| 1406 | AQTSQSRVEAQ |
| 1407 | AQTSRANDTAQ |
| 1408 | AQTSRSNETAQ |
| 1409 | AQTSTPNKIAQ |
| 1410 | AQTSYSTTTAQ |
| 1411 | AQTTARNLEAQ |
| 1412 | AQTTGILRDAQ |
| 1413 | AQTTGTPRPAQ |
| 1414 | AQTTGYSQAAQ |
| 1415 | AQTTIRNAVAQ |
| 1416 | AQTTNITRTAQ |
| 1417 | AQTTSMGKVAQ |
| 1418 | AQTTSMSGHAQ |
| 1419 | AQTTSPKLPAQ |
| 1420 | AQTTSSSYNAQ |
| 1421 | AQTTSTHNAAQ |
| 1422 | AQTTSVRSTAQ |
| 1423 | AQTTTAGKVAQ |
| 1424 | AQTTTKFTPAQ |
| 1425 | AQTVITASGAQ |
| 1426 | AQTVQSHSTAQ |
| 1427 | AQTVSSGRPAQ |
| 1428 | AQTYNATRSAQ |
| 1429 | AQVAGRNVSAQ |
| 1430 | AQVAGTSQGAQ |
| 1431 | AQVAGYSRDAQ |
| 1432 | AQVATPSFLAQ |
| 1433 | AQVFTNRSAAQ |
| 1434 | AQVGHGSLPAQ |
| 1435 | AQVGSYSRDAQ |
| 1436 | AQVGTLSSSAQ |
| 1437 | AQVGVNVHGAQ |
| 1438 | AQVIRGLEQAQ |
| 1439 | AQVISRDSSAQ |
| 1440 | AQVISSHHSAQ |
| 1441 | AQVKANQATAQ |
| 1442 | AQVLHASMSAQ |
| 1443 | AQVLHGSSYAQ |
| 1444 | AQVLRSNVMAQ |
| 1445 | AQVNLPVLLAQ |
| 1446 | AQVPGHSGLAQ |
| 1447 | AQVPGQSSLAQ |
| 1448 | AQVPRSSTDAQ |
| 1449 | AQVPRTGLTAQ |
| 1450 | AQVPTSHSTAQ |
| 1451 | AQVQSHGVNAQ |
| 1452 | AQVQYGGLTAQ |
| 1453 | AQVRGLYSDAQ |
| 1454 | AQVRHSGMEAQ |
| 1455 | AQVRPPNPSAQ |
| 1456 | AQVRSTLTDAQ |
| 1457 | AQVRSVGLDAQ |
| 1458 | AQVRTPLFAAQ |
| 1459 | AQVRTSYSDAQ |
| 1460 | AQVSGIRGDAQ |
| 1461 | AQVSGRSLSAQ |
| 1462 | AQVSNSYLGAQ |
| 1463 | AQVSNTYSGAQ |
| 1464 | AQVSRSESAAQ |
| 1465 | AQVSSYPNSAQ |
| 1466 | AQVTGLRGIAQ |
| 1467 | AQVTHASLSAQ |
| 1468 | AQVTQLGRSAQ |
| 1469 | AQVTRSAGEAQ |
| 1470 | AQVTSKNDLAQ |
| 1471 | AQVTTKTQLAQ |
| 1472 | AQVVANTSRAQ |
| 1473 | AQVVGSNREAQ |
| 1474 | AQVVRSGNEAQ |
| 1475 | AQVVRSVNDAQ |
| 1476 | AQVVRTPNSAQ |
| 1477 | AQWGSHPTGAQ |
| 1478 | AQWLKSVSSAQ |
| 1479 | AQWRHTSAEAQ |
| 1480 | AQYAGNSGNAQ |
| 1481 | AQYASSSNNAQ |
| 1482 | AQYERNHNAAQ |
| 1483 | AQYKSSLSEAQ |
| 1484 | AQYLHGLQQAQ |
| 1485 | AQYNQIRGDAQ |
| 1486 | AQYNSSNFGAQ |
| 1487 | AQYPGNRMEAQ |
| 1488 | AQYRNTVQDAQ |
| 1489 | AQYRQSSSDAQ |
| 1490 | AQYRSGTQEAQ |
| 1491 | AQYTGSISVAQ |
| 1492 | AQYTSSVTGAQ |
| 1493 | AQYVASSPMAQ |
| 1494 | AQYVGSGTAAQ |
| 1495 | AQYVHSNNAAQ |
| 1496 | AQYYSSSGGAQ |

### DETAILED DESCRIPTION OF THE DISCLOSURE

In certain aspects the disclosure provides modified rAAVs with increased expression levels in the brain, heart, or brain and heart when compared to a parent AAV (e.g., AAV9).

In certain aspects, the disclosure provides rAAVs with a peptide insertion and or substitution comprising or consisting of an amino-acid sequence set forth in any one of Tables 1-3.

In certain embodiments, the parental AAV is AAV9. In some embodiments, the parental AAV comprises SEQ ID NO: 1. In various embodiments, the AAV capsid protein comprises a 7-mer insertion inserted into the parental AAV between amino acid 588 and amino acid 589 of the parent AAV, wherein positions 587-597 of the mutant AAV capsid protein (with AA position numbering including the insertion) are selected from the sequence provided in Tables 1-3 or selected from the group consisting of SEQ ID NO: 2-1496.

Generally, the insertion comprises a seven-amino acid sequence (7-mer) that is inserted or substituted at the 588 loop in a parental AAV capsid protein. Aspects provided herein provide amino acid insertions comprising seven amino acid polymer (7-mer) inserted at AA588-589 to produce an eleven amino acid polymer (11-mer) at the 588 loop of a parental AAV capsid protein.

Also disclosed herein are methods and kits for producing therapeutic recombinant AAV (rAAV) particles, as well as methods and pharmaceutical compositions or formulations comprising the rAAV particles, for the treatment of a disease or condition affecting the brain, heart, or brain and heart.

Disclosed herein are AAV capsids engineered with increased viral transduction in the brain, heart, or brain and heart. Disclosed further herein AAV capsids engineered with increased viral transduction in the heart. Advantageously, disclosed herein are AAV capsids engineered with increased viral transduction in the brain, heart, or brain and heart.

The AAV capsids can encapsidate a viral vector with a heterologous nucleic acid encoding, for example, a therapeutic gene expression product. Transduction of the heterologous nucleic acid in the brain, heart, or brain and heart can be achieved upon systemic delivery to a subject of the AAV capsid of the present disclosure encapsidating a heterologous nucleic acid. The AAV capsids disclosed herein are advantageous for many applications of gene therapy to treat human disease, including, but not limited to, disorders of the brain and/or heart.

The recombinant AAV vectors comprising a nucleic acid sequence encoding the AAV capsid proteins of the present disclosure as also provided herein. For example, the viral vectors of the present disclosure comprise a nucleic acid sequence comprising the AAV viral Cap (Capsid) encoding VP1, VP2, and VP3, at least one of which is modified to produce the AAV capsid proteins of the present disclosure. The recombinant AAV vector provided can be derived from an AAV serotype (e.g., AAV9) or a variant AAV serotype including an insertion of the present invention.

### AAV Capsids

Provided herein are modified adeno-associated (AAV) virus capsid compositions useful for integrating a transgene into a target cell or environment (in a subject when they are administered systemically to the subject.

An rAAV comprises an AAV capsid that can be engineered to encapsidate a heterologous nucleic acid (e.g., therapeutic nucleic acid, gene editing machinery). The AAV capsid is made up of three AAV capsid protein monomers, VP1, VP2, and VP3. Sixty copies of these three VP proteins interact in a 1:1:10 ratio to form the viral capsid. VP1 covers the whole of VP2 protein in addition to a ˜137 amino acid N-terminal region (VP1u), VP2 covers the whole of VP3 in addition to ˜65 amino acid N-terminal region (VP1/2 common region). The three capsid proteins share a conserved amino acid sequence of VP3, which in some cases is the region beginning at amino acid position 138 (e.g., AA139-736).

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

The AAV VP3 structure contains highly conserved regions that are common to all serotypes, a core eight-stranded β-barrel motif (βB-βI) and a small α-helix (αA). The loop regions inserted between the β-strands consist of the distinctive HI loop between β-strands H and I, the DE loop between β-strands D and E, and nine variable regions (VRs), which form the top of the loops. These VRs, such as the AA588 loop, are found on the capsid surface and can be associated with specific functional roles in the AAV life cycle including receptor binding, transduction and antigenic specificity.

In some aspects, the rAAV variant of the present invention comprises an AAV capsid protein having a peptide insertion at the residues corresponding to amino acids 588-589 of the AAV9 native sequence of SEQ ID NO: 1.

The AAV capsids comprise AAV capsid proteins (e.g., VP1, VP2, and VP3), each with an insertion, such as in the 588 loop of a parental AAV capsid protein structure (AAV9 VP1 numbering). The 588 loop contains the site of heparan sulfate binding of AAV2 and is amenable to peptide display. The only known receptors for AAV9 is N-linked terminal galactose and AAV receptor (AAVR), but many indications point toward there being others. Modifications to AAV9 588 loop are shown herein to confer an increased transgene transduction in target in vivo environments.

The present invention provides, in an aspect, a peptide insertion at the AAV 588 loop comprising or consisting of an amino-acid sequence set forth in any one of Tables 1-3.

Disclosed herein are AAV capsids comprising AAV capsid proteins with an insertion at the 588 loop that confer a higher transduction in brain cell types (e.g., brain endothelial cells, neurons, astrocytes) and/or heart cell types (e.g., cardiomyocytes, vascular cells, stromal cells, mesothelial cells). In particular, the AAV capsid proteins disclosed herein enable rAAV-mediated transduction of a heterologous nucleic acid (e.g., transgene) in the brain, heart, or brain and heart of a subject. The AAV capsids of the present disclosure may be formulated as a pharmaceutical composition. In addition, the AAV capsids can be isolated and purified to be used for a variety of applications.

In some embodiments, the rAAV capsid of the present disclosure are generated using the methods disclosed herein. In some instances, the rAAV capsid is chimeric. In some instances, the rAAV, or variant AAV protein comprises therein, confer an increase in a localization of the rAAV within the target tissue, as compared to the parental AAV capsid or capsid protein.

### AAV Capsid Proteins

Disclosed herein are recombinant AAV (rAAV) capsids which comprise AAV capsid proteins that are engineered with a modified capsid protein (e.g., VP1, VP2, VP3). In some embodiments, the rAAV capsid proteins of the present disclosure are generated using the methods disclosed herein. In some embodiments, the AAV capsid proteins are used in the methods of delivering a therapeutic nucleic acid (e.g., a transgene) to a subject. In some instances, the rAAV capsid proteins have desired AAV expression rendering them particularly suitable for certain therapeutic applications, e.g., the treatment of a disease or disorder in a subject such as those disclosed herein.

The rAAV capsid proteins may be engineered for optimized expression in the CNS, for example the brain, of a subject upon systemic administration of the rAAV to the subject. The rAAV capsid proteins are engineered to include the insertions provided in Tables 1-2. The rAAV capsid proteins including the insertions provided in Tables 1-2 are engineered to achieve efficient transduction of an encapsidated transgene. In particular, the rAAV capsid proteins have increased expression in the brain of a subject.

The rAAV capsid proteins may also be engineered for optimized expression in the heart of a subject upon systemic administration of the rAAV to the subject. The rAAV capsid proteins are engineered to include the insertions provided in Tables 2-3. The rAAV capsid proteins including the insertions provided in Tables 2-3 are engineered to achieve efficient transduction of an encapsidated transgene. In particular, the rAAV capsid proteins have increased expression in the heart of a subject.

The rAAV capsid proteins may also be engineered for optimized expression in the brain and heart of a subject upon systemic administration of the rAAV to the subject. The rAAV capsid proteins are engineered to include the insertions provided in Table 2. The rAAV capsid proteins including the insertions provided in Table 2 are engineered to achieve efficient transduction of an encapsidated transgene. In particular, the rAAV capsid proteins have increased expression in the brain and heart of a subject.

The engineered AAV capsid proteins described herein have, in some cases, an insertion of an amino acid that is heterologous to the parental AAV capsid protein at amino acid positions in the 588 loop. In some embodiments, the amino acid is not endogenous to the parental AAV capsid protein at the amino acid position of the insertion. The amino acid may be a naturally occurring amino acid in the same or equivalent amino acid position as the insertion of the substitution in a different AAV capsid protein.

The 7-mers described herein were advantageously generated using polymerase chain reaction (PCR) with degenerate primers, where each of the seven amino acids is encoded by a deoxyribose nucleic acid (DNA) sequence. This method of generating random 7-mer amino acid sequences enables sampling of the 207 (1.28 billion) possible 7mer amino acid combinations at the protein level.

The rAAV capsid proteins of the present disclosure comprise an insertion of an amino acid in an amino acid sequence of an AAV capsid protein. The AAV capsid, from which an engineered AAV capsid protein of the present disclosure is produced, is referred to as a “parental” AAV capsid. The complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562.

In some cases, the parental AAV is derived from an AAV with a serotype selected from AAV1, AAV2, AAV3, AAV4, AAV5, AAV6, AAV7, AAV8, AAV9, AAV10, AAV11 and AAV12. The AAV capsid protein that is “derived” from another may be a variant AAV capsid protein. A variant may include, for example, a heterologous amino acid in an amino acid sequence of the AAV capsid protein. The heterologous amino acid may be non-naturally occurring in the AAV capsid protein. The heterologous amino acid may be naturally occurring in a different AAV capsid protein. In some instances, the parental AAV capsid is described in US Pat Publication 2020/0165576 and U.S. Pat. App. Ser. No. 62/832,826 and PCT/US20/20778; the content of each of which is incorporated herein.

In some instances, the parental AAV is AAV9. In some instances, the amino acid sequence of the AAV9 capsid protein comprises SEQ ID NO: 1. The amino acid sequence of AAV9 VP1 capsid protein is provided in SEQ ID NO: 1

(MAADGYLPDWLEDNLSEGIREWWALKPGAPQPKANQQHQDNARGLVLP
GYKYLGPGNGLDKGEPVNAADAAALEHDKAYDQQLKAGDNPYLKYNHAD
AEFQERLKEDTSFGGNLGRAVFQAKKRLLEPLGLVEEAAKTAPGKKRPV
EQSPQEPDSSAGIGKSGAQPAKKRLNFGQTGDTESVPDPQPIGEPPAAP
SGVGSLTMASGGGAPVADNNEGADGVGSSSGNWHCDSQWLGDRVITTST
RTWALPTYNNHLYKQISNSTSGGSSNDNAYFGYSTPWGYFDFNRFHCHF
SPRDWQRLINNNWGFRPKRLNFKLFNIQVKEVTDNNGVKTIANNLTSTV
QVFTDSDYQLPYVLGSAHEGCLPPFPADVFMIPQYGYLTLNDGSQAVGR
SSFYCLEYFPSQMLRTGNNFQFSYEFENVPFHSSYAHSQSLDRLMNPLI
DQYLYYLSKTINGSGQNQQTLKFSVAGPSNMAVQGRNYIPGPSYRQQRV
STTVTQNNNSEFAWPGASSWALNGRNSLMNPGPAMASHKEGEDRFFPLS
GSLIFGKQGTGRDNVDADKVMITNEEEIKTTNPVATESYGQVATNHQSA
QAQAQTGWVQNQGILPGMVWQDRDVYLQGPIWAKIPHTDGNFHPSPLMG
GFGMKHPPPQILIKNTPVPADPPTAFNKDKLNSFITQYSTGQVSVEIEW
ELQKENSKRWNPEIQYTSNYYKSNNVEFAVNTEGVYSEPRPIGTRYLTR
NL).

In some instances, the parental AAV capsid protein sequence is 70%, 75%, 80%, 85%, 90%, 91%, 92%, 93%, 94%, 95%, 96%, 97%, 98%, 99%, or 100% homologous to SEQ ID NO: 1.

AAV capsid proteins from native AAV serotypes, such as AAV9, with tropisms including the liver activate the innate immune response, which in some cases causes a severe inflammatory response in a subject, which can lead to multi-organ failure. By improving transduction of a native AAV serotype for a target in vivo tissue (e.g., brain and/or heart) the rAAV particles of the present disclosure reduce the immunogenic properties of AAV-mediated transgene delivery and prevent activation of the innate immune response. Moreover, with increased transduction efficiency, the amount of the AAV composition needed to be delivered for effective transgene expression can be reduced. By reducing the total volume of the transgene delivered, off-target delivery and expression of the transgene is reduced.

In some instances, the parental AAV capsid protein comprises the entire VP1 region provided in SEQ ID NO: 1 (e.g., amino acids 1-736). In some instances, the parental AAV capsid protein comprises amino acids 217-736 in SEQ ID NO: 1, which is the common region found in VP1, VP2 and VP3 AAV9 capsid proteins. In some instances, the AAV capsid protein comprises amino acids 64-736 in SEQ ID NO: 1, which is the common region found in VP1 and VP2. The parental AAV capsid protein sequence may comprise amino acids selected from 1-736, 10-736, 20-736, 30-736, 40-736, 50-736, 60-736, 70-736, 80-736, 90-736, 100-736, 110-736, 120-736, 130-736, 140-736, 150-736, 160-736, 170-736, 180-736, 190-736, 200-736, 210-736, 220-736, 230-736, 240-736, 250-736, 260-736, 270-736, 280-736, 290-736, 300-736, 310-736, 320-736, 330-736, 340-736, 350-736, 360-736, 370-736, 380-736, 390-736, 400-736, 410-736, 420-736, 430-736, 440-736, and 450-736, from SEQ ID NO: 1. In some aspects, the rAAV variant comprises an AAV capsid protein comprising an amino acid sequence that is at least 98% identical to amino acid 217 to amino acid 736 of SEQ ID NO: 1. In some instances, the amino acid insertion is at a three (3)-fold axis of symmetry of a corresponding parental AAV capsid protein.

Disclosed herein are insertions of an amino acid sequence in an AAV capsid protein. Where the sequence numbering designation “588-589” is noted for AAV9, for example AAV VP1, the invention also includes insertions in similar locations in the other AAV serotypes. As used herein, “AA588-589” indicates that the insertion of the amino acid (or amino acid sequence) is immediately after an amino acid (AA) at position 588 and immediately before an AA at position 589 within an amino acid sequence of a parental AAV VP capsid protein (VP1 numbering). Amino acids 587-591 include a motif comprising “AQAQA” as set forth in SEQ ID NO: 1. It is envisioned that the sequences disclosed herein (Tables 1-3) may be inserted at AA588-589 in an amino acid sequence of a parental AAV9 capsid protein, a variant thereof, or equivalent amino acid position of a parental AAV of a different serotype (e.g., AAV1, AAV2, AAV3, and the like). In certain embodiments, the aforementioned “AQAQ” sequence flanking the insertion may include one or more substitutions.

The insertions described herein may, in some cases, comprise a 7-mer insertion at AA588-589.

Disclosed herein are AAV capsid proteins with an insertion described above in a parental AAV capsid protein that confers an increased transduction in the brain, heart, or brain and heart in a subject, even when delivered systemically. One of the many advantages of the AAV capsid proteins described herein is their ability to target tissue and cells within the brain, heart, or brain and heart. The tissue can be the brain, heart, or brain and heart.

Non-limiting examples of brain cells include a neuron and a glial cell. Glial cells can be selected from an oligodendrocyte, an ependymal cell, an astrocyte and a microglia.

Non-limiting examples of heart cells include cardiomyocytes, vascular cells, stromal cells, mesothelial cells

In some instances, the AAV capsid protein comprises an insertion/substitution of at least or about seven, eight, nine, ten or eleven amino acids of an amino acid sequence of Tables 1-3 at an amino acid position 588-589 in a parental AAV9 capsid protein (SEQ ID NO: 1).

In some cases, the AAV capsid protein has an increased viral transduction enrichment in brain, heart, or brain and heart.

The rAAV capsid proteins described herein may be isolated and purified. The AAV may be isolated and purified by methods standard in the art such as by column chromatography, iodixanol gradients, or cesium chloride gradients. Methods for purifying AAV from helper virus are known in the art and may include methods disclosed in, for example, Clark et al., Hum. Gene Ther., 10 (6): 1031-1039 (1999); Schenpp and Clark, Methods Mol. Med., 69:427-443 (2002); U.S. Pat. No. 6,566,118 and WO 98/09657.

In addition, the AAV capsid proteins disclosed herein, either isolated and purified, or not, may be formulated into a pharmaceutical formulation, which in some cases, further comprises a pharmaceutically acceptable carrier.

The rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein.

“Percent Identity” is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see: Henikoff and Henikoff, (1989) Proc. Natl. Acad. Sci. USA 89:10915).

Sequence identity/similarity values provided herein can refer to the value obtained using the BLAST+ 2.5.0 suite of programs using default settings (blast.ncbi.nlm.nih.gov) (Camacho, C., et al. (2009) BLAST+: architecture and applications. BMC Bioinformatics 10:421).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Ci-ayerie and States (1993) Comput. Chem. 17:191-201) low-complexity filters can be employed alone or in combination.

The terms “substantial identity” and “substantially identical” indicate that a polypeptide or nucleic acid comprises a sequence with between 55-100% sequence identity to a reference sequence, with at least 55% sequence identity, or at least 60%, or at least 65%, or at least 70%, or at least 75%, or at least 80%, or at least 85%, or at least 90%, or at least 95%, or at least 99% sequence identity or any percentage of value within the range of 55-100% sequence identity relative to the reference sequence. The percent sequence identity may occur over a specified comparison window. Optimal alignment may be ascertained or conducted using the homology alignment algorithm of Needleman and Wunsch, supra.

For example, the insertion sequences may include, but are not limited to, sequences that are not exactly the same as the sequences disclosed herein, but which have, in addition to the substitutions explicitly described for various sequences listed herein, additional substitutions of amino acid residues which substantially do not impair the activity or properties of the sequences described herein, such as those predicted by homology software e.g. BLOSUM62 matrices.

### AAV Particles

The rAAV particles with the insertion sequences described herein have an increased transduction enrichment in the brain, heart, or brain and heart. In some instances, the increased transduction enrichment comprises a 1-fold, 2-fold, 3-fold, 4-fold, 5-fold, 6-fold, 7-fold, 8-fold, 9-fold or 10-fold increase, or more. In some instances, the increased transduction enrichment is at least 1-fold. In some instances, the increased transduction enrichment is at least 2-fold. In some instances, the increased transduction enrichment is at least 4-fold.

The rAAV particles with the insertion sequences described herein have an increased expression enrichment in the brain. Detecting whether a rAAV possesses more or less specificity for a target in vivo environment, includes measuring a level of gene expression product (e.g., RNA or protein) expressed from the heterologous nucleic acid encapsidated by the rAAV in a tissue sample obtained from a subject. Suitable methods for measuring expression of a gene expression product include next-generation sequencing (NGS) and quantitative polymerase chain reaction (qPCR).

The increased expression in the brain, heart, or brain and heart is represented by the enrichment values provided in Tables 1-3

### Heterologous Nucleic Acids

Disclosed herein are therapeutic nucleic acids useful for the treatment or prevention of a disease or condition, or symptom of the disease or condition. In some embodiments, the therapeutic nucleic acids encode a therapeutic gene expression product. Non-limiting examples of gene expression products include proteins, polypeptides, peptides, enzymes, antibodies, antigen binding fragments, nucleic acid (RNA, DNA, antisense oligonucleotide, siRNA, and the like), and gene editing components, for use in the treatment, prophylaxis, and/or amelioration of the disease or disorder, or symptoms of the disease or disorder. In some instances, the therapeutic nucleic acids are placed in an organism, cell, tissue or organ of a subject by way of a rAAV, such as those disclosed herein.

Disclosed herein are rAAVs, each comprising a viral vector (e.g., a single stranded DNA molecule (ssDNA)). In some instances, the viral vector comprises two inverted terminal repeat (ITR) sequences that are about 145 bases each, flanking a transgene. In some embodiments, the transgene comprises a therapeutic nucleic acid, and in some cases, a promoter in cis with the therapeutic nucleic acid in an open reading frame (ORF). The promoter is capable of initiating transcription of therapeutic nucleic acid in the nucleus of the target cell. The ITR sequences can be from any AAV serotype. Non-limiting examples of AAV serotypes include AAV1, AAV2, AAV3, AAV4, AAV5, AAV6, AAV7, AAV8, AAV9, AAV10, AAV11, and AAV12. In some cases, an ITR is from AAV2. In some cases, an ITR is from AAV9.

Disclosed herein are transgenes that can comprise any number of nucleotides. In some cases, a transgene can comprise less than about 100 nucleotides. In some cases, a transgene can comprise at least about 100 nucleotides. In some cases, a transgene can comprise at least about 200 nucleotides. In some cases, a transgene can comprise at least about 300 nucleotides. In some cases, a transgene can comprise at least about 400 nucleotides. In some cases, a transgene can comprise at least about 500 nucleotides. In some cases, a transgene can comprise at least about 1000 nucleotides. In some cases, a transgene can comprise at least about 5000 nucleotides. In some cases, a transgene can comprise over 5,000 nucleotides. In some cases, a transgene can comprise between about 500 and about 5000 nucleotides. In some cases, a transgene comprises about 5000 nucleotides. In any of the cases disclosed herein, the transgene can comprise DNA, RNA, or a hybrid of DNA and RNA. In some cases, the transgene can be single stranded. In some cases, the transgene can be double stranded.

Disclosed herein are transgenes useful for modulating the expression or activity of a target gene or gene expression product thereof. In some instances, the transgene is encapsidated by an rAAV capsid protein of an rAAV particle described herein. In some instances, the rAAV particle is delivered to a subject to treat a disease or condition disclosed herein in the subject. In some instances, the delivery is systemic.

The transgenes disclosed herein are useful for expressing an endogenous gene at a level similar to that of a healthy or normal individual. This is particularly useful in the treatment of a disease or condition related to the underexpression, or lack of expression, of a gene expression product. In some embodiments, the transgenes disclosed herein are useful for overexpressing an endogenous gene, such that an expression level of the endogenous gene is above the expression level of a healthy or normal individual. Additionally, transgenes can be used to express exogenous genes (e.g., active agent such as an antibody, peptide, nucleic acid, or gene editing components). In some embodiments, the therapeutic gene expression product is capable of altering, enhancing, increasing, or inducing the activity of one or more endogenous biological processes in the cell. In some embodiments, the transgenes disclosed herein are useful for reducing expression of an endogenous gene, for example, a dominant negative gene. In some embodiments, the therapeutic gene expression product is capable of altering, inhibiting, reducing, preventing, eliminating, or impairing the activity of one or more endogenous biological processes in the cell. In some aspects, the increase of gene expression refers to an increase by at least about 20%, 30%, 40%, 50%, 60%, 70%, 80%, 85%, 90%, 95% and 100%. In certain aspects, the protein product of the targeted gene may be increased by at least about 20%, 30%, 40%, 50%, 60%, 70%, 80%, 85%, 90%, 95% and 100%. In some aspects, the decrease of gene expression refers to an increase by at least about 20%, 30%, 40%, 50%, 60%, 70%, 80%, 85%, 90%, 95% and 100%. In certain aspects, the protein product of the targeted gene may be decreased by at least about 20%, 30%, 40%, 50%, 60%, 70%, 80%, 85%, 90%, 95% and 100%.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

A transgene can be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences of the endogenous gene) is integrated into any endogenous locus, for example a safe-harbor locus. For example, a Frataxin (FXN) transgene can be inserted into an endogenous FXN gene. A transgene can be inserted into any gene, e.g., the genes as described herein.

At least one advantage of the present disclosure is that virtually any therapeutic nucleic acid may be used to express any therapeutic gene expression product. In some instances, the therapeutic gene expression product is a therapeutic protein or a peptide (e.g., antibody, antigen-binding fragment, peptide, or protein). In one embodiment the protein encoded by the therapeutic nucleic acid is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length. In some embodiments the peptide encoded is between 4-50 amino acids in length. In some embodiments, the protein encoded is a tetrapeptide, a pentapeptide, a hexapeptide, a heptapeptide, an octapeptide, a nonapeptide, or a decapeptide. In some embodiments, the protein encoded comprises a peptide of 2-30 amino acids, such as for example 5-30, 10-30, 2-25, 5-25, 10-25, or 10-20 amino acids. In some embodiments, the protein encoded comprises a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

Non-limiting examples of therapeutic protein or peptides include an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphoring, a semaphorin receptor, a serotonin transport protein, a serotonin uptake inhibitor, a serotonin receptor, a serpin, a serpin receptor, and a tumor suppressor. In certain embodiments, the therapeutic protein or peptide is selected from brain-derived neurotrophic factor (BDNF), ciliary neurotrophic factor (CNTF), macrophage colony-stimulating factor (CSF), epidermal growth factor (EGF), fibroblast growth factor (FGF), gonadotropin, interferon-gamma (IFN), insulin-like growth factor 1 (IFG-1), nerve growth factor (NGF), platelet-derived growth factor (PDGF), pigment epithelium-derived factor (PEDF), transforming growth factor (TGF), transforming growth factor-beta (TGF-B), tumor necrosis factor (TNF), vascular endothelial growth factor (VEGF), prolactin, somatotropin, X-linked inhibitor of apoptosis protein 1 (XIAP1), interleukin 1 (IL-1), IL-2, IL-3, IL-4, IL-5, IL-6, IL-7, IL-8, IL-9, IL-10, IL-10, viral IL-10, IL-11, IL-12, IL-13, IL-14, IL-15, IL-16, IL-17, and IL-18.

A therapeutic gene expression product can comprise gene editing components. Non-limiting examples of gene editing components include those required for CRISPR/Cas, artificial site-specific RNA endonuclease (ASRE), zinc finger endonuclease (ZFN), and transcription factor like effector nuclease (TALEN). In a non-limiting example, a subject having Huntington's disease is identified. The subject is then systemically administered a first amount of a rAAV encapsidating a viral vector encoding ZFN engineered to represses the transcription of the Huntingtin (HTT) gene. The rAAV will include a modified AAV capsid protein that includes an amino acid sequence provided in any one of Tables 1-3, so as to allow proper targeting of the ZFN to the brain and/or heart system. If needed, the subject is administered a second or third dose of the rAAV, until a therapeutically effective amount of the ZFN is expressed in the subject's nervous system.

A therapeutic nucleic acid can comprise a non-protein coding gene e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, rRNA, tmRNA, piRNA, double stranded RNA, snRNA, snoRNA, and/or long non-coding RNA (lncRNA). In some cases, the non-protein coding gene can modulate the expression or the activity of a target gene or gene expression product. For example, the RNAs described herein may be used to inhibit gene expression in the brain, heart, or brain and heart. In some cases, inhibition of gene expression refers to an inhibition by at least about 20%, 30%, 40%, 50%, 60%, 70%, 80%, 85%, 90%, 95% and 100%. In some cases, the protein product of the targeted gene may be inhibited by at least about 20%, 30%, 40%, 50%, 60%, 70%, 80%, 85%, 90%, 95% and 100%. The gene can be either a wild type gene or a gene with at least one mutation. The targeted protein may be either a wild type protein or a protein with at least one mutation.

A therapeutic nucleic acid can modulate the expression or activity of a gene or gene expression product expressed from the gene that is implicated in a disease or disorder of the brain, heart, or brain and heart. For example, the therapeutic nucleic acid, in some cases is a gene or a modified version of the gene described herein. In some instances, the gene or gene expression product is inhibited. In some instances, the gene or gene expression product is enhanced.

In another example, the therapeutic nucleic acid comprises an effector gene expression product such as a gene editing component specific to target a gene therein. Non-limited examples of genes include target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11B, PEX12, PEX13, PEX14, PEX16, PEX19, and PEX26. In some instances, the gene or gene expression product is inhibited. In some instances, the gene or gene expression product is enhanced.

### AAV Vectors

Aspects disclosed herein comprise plasmid vectors comprising a nucleic acid sequence encoding the AAV capsids and AAV capsid proteins described herein. AAV vectors described herein are useful for the assembly of a rAAV and viral packaging of a heterologous nucleic acid. In addition, an AAV vector may encode a transgene comprising the heterologous nucleic acid.

An AAV vector can comprise a transgene, which in some cases encodes a heterologous gene expression product (e.g., therapeutic gene expression product, recombinant capsid protein, and the like). The transgene is in cis with two inverted terminal repeats (ITRs) flanking the transgene. The transgene may comprise a therapeutic nucleic acid encoding a therapeutic gene expression product. Due to the limited packaging capacity of the rAAV (˜5 KB), in some cases, a longer transgene may be split between two AAV vectors, the first with 3′ splice donor and the second with a 5′ splice acceptor. Upon co-infection of a cell, concatemers form, which are spliced together to express a full-length transgene.

A transgene is generally inserted so that its expression is driven by the endogenous promoter at the integration site, namely the promoter that drives expression of the endogenous gene into which a transgene is inserted. In some instances, a transgene comprises a promoter and/or enhancer, for example a constitutive promoter or an inducible or tissue/cell specific promoter. As a non-limiting example, the promoter may be CMV promoter, a CMV-β-Actin-intron-β-Globin hybrid promoter (CAG), CBA promoter, FRDA or FXN promoter, UBC promoter, GUSB promoter, NSE promoter, Synapsin promoter, MeCP2 promoter, GFAP promoter, H1 promoter, U6 promoter, NFL promoter, NFH promoter, SCN8A promoter, or PGK promoter. As a non-limiting example, promoters can be tissue-specific expression elements include, but are not limited to, human elongation factor 1α-subunit (EF1α), immediate-early cytomegalovirus (CMV), chicken β-actin (CBA) and its derivative CAG, the β glucuronidase (GUSB), and ubiquitin C (UBC). The transgene may include a tissue-specific expression elements for neurons such as, but not limited to, neuron-specific enolase (NSE), platelet-derived growth factor (PDGF), platelet-derived growth factor B-chain (PDGF-β), the synapsin (Syn), the methyl-CpG binding protein 2 (MeCP2), Ca2+/calmodulin-dependent protein kinase II (CaMKII), metabotropic glutamate receptor 2 (mGluR2), NFL, NFH, np32, PPE, Enk and EAAT2 promoters. The transgene may comprise a tissue-specific expression element for astrocytes such as, but not limited to, the glial fibrillary acidic protein (GFAP) and EAAT2 promoters. The transgene may comprise tissue-specific expression elements for oligodendrocytes such as, but not limited to, the myelin basic protein (MBP) promoter.

In some embodiments, the promoter is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. The promoter may provide expression of the therapeutic gene expression product for a period of time in targeted tissues such as, but not limited to, the brain, heart, or brain and heart. Expression of the therapeutic gene expression product may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

An AAV vector can comprise a genome of a helper virus. Helper virus proteins are required for the assembly of a recombinant AAV (rAAV), and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination.

The target gene or gene expression product for use in a transgene can be selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11B, PEX12, PEX13, PEX14, PEX16, PEX19, and PEX26.

An AAV vector can comprise a viral genome comprising a nucleic acid encoding the recombinant AAV (rAAV) capsid protein described herein. The viral genome can comprise a Replication (Rep) gene encoding a Rep protein, and Capsid (Cap) gene encoding an AAP protein in the first open reading frame (ORF1) or a Cap protein in the second open reading frame (ORF2). The Rep protein is selected from Rep78, Rep68, Rep52, and Rep40. In some instances, the Cap gene is modified encoding a modified AAV capsid protein described herein. A wild-type Cap gene encodes three proteins, VP1, VP2, and VP3. In some cases, VP1 is modified. In some cases, VP2 is modified. In some cases, VP3 is modified. In some cases, all three VP1-VP3 are modified. The AAV vector can comprise nucleic acids encoding wild-type Rep78, Rep68, Rep52, Rep40 and AAP proteins.

Methods of Producing rAAVs

Disclosed herein are methods of producing the AAV capsids comprising the AAV capsid proteins and viral vector encoding a therapeutic nucleic acid. The AAV capsid proteins are produced by introducing into a cell (e.g., immortalized stem cell) a first vector containing a transgene cassette flanked by inverted terminal repeat (ITR) sequences from a parental AAV virus (the transgene cassette has a promoter sequence that drives transcription of a heterologous nucleic acid in the nucleus of the target cell), a second vector encoding the AAV genome with a AAV capsid protein (encoding the AAV Rep gene as well as the modified Cap gene for the variant being produced), and a third vector encoding helper virus proteins, required for assembly of the AAV capsid structure and packaging of the transgene in the modified AAV capsid structure. The assembled AAV capsid can be isolated and purified from the cell using suitable methods known in the art.

The transgenes contained in a recombinant AAV (rAAV) vector and encapsidated by the AAV capsid proteins of the present disclosure are also provided herein. The transgenes disclosed herein are delivered to a subject for a variety of purposes, such as to treat a disease or condition in the subject. The transgene can be gene editing components that modulate the activity or expression of a target gene or gene expression product. Alternatively, the transgene is a gene encoding a therapeutic gene expression product that is effective to modulate the activity or expression of itself, or another target gene or gene expression product.

Aspects disclosed herein provide methods of manufacturing rAAV virus or virus particles comprising: (a) introducing into a cell a nucleic acid comprising: (i) first vector containing a transgene cassette flanked by inverted terminal repeat (ITR) sequences from a parental AAV virus (the transgene cassette has a promoter sequence that drives transcription of a heterologous nucleic acid in the nucleus of the target cell); (ii) a second vector encoding the AAV genome with a AAV capsid protein of the present invention; and (iii) a vector encoding helper virus proteins, required for assembly of the AAV capsid structure and packaging of the transgene in the modified AAV capsid structure; (b) expressing in the cell the AAV capsid protein described herein; (c) assembling an AAV particle comprising the AAV capsid proteins disclosed herein; and (d) packaging the AAV particle. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the Cap gene is derived from the deoxyribose nucleic acid (DNA) provided in SEQ ID NO: 6. In some instances, the 5′ ITR and the 3′ ITR are derived from an AAV2 serotype. In some instances, the 5′ ITR and the 3′ ITR are derived from an AAV5 serotype. In some instances, the 5′ ITR and the 3′ ITR are derived from an AAV9 serotype. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The Cap gene disclosed here comprises any one of SEQ ID NOS: 57-73 from Table 4, which are DNA sequences encoding the modified AAV capsid protein portions of the present disclosure.

In some instances, the methods comprise packing the first nucleic acid sequence encoding the therapeutic gene expression product such that it becomes encapsidated by the modified AAV capsid protein. In some embodiments, the rAAV particles are isolated, concentrated, and purified using suitable viral purification methods, such as those described herein.

In some cases, rAAVs of the present disclosure are generated using the methods described in Challis, R. C. et al. Nat. Protoc. 14, 379 (2019). Briefly, triple transfection of HEK293T cells (ATCC) using polyethylenimine (PEI) is performed, viruses are collected after 120 hours from both cell lysates and media and purified over iodixanol. In a non-limiting example, the rAAVs are generated by triple transfection of precursor cells (e.g., HEK293T) cells using a standard transfection protocol (e.g., PEI). Viral particles are harvested from the media after a period of time (e.g., 72 h post transfection) and from the cells and media at a later point in time (e.g., 120 h post transfection). Virus present in the media is concentrated by precipitation with 8% polyethylene glycol (PEG) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNaseI-resistant vector genome copies (VGs) using qPCR and the linearized genome plasmid as a control.

The cell can be selected from a human, a primate, a murine, a feline, a canine, a porcine, an ovine, a bovine, an equine, an epine, a caprine and a lupine host cell. In some instances, the cell is a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some cases, the immortalized cell is a HEK293cell. In some instances, the cell is a differentiated cell. Based on the disclosure provided, it is expected that this system can be used in conjunction with any transgenic line expressing a recombinase in the target cell type of interest to develop AAV capsids that more efficiently transduce that target cell population.

### Methods of Treatment

Disclosed herein are methods of treating a disease or condition, or a symptom of the disease or condition, in a subject, comprising administrating of therapeutically effective amount of one or more compositions (e.g., rAAV particle, AAV vector, pharmaceutical composition) disclosed herein to the subject. In some embodiments, the composition is a rAAV capsid protein described herein. In some embodiments, the composition is an isolated and purified rAAV capsid protein described herein. In some embodiments, the rAAV particle encapsidates an AAV vector comprising a transgene (e.g., therapeutic nucleic acid). In some embodiments, the composition is a rAAV capsid protein described herein conjugated with a therapeutic agent disclosed herein. In some embodiments, the composition is a pharmaceutical composition comprising the rAAV particle and a pharmaceutically acceptable carrier. In some embodiments, the one or more compositions are administered to the subject alone (e.g., stand-alone therapy). In some embodiments, the composition is a first-line therapy for the disease or condition. In some embodiments, the composition is a second-line, third-line, or fourth-line therapy, for the disease or condition.

Recombinant adeno-associated virus (rAAV) mediated gene delivery leverages the AAV mechanism of viral transduction for nuclear expression of an episomal heterologous nucleic acid (e.g., a transgene, therapeutic nucleic acid). Upon delivery to a host in vivo environment, a rAAV will (1) bind or attach to cellular surface receptors on the target cell, (2) endocytose, (3) traffic to the nucleus, (4) uncoat the virus to release the encapsidated heterologous nucleic acid, (5) convert of the heterologous nucleic acid from single-stranded to double-stranded DNA as a template for transcription in the nucleus, and (6) transcribe of the episomal heterologous nucleic acid in the nucleus of the host cell (“transduction”). rAAVs engineered to have an increased transduction enrichment (transcription of the episomal heterologous nucleic acid in the host cell) are desirable for gene therapy applications.

Aspects disclosed herein provide methods of treating a disease or condition in a subject, the method comprising administering to the subject a therapeutically effective amount of the rAAV of the present disclosure, or the pharmaceutical formulation of the present disclosure, wherein the gene product is a therapeutic gene product. In some embodiments, the administering is by intracranial, intraventricular, intracerebroventricular, intravenous, intraarterial, intranasal, intrathecal, intracisternae magna, or subcutaneous.

Provided here, are methods of treating a disease or a condition associated with an aberrant expression or activity of a target gene or gene expression product thereof, the method comprising modulating the expression or the activity of a target gene or gene expression product in a subject by administering a rAAV encapsidating a heterologous nucleic acid of the present disclosure. In some instances, the expression or the activity of the target gene or gene expression product is decreased, relative to that in a normal (non-diseased) individual; and administering the rAAV to the subject is sufficient to increase the expression of the activity of the target gene or gene expression product. In some instances, the expression or the activity of the gene or gene expression product is increased, relative to that in a normal individual; and administering the rAAV to the subject is sufficient to decrease the expression or the activity of the target gene or gene expression product. In a non-limiting example, a subject diagnosed with Alzheimer's disease, which is caused, in some cases, by a gain-of-function of a Presenilin 1 and/or Presenilin 2 (encoded by the gene PSEN1 and PSEN2, respectively) is administered a rAAV disclosed herein encapsidating a therapeutic nucleic acid that is a silencing RNA (siRNA), or other RNAi with a loss-of-function effect on PSEN1 mRNA.

Also provided are methods of preventing a disease or condition disclosed herein in a subject comprising administering to the subject a therapeutically effective amount of an rAAV vector comprising a nucleic acid sequence encoding a therapeutic gene expression product described herein. The rAAV vector may be encapsidated in the modified capsid protein or rAAV viral particle described herein. In some instances, the therapeutic gene expression product is effective to modulate the activity or expression of a target gene or gene expression product.

Disclosed herein are methods of treating a disease or condition in a subject by administering a composition comprising a rAAV disclosed herein. An advantage of the rAAVs disclosed herein, is that the rAAV may be used to treat virtually any disease or condition that would benefit from a transgene therapy, including but not limited to spinal muscular atrophy (SMA), amyotrophic lateral sclerosis (ALS), Parkinson's disease, Pompe disease, mucopolysaccharidosis type II, fragile X syndrome, STXBP1 encephalopathy, Krabbe disease, Huntington's disease, Alzheimer's disease, Battens disease, lysosomal storage disorders, glioblastoma multiforme, Rett syndrome, Leber's congenital amaurosis, Late infantile neuronal ceroid lipofuscinosis (LINCL), chronic pain, stroke, spinal cord injury, traumatic brain injury and lysosomal storage disorders.

In some cases, the disease or condition is localized to a particular in vivo environment in the subject, e.g., the brain, heart, or brain and heart. The compositions of the present disclosure are particularly useful for the treatment of the diseases or conditions described herein because they specifically or more efficiently target the in vivo environment and deliver a therapeutic nucleic acid engineered to modulate the activity or the expression of a target gene expression product involved with the pathogenesis or pathology of the disease or condition.

Provided herein are methods of treating a disease or a condition, or a symptom of the disease or condition, in a subject, comprising: (a) diagnosing a subject with a disease or a condition affecting a target in vivo environment; and (b) treating the disease or the condition by administering to the subject a therapeutically effective amount of a composition disclosed herein (e.g., rAAV particle, AAV vector, pharmaceutical composition), wherein the composition is engineered with an increased specificity for the target in vivo environment.

Disclosed herein are methods of treating a disease or a condition, or a symptom of the disease or the condition, afflicting a target in a subject comprising: (a) administering to the subject a composition (e.g., rAAV particle, AAV vector, pharmaceutical composition); and (b) expressing the therapeutic nucleic acid into a target in vivo environment in the subject with an increased transduction enrichment.

In some embodiments, methods of treating a disease or condition affecting the brain, heart, or brain and heart comprise administering a rAAV particle to a brain, heart, or brain and heart, respectively, in a subject, the rAAV particle comprising an rAAV capsid protein comprising an insertion of about, five, six, or seven amino acids of an amino acid sequence provided in Tables 1-3 at an amino acid position 588-589 in a parental AAV capsid protein. In some embodiments, methods of treating a disease or condition affecting the brain, heart, or brain and heart comprise administering a rAAV particle to a brain, heart, or brain and heart in a subject, respectively, the rAAV particle comprising an rAAV capsid protein comprising an insertion of about, five, six, or seven amino acids of an amino acid sequence as well as one or more substitution at amino acid found at amino acid positions 587-590 [AQAQ] such as provided in Tables 1-3. In some embodiments, the parental AAV capsid protein is AAV9 capsid protein (for e.g., provided in SEQ ID NO: 1.

Also provided are methods of modulating a target gene expression product, the methods comprising administering to a subject in need thereof a composition (e.g., rAAV particle, AAV vector, pharmaceutical composition) disclosed herein. For example, methods provided herein comprise administering to a subject a rAAV with a rAAV capsid protein encapsidating a viral vector comprising a heterologous nucleic acid that modulates the expression or the activity of the target gene expression product.

The term “normal individual” refers to an individual that is not afflicted with the disease or the condition characterized by the variation in expression or activity of the gene or gene expression product thereof.

In some embodiments, the disease or condition of the brain selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Charcot-Marie-Tooth syndrome, classical rhizomelic chondrodysplasia punctata (RCDP), Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, Deafness, De Morsier's Syndrome, Dejerine-Klumpke Palsy, Dementia, Dementia-Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Duchenne muscular dystrophy, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia (FTD), Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, glioblastoma, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease (IRD), Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus-Neurological Sequelae, Lyme Disease-Neurological Complications, Machado-Joseph Disease, Macrencephaly, Maple syrup urine disease, Megalencephaly, Melkersson-Rosenthal Syndrome, Meningitis, Meningitis and Encephalitis, Menkes Disease, Menkes syndrome, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Mucopolysaccharidosis II, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Myotonic dystrophy, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phenylketonuria, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Prader-Willi syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome. Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar ataxia, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, STXBP1 encephalopathy. Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tangier disease, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central Nervous Systems, Von Economo's Disease, Von Hippel-Lindau Disease (VHL), Von Hippel-Lindau syndrome, Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy and Zellweger syndrome.

In some embodiments, the pharmaceutical formulation comprises a therapeutic nucleic acid encoding a therapeutic gene expression product. In some instances, the therapeutic gene expression product is effective to modulate an activity or an expression of a target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11B, PEX12, PEX13, PEX14, PEX16, PEX19, and PEX26.

In some aspects, other examples of genes involved in neurologic or brain diseases or disorders include MAPT, IDUA, SNCA, ATXN2, Ube3a, GNS, HGSNAT, NAGLU, SGSH, CLN1, CLN3, CLN4, CLN5, CLN6, CLN7, CLN8, CTSD, ABCD1, HEXA, HEXB, ASM, ASPA, GLB1, AADC, MFN2, GNAO1, SYNGAP1, GRIN2A, GRIN2B, KCNQ2, EPM2A, NHLRC1, SLC6A1, SLC13A5, SURF1, GBE1, ATXN1, ATXN3, and ATXN7.

In some embodiments, the disease or condition of the heart includes any disease of the cardiopulmonary system, including, but not limited to heart failure, ischemia, arrhythmia, myocardial infarction, congestive heart failure, transplant rejection, abnormal heart contractility, non-ischaemic cardiomyopathy, mitral valve regurgitation, refractory myocardial ischaemia, non-ischaemic heart failure, aortic stenosis or regurgitation, abnormal Ca2+ metabolism, dysregulation of a heart-specific protein or a protein effective in modulating cardiac activity or physiology.

In some embodiments, genes involved in heart disease include those the express proteins involved in the regulation of calcium cycling in cardiomyocytes, such as a sarcoplasmic endoplasmic reticulum Ca2+ ATPase pump.

In some embodiments, cells of the heart or heart tissue comprise tissue or cells of any portion of the cardiopulmonary system. In certain aspects, cells of the heart or heart tissue comprises cardiac muscle cells/tissue, cells/tissue of the cardiac vasculature, and cells/tissue present in a cardiac valve. Cells of the heart may include cardiomyocytes, epithelial cells, endothelial cells, fibroblasts, cells of the conducting tissue, cardiac pacemaking cells, and neurons.

In some instances, the therapeutic gene expression product comprises gene editing components. In some instances, the gene editing components are selected from an artificial site-specific RNA endonuclease (ASRE), a zinc finger endonuclease (ZFN), a transcription factor like effector nuclease (TALEN), a clustered regularly interspaced short palindromic repeats (CRISPR)/Cas enzyme, and a CRISPR)/Cas guide RNA.

In some instances, the expression of a gene or expression or activity of a gene expression product is inhibited by the administration of the composition to the subject. In some instances, the expression of a gene or the expression or the activity of a gene expression product is enhanced by the administration of the composition to the subject.

### Formulations, Dosages, and Routes of Administration

Disclosed herein are methods comprising delivering a rAAV particle encapsidating a heterologous nucleic acid to the brain, heart, or brain and heart in a subject, the rAAV particle comprising (i) an increased transduction of the heterologous nucleic acid in the brain, heart, or brain and heart, wherein the rAAV particle has an rAAV capsid protein comprising an insertion of five, six, or seven amino acids of an amino acid sequence provided in Tables 1-3, at an amino acid position 588-589 in a parental AAV capsid protein as well as one or more substitution at amino acid found at amino acid positions 587-590 [AQAQ] such as provided in Tables 1-3. In various embodiments, the rAAV capsid protein may comprise one or more substitutions at amino acid positions 452-458 alone or in combination with the modifications above.

In general, methods disclosed herein comprise administering a therapeutic rAAV composition by systemic administration. In some instances, methods comprise administering a therapeutic rAAV composition by intravenous (“i.v.”) administration. One may administer therapeutic rAAV compositions by additional routes, such as subcutaneous injection, intramuscular injection, intradermal injection, transdermal injection, percutaneous administration, intranasal administration, intralymphatic injection, rectal administration intragastric administration, intraocular administration, intracerebroventricular administration, intrathecally, intracisternal, or any other suitable parenteral administration. Routes, dosage, time points, and duration of administrating therapeutics may be adjusted. In some embodiments, administration of therapeutics is prior to, or after, onset of either, or both, acute and chronic symptoms of the disease or condition. Other routes of delivery to the brain, heart, or brain and heart include, but are not limited to intracranial administration, lateral cerebroventricular administration, and endovascular administration.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some cases, a dose of the pharmaceutical composition may comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, or 2×10¹⁷. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some cases, the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, or 4×10¹⁷. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some cases, the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, or 8×10¹⁷. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, or 9×10¹⁷.

Disclosed herein, in some embodiments are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the pharmaceutical forms of the rAAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

In some cases, for administration of an injectable aqueous solution, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous or intrathecal administration.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of rAAV compositions and time of administration of such compositions will be within the purview of the skilled artisan having benefit of the present teachings. It is likely, however, that the administration of therapeutically-effective amounts of the disclosed compositions may be achieved by a single administration, for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In certain embodiments, the data obtained from cell culture assays and animal studies are used in formulating the therapeutically effective daily dosage range and/or the therapeutically effective unit dosage amount for use in mammals, including humans. In certain embodiments, the dosage range and/or the unit dosage amount varies within this range depending upon the dosage form employed and the route of administration utilized.

### Combination Therapies

A therapeutic rAAV may be used alone or in combination with an additional therapeutic agent (together, “therapeutic agents”). In some cases, a therapeutic rAAV as used herein is administered alone. The therapeutic agent may be administered together or sequentially in a combination therapy. The combination therapy may be administered within the same day, or may be administered one or more days, weeks, months, or years apart.

The additional therapeutic agent can comprise a small molecule. The additional therapeutic agent can comprise an antibody, or antigen-binding fragment. The additional therapeutic agent can include lipid nanoparticle-based therapies, anti-sense oligonucleotide therapies, as well as other viral therapies.

The additional therapeutic agent can comprise a cell-based therapy. Exemplary cell-based therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. The NK cells and CAR-NK cells can comprise a cytokine receptor and a suicide gene. The cell-based therapy can comprise a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### Kits

Disclosed herein are kits comprising compositions disclosed herein. Also disclosed herein are kits for the treatment or prevention of a disease or conditions of the brain, heart, or brain and heart. In some instances, the disease or condition is cancer, a pathogen infection, pulmonary disease or condition, neurological disease, muscular disease, or an immune disorder, such as those described herein.

In one embodiment, a kit can include a therapeutic or prophylactic composition containing an effective amount of a composition of a rAAV particle encapsidating a recombinant AAV vector encoding a therapeutic nucleic acid (e.g., therapeutic nucleic acid) and a recombinant AAV (rAAV) capsid protein of the present disclosure. In another embodiment, a kit can include a therapeutic or prophylactic composition containing an effective amount of cells modified by the TAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some instances, the kit further comprises a cell. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the kit further comprises an AAV vector comprising a heterologous nucleic acid encoding a therapeutic gene expression product. In some instances, the AAV vector is an episome.

In some cases, rAAV are provided together with instructions for administering the rAAV to a subject having or at risk of developing the disease or condition (e.g., disease of the brain, heart, or brain and heart). Instructions can generally include information about the use of the composition for the treatment or prevention of the disease or condition.

In some cases, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some cases, instructions provide procedures for administering the rAAV to the subject alone. In some instances, the instructions provide that the rAA Vis formulated for systemic delivery.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject” is any organism. In some instances, the organism is a mammal. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In certain aspects, the mammal is a human. The term “animal” as used herein comprises human beings and non-human animals. In one embodiment, a “non-human animal” is a mammal, for example a rodent such as rat or a mouse. In one embodiment, a “non-human primate” is a mammal, for example a monkey. In some instances, the subject is a patient, which as used herein, may refer to a subject diagnosed with a particular disease or disorder.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “adeno-associated virus,” or “AAV” as used herein refers to the adeno-associated virus or derivatives thereof. Non-limited examples of AAV's include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wildtype, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a transgene, therapeutic nucleic acid, viral genome). In some instances, the AAV capsid of the instant disclosure is a modified AAV capsid, relative to a corresponding parental AAV capsid protein.

The term “tropism” as used herein refers to a quality or characteristic of the AAV capsid that may include specificity for, and/or an increase or a decrease in enrichment of, expressing the encapsidated genetic information into an in vivo environment, relative to a second in vivo environment. An in vivo environment, in some instances, is a cell-type. An in vivo environment, in some instances, is an organ or organ system.

The term “AAV vector” as used herein refers to nucleic acid polymer encoding genetic information related to the virus. The AAV vector may be a recombinant AAV vector (rAAV), which refers to an AAV vector generated using recombinatorial genetics methods. In some instances, the rAAV vector comprises at least one heterologous polynucleotide (e.g. a polynucleotide other than a wild-type or naturally occurring AAV genome such as a transgene).

The term “AAV particle” as used herein refers to an AAV virus, virion, AAV capsid protein or component thereof. In some cases, the AAV particle is modified relative to a parental AAV particle.

The term “gene product” of “gene expression product” refers to an expression product of a polynucleotide sequence such as, for e.g., a polypeptide, peptide, protein or RNA, including interfering RNA (e.g., siRNA, miRNA, shRNA) and messenger RNA (mRNA).

The term “heterologous” as used herein refers to a genetic element (e.g., coding region) or gene expression product (e.g., RNA, protein) that is derived from a genotypically distinct entity from that of the rest of the entity to which it is being compared.

The term “endogenous” as used herein refers to a genetic element (e.g., coding region) or gene expression product (e.g., RNA, protein) that is naturally occurring in or associated with an organism or a particular cell within the organism.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, systemic administration.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. Alternatively, a sample can be obtained through primary patient derived cell lines, or archived patient samples in the form of preserved samples, or fresh frozen samples.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “brain” means a tissue selected from brain, thalamus, cortex, putamen, lateral ventricles, medulla, the pons, the amygdala, the motor cortex, caudate, hypothalamus, striatum, ventral midbrain, neocortex, basal ganglia, hippocampus, cerebrum, cerebellum, brain stem, and spinal cord. The brain includes a variety of cortical and subcortical areas, including the frontal, temporal, occipital and parietal lobes.

The term “systemic delivery” is defined as a route of administration of medication or other substance into a circulatory system so that the entire body is affected. Administration can take place via enteral administration (absorption of the drug through the gastrointestinal tract) or parenteral administration (generally injection, infusion, or implantation). “Circulatory system” includes both blood or cerebrospinal fluid circulatory systems. Examples of systemic administration for the brain include intraarterial, intravenous or intrathecal injection. Other examples include administration to the cerebrospinal fluid at any location, in the spine (i.e. but not limited to lumbar) or brain (i.e. but not limited to cisterna magna). The terms “systemic administration” and “systemic delivery” are used interchangeably.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

### EXAMPLES

### Example 1

### Method of Identifying the Modified Capsid Proteins in Cynomolgus Macaques

Following IV injection, unmodified AAV9 does not have sufficient tissue enrichment to treat many human diseases that would otherwise be amenable to treatment via delivery of an AAV cargo of 4.7 kbp DNA. Directed evolution of AAV9 was performed as a means of increasing viral tissue enrichment to levels that enable disease treatment.

Adeno-Associated Virus serotype 9 (AAV9) was modified by inserting a stretch of 7 amino acids (7mer) following residue #588 in its capsid coding sequence. The 7mer was randomly generated via degenerate DNA basepairs, thus sampling all 207 possible 7mer amino acid sequences. Three batches of this library with billions of variants were prepared separately.

One batch was IV injected into three African Green Monkeys, then three weeks later the animals were sacrificed and tissue samples were screened with PCR to uncover the best 7mer sequences that caused improved enrichment relative to AAV9 in the brain and heart. The other batch was IV injected into two Cynomologus Macaque Non-Human Primates, then two weeks later the animals were sacrificed and tissue samples were screened with PCR to uncover the best 7mer sequences that caused improved enrichment relative to AAV9 in the brain and heart. The third batch was delivered identically to the second batch and data were analyzed identically, but in a separate study using different Cynomolgus Macaques.

Following these three PCR screenings of tissues, the top 41,000 variants identified in the NHP brains and hearts were brought forward into a ‘round 2’ screening. This smaller library was IV injected into three Cynomologus Macaque Non-Human Primates, then two weeks later the animals were sacrificed and tissue samples were screened with PCR to uncover the best 7mer sequences that caused further improved enrichment relative to AAV9 in the brain and heart.

What is claimed is:

1. A mutant Adeno-Associated Virus (AAV) capsid protein comprising at least 95% sequence identity to the wild-type AAV capsid protein, wherein the mutant AAV capsid protein comprises a sequence selected from the group consisting of SEQ ID NO: 2-1496.

2. The mutant AAV capsid protein of claim 1, wherein the mutant AAV capsid protein comprises 7-mer amino acid insertion sequence located between residues 588 and 589 in relation to the wild-type AAV capsid protein.

3. The mutant AAV capsid protein of claim 1, wherein the mutant AAV capsid protein comprises at least 95% sequence identity to the wild-type Adeno-Associated Virus serotype 9 (AAV9) capsid of SEQ ID NO: 1.

4. The mutant AAV capsid protein of claim 1, wherein the amino acids immediately preceding the 7-mer amino acid insertion sequence are -AQ-.

5. The mutant AAV capsid protein of claim 1, wherein the amino acids immediately following the 7-mer amino acid insertion sequence are -AQ-.

6. The mutant AAV capsid protein of claim 1, wherein 60 copies of the AAV capsid protein are assembled into the AAV capsid.

7. The mutant AAV capsid protein of claim 1, wherein the AAV capsid protein is present in VP1, VP2, and VP3 of the mutant AAV capsid.

8. The mutant AAV capsid protein of claim 1, wherein the mutant AAV capsid protein comprises a sequence selected from the group consisting of SEQ ID NO: 2-898.

9. The mutant AAV capsid protein of claim 8, further characterized by an increased transduction enrichment relative to the wild-type AAV9 when measured in brain tissue in a subject when delivered to the subject systemically.

10. The mutant AAV capsid protein of claim 9, wherein the mutant AAV capsid protein comprises a sequence selected from the group consisting of SEQ ID NO: 2-594.

11. The mutant AAV capsid protein of claim 1, wherein the mutant AAV capsid protein comprises a sequence selected from the group consisting of SEQ ID NO: 595-1496.

12. The mutant AAV capsid protein of claim 11, further characterized by an increased transduction enrichment relative to the wild-type AAV9 when measured in heart tissue in a subject when delivered to the subject systemically.

13. The mutant AAV capsid protein of claim 12, wherein the mutant AAV capsid protein comprises a sequence selected from the group consisting of SEQ ID NO: 899-1496.

14. The mutant AAV capsid protein of claim 1, wherein the mutant AAV capsid protein comprises a sequence selected from the group consisting of SEQ ID NO: 595-898.

15. The mutant AAV capsid protein of claim 14, further characterized by an increased transduction enrichment relative to the wild-type AAV9 when measured in brain tissue and heart tissue in a subject when delivered to the subject systemically.

16. An AAV capsid comprising a mutant AAV capsid protein comprising at least 95% sequence identity to the wild-type AAV capsid protein, wherein the mutant AAV capsid protein comprises a sequence selected from the group consisting of SEQ ID NO: 2-1496.

17. The AAV capsid of claim 16, wherein the AAV capsid is chimeric.

18. The AAV capsid of claim 17, wherein the capsid is isolated and purified.

19. The AAV capsid of claim 16, wherein the mutant AAV capsid protein comprises a sequence selected from the group consisting of SEQ ID NO: 2-898.

20. The AAV capsid of claim 19, further characterized by an increased transduction enrichment relative to the wild-type AAV9 when measured in brain tissue in a subject when delivered to the subject systemically.

21. The AAV capsid of claim 20, wherein the mutant AAV capsid protein comprises a sequence selected from the group consisting of SEQ ID NO: 2-594.

22. The AAV capsid of claim 16, wherein the mutant AAV capsid protein comprises a sequence selected from the group consisting of SEQ ID NO: 595-1496.

23. The AAV capsid of claim 22, further characterized by an increased transduction enrichment relative to the wild-type AAV9 when measured in heart tissue in a subject when delivered to the subject systemically.

24. The AAV capsid of claim 23, wherein the mutant AAV capsid protein comprises a sequence selected from the group consisting of SEQ ID NO: 899-1496.

25. The AAV capsid of claim 16, wherein the mutant AAV capsid protein comprises a sequence selected from the group consisting of SEQ ID NO: 595-898.

26. The AAV capsid of claim 25, further characterized by an increased transduction enrichment relative to the wild-type AAV9 when measured in brain tissue and heart tissue in a subject when delivered to the subject systemically.

27. A composition comprising an AAV capsid, wherein the AAV capsid comprises an AAV capsid protein comprising at least 95% sequence identity to the wild-type AAV capsid protein, wherein the mutant AAV capsid protein comprises a sequence selected from the group consisting of SEQ ID NO: 2-1496.

28. The composition of claim 27, formulated for systematic administration to a subject.

29. The composition of claim 28, wherein systematic administration of the composition results in expression of a gene product delivered by the AAV capsid.

30. The composition of claim 29, wherein administration of the composition results in increased transduction enrichment relative to the wild-type AAV9 when measured in heart and/or brain.

31. A method of treating a disease or condition in a subject comprising administering a therapeutically effective amount of a composition of claim 27.

32. The composition of claim 27, for use in treating a disease or condition in a subject, the use comprising administering a therapeutically effective amount of a composition of claim 27.


SEQUENCE LISTING (1496 sequences)

SEQ ID NO: 1 - protein, 736 aa, synthetic construct
  REGION 1..736: note = Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 11 aa, synthetic construct
  REGION 1..11: note = Synthetic
AQAAATAAQAQ

SEQ ID NO: 3 - protein, 11 aa, synthetic construct
  REGION 1..11: note = Synthetic
AQAALPPRSAQ

SEQ ID NO: 4 - protein, 11 aa, synthetic construct
  REGION 1..11: note = Synthetic
AQAASSGHSAQ

SEQ ID NO: 5 - protein, 11 aa, synthetic construct
  REGION 1..11: note = Synthetic
AQAESRTSAAQ

SEQ ID NO: 6 - protein, 11 aa, synthetic construct
  REGION 1..11: note = Synthetic
AQAFSVSPDAQ

SEQ ID NO: 7 - protein, 11 aa, synthetic construct
  REGION 1..11: note = Synthetic
AQAGGHSNNAQ

SEQ ID NO: 8 - protein, 11 aa, synthetic construct
  REGION 1..11: note = Synthetic
AQAGPSNAMAQ

SEQ ID NO: 9 - protein, 11 aa, synthetic construct
  REGION 1..11: note = Synthetic
AQAGSIRDDAQ

SEQ ID NO: 10 - protein, 11 aa, synthetic construct
  REGION 1..11: note = Synthetic
AQAHGDGSLAQ

SEQ ID NO: 11 - protein, 11 aa, synthetic construct
  REGION 1..11: note = Synthetic
AQAHSSGVGAQ

SEQ ID NO: 12 - protein, 11 aa, synthetic construct
  REGION 1..11: note = Synthetic
AQAHTHTLSAQ

SEQ ID NO: 13 - protein, 11 aa, synthetic construct
  REGION 1..11: note = Synthetic
AQAIAGYASAQ

SEQ ID NO: 14 - protein, 11 aa, synthetic construct
  REGION 1..11: note = Synthetic
AQAISNTTVAQ

SEQ ID NO: 15 - protein, 11 aa, synthetic construct
  REGION 1..11: note = Synthetic
AQALARDHSAQ

SEQ ID NO: 16 - protein, 11 aa, synthetic construct
  REGION 1..11: note = Synthetic
AQALRGSLCAQ

SEQ ID NO: 17 - protein, 11 aa, synthetic construct
  REGION 1..11: note = Synthetic
AQAMDRTNMAQ

SEQ ID NO: 18 - protein, 11 aa, synthetic construct
  REGION 1..11: note = Synthetic
AQAMQTRNPAQ

SEQ ID NO: 19 - protein, 11 aa, synthetic construct
  REGION 1..11: note = Synthetic
AQAMSSLTAAQ

SEQ ID NO: 20 - protein, 11 aa, synthetic construct
  REGION 1..11: note = Synthetic
AQAPGALMTAQ

SEQ ID NO: 21 - protein, 11 aa, synthetic construct
  REGION 1..11: note = Synthetic
AQAPREIAIAQ

SEQ ID NO: 22 - protein, 11 aa, synthetic construct
  REGION 1..11: note = Synthetic
AQAQADRPDAQ

SEQ ID NO: 23 - protein, 11 aa, synthetic construct
  REGION 1..11: note = Synthetic
AQAQGNLAFAQ

SEQ ID NO: 24 - protein, 11 aa, synthetic construct
  REGION 1..11: note = Synthetic
AQARSNEMTAQ

SEQ ID NO: 25 - protein, 11 aa, synthetic construct
  REGION 1..11: note = Synthetic
AQASGHAPTAQ

SEQ ID NO: 26 - protein, 11 aa, synthetic construct
  REGION 1..11: note = Synthetic
AQASGSNAHAQ

SEQ ID NO: 27 - protein, 11 aa, synthetic construct
  REGION 1..11: note = Synthetic
AQASLAVTSAQ

SEQ ID NO: 28 - protein, 11 aa, synthetic construct
  REGION 1..11: note = Synthetic
AQASRDSGSAQ

SEQ ID NO: 29 - protein, 11 aa, synthetic construct
  REGION 1..11: note = Synthetic
AQASRSESYAQ

SEQ ID NO: 30 - protein, 11 aa, synthetic construct
  REGION 1..11: note = Synthetic
AQASTRVVNAQ

SEQ ID NO: 31 - protein, 11 aa, synthetic construct
  REGION 1..11: note = Synthetic
AQATSDRPTAQ

SEQ ID NO: 32 - protein, 11 aa, synthetic construct
  REGION 1..11: note = Synthetic
AQATVTHKDAQ

SEQ ID NO: 33 - protein, 11 aa, synthetic construct
  REGION 1..11: note = Synthetic
AQAVGTMAPAQ

SEQ ID NO: 34 - protein, 11 aa, synthetic construct
  REGION 1..11: note = Synthetic
AQDASRVLAAQ

SEQ ID NO: 35 - protein, 11 aa, synthetic construct
  REGION 1..11: note = Synthetic
AQDMTGLVIAQ

SEQ ID NO: 36 - protein, 11 aa, synthetic construct
  REGION 1..11: note = Synthetic
AQDNRTRTNAQ

SEQ ID NO: 37 - protein, 11 aa, synthetic construct
  REGION 1..11: note = Synthetic
AQDPSRSASAQ

SEQ ID NO: 38 - protein, 11 aa, synthetic construct
  REGION 1..11: note = Synthetic
AQDPTTRNEAQ

SEQ ID NO: 39 - protein, 11 aa, synthetic construct
  REGION 1..11: note = Synthetic
AQDRSNITLAQ

SEQ ID NO: 40 - protein, 11 aa, synthetic construct
  REGION 1..11: note = Synthetic
AQDSAQNRGAQ

SEQ ID NO: 41 - protein, 11 aa, synthetic construct
  REGION 1..11: note = Synthetic
AQDSGPSKAAQ

SEQ ID NO: 42 - protein, 11 aa, synthetic construct
  REGION 1..11: note = Synthetic
AQDSIQYSFAQ

SEQ ID NO: 43 - protein, 11 aa, synthetic construct
  REGION 1..11: note = Synthetic
AQDSREQGRAQ

SEQ ID NO: 44 - protein, 11 aa, synthetic construct
  REGION 1..11: note = Synthetic
AQDSRLGVTAQ

SEQ ID NO: 45 - protein, 11 aa, synthetic construct
  REGION 1..11: note = Synthetic
AQDSRQPPNAQ

SEQ ID NO: 46 - protein, 11 aa, synthetic construct
  REGION 1..11: note = Synthetic
AQDSRVPSQAQ

SEQ ID NO: 47 - protein, 11 aa, synthetic construct
  REGION 1..11: note = Synthetic
AQDSVNLSSAQ

SEQ ID NO: 48 - protein, 11 aa, synthetic construct
  REGION 1..11: note = Synthetic
AQDVRHGGTAQ

SEQ ID NO: 49 - protein, 11 aa, synthetic construct
  REGION 1..11: note = Synthetic
AQDVRSNTSAQ

SEQ ID NO: 50 - protein, 11 aa, synthetic construct
  REGION 1..11: note = Synthetic
AQDVSVSAFAQ

SEQ ID NO: 51 - protein, 11 aa, synthetic construct
  REGION 1..11: note = Synthetic
AQDYAYRTGAQ

SEQ ID NO: 52 - protein, 11 aa, synthetic construct
  REGION 1..11: note = Synthetic
AQEHTRGETAQ

SEQ ID NO: 53 - protein, 11 aa, synthetic construct
  REGION 1..11: note = Synthetic
AQEITHSHSAQ

SEQ ID NO: 54 - protein, 11 aa, synthetic construct
  REGION 1..11: note = Synthetic
AQENHTFNKAQ

SEQ ID NO: 55 - protein, 11 aa, synthetic construct
  REGION 1..11: note = Synthetic
AQENRVANKAQ

SEQ ID NO: 56 - protein, 11 aa, synthetic construct
  REGION 1..11: note = Synthetic
AQEPERFMFAQ

SEQ ID NO: 57 - protein, 11 aa, synthetic construct
  REGION 1..11: note = Synthetic
AQEPRLATDAQ

SEQ ID NO: 58 - protein, 11 aa, synthetic construct
  REGION 1..11: note = Synthetic
AQERTASLHAQ

SEQ ID NO: 59 - protein, 11 aa, synthetic construct
  REGION 1..11: note = Synthetic
AQESNRHPYAQ

SEQ ID NO: 60 - protein, 11 aa, synthetic construct
  REGION 1..11: note = Synthetic
AQESSHTGGAQ

SEQ ID NO: 61 - protein, 11 aa, synthetic construct
  REGION 1..11: note = Synthetic
AQESVGRGEAQ

SEQ ID NO: 62 - protein, 11 aa, synthetic construct
  REGION 1..11: note = Synthetic
AQEVGGGSKAQ

SEQ ID NO: 63 - protein, 11 aa, synthetic construct
  REGION 1..11: note = Synthetic
AQFALPVPSAQ

SEQ ID NO: 64 - protein, 11 aa, synthetic construct
  REGION 1..11: note = Synthetic
AQFGSVQDRAQ

SEQ ID NO: 65 - protein, 11 aa, synthetic construct
  REGION 1..11: note = Synthetic
AQFGTGGVPAQ

SEQ ID NO: 66 - protein, 11 aa, synthetic construct
  REGION 1..11: note = Synthetic
AQFGTMGDKAQ

SEQ ID NO: 67 - protein, 11 aa, synthetic construct
  REGION 1..11: note = Synthetic
AQFGVTAGSAQ

SEQ ID NO: 68 - protein, 11 aa, synthetic construct
  REGION 1..11: note = Synthetic
AQFINTVPVAQ

SEQ ID NO: 69 - protein, 11 aa, synthetic construct
  REGION 1..11: note = Synthetic
AQFLVGGTVAQ

SEQ ID NO: 70 - protein, 11 aa, synthetic construct
  REGION 1..11: note = Synthetic
AQFSAFLPAAQ

SEQ ID NO: 71 - protein, 11 aa, synthetic construct
  REGION 1..11: note = Synthetic
AQFSGMSNSAQ

SEQ ID NO: 72 - protein, 11 aa, synthetic construct
  REGION 1..11: note = Synthetic
AQFVKIAKSAQ

SEQ ID NO: 73 - protein, 11 aa, synthetic construct
  REGION 1..11: note = Synthetic
AQFVTLVTSAQ

SEQ ID NO: 74 - protein, 11 aa, synthetic construct
  REGION 1..11: note = Synthetic
AQGADARLHAQ

SEQ ID NO: 75 - protein, 11 aa, synthetic construct
  REGION 1..11: note = Synthetic
AQGDAARSGAQ

SEQ ID NO: 76 - protein, 11 aa, synthetic construct
  REGION 1..11: note = Synthetic
AQGGGSHSSAQ

SEQ ID NO: 77 - protein, 11 aa, synthetic construct
  REGION 1..11: note = Synthetic
AQGHQHVTEAQ

SEQ ID NO: 78 - protein, 11 aa, synthetic construct
  REGION 1..11: note = Synthetic
AQGHSETRLAQ

SEQ ID NO: 79 - protein, 11 aa, synthetic construct
  REGION 1..11: note = Synthetic
AQGIGHMNDAQ

SEQ ID NO: 80 - protein, 11 aa, synthetic construct
  REGION 1..11: note = Synthetic
AQGLALTLNAQ

SEQ ID NO: 81 - protein, 11 aa, synthetic construct
  REGION 1..11: note = Synthetic
AQGLAVASPAQ

SEQ ID NO: 82 - protein, 11 aa, synthetic construct
  REGION 1..11: note = Synthetic
AQGLSSPLAAQ

SEQ ID NO: 83 - protein, 11 aa, synthetic construct
  REGION 1..11: note = Synthetic
AQGLVLSSTAQ

SEQ ID NO: 84 - protein, 11 aa, synthetic construct
  REGION 1..11: note = Synthetic
AQGLVSSSVAQ

SEQ ID NO: 85 - protein, 11 aa, synthetic construct
  REGION 1..11: note = Synthetic
AQGLYNIRSAQ

SEQ ID NO: 86 - protein, 11 aa, synthetic construct
  REGION 1..11: note = Synthetic
AQGNAVSVAAQ

SEQ ID NO: 87 - protein, 11 aa, synthetic construct
  REGION 1..11: note = Synthetic
AQGNHTRHAAQ

SEQ ID NO: 88 - protein, 11 aa, synthetic construct
  REGION 1..11: note = Synthetic
AQGNLTRLTAQ

SEQ ID NO: 89 - protein, 11 aa, synthetic construct
  REGION 1..11: note = Synthetic
AQGNNTRAHAQ

SEQ ID NO: 90 - protein, 11 aa, synthetic construct
  REGION 1..11: note = Synthetic
AQGNNTTSRAQ

SEQ ID NO: 91 - protein, 11 aa, synthetic construct
  REGION 1..11: note = Synthetic
AQGNQSNAAAQ

SEQ ID NO: 92 - protein, 11 aa, synthetic construct
  REGION 1..11: note = Synthetic
AQGNRTRAPAQ

SEQ ID NO: 93 - protein, 11 aa, synthetic construct
  REGION 1..11: note = Synthetic
AQGNRTTNDAQ

SEQ ID NO: 94 - protein, 11 aa, synthetic construct
  REGION 1..11: note = Synthetic
AQGNTTVNQAQ

SEQ ID NO: 95 - protein, 11 aa, synthetic construct
  REGION 1..11: note = Synthetic
AQGSDSRNSAQ

SEQ ID NO: 96 - protein, 11 aa, synthetic construct
  REGION 1..11: note = Synthetic
AQGSFTQFSAQ

SEQ ID NO: 97 - protein, 11 aa, synthetic construct
  REGION 1..11: note = Synthetic
AQGSGASVLAQ

SEQ ID NO: 98 - protein, 11 aa, synthetic construct
  REGION 1..11: note = Synthetic
AQGSGISSHAQ

SEQ ID NO: 99 - protein, 11 aa, synthetic construct
  REGION 1..11: note = Synthetic
AQGSGTPGHAQ

SEQ ID NO: 100 - protein, 11 aa, synthetic construct
  REGION 1..11: note = Synthetic
AQGSLTVLKAQ

SEQ ID NO: 101 - protein, 11 aa, synthetic construct
  REGION 1..11: note = Synthetic
AQGSMMHKDAQ

SEQ ID NO: 102 - protein, 11 aa, synthetic construct
  REGION 1..11: note = Synthetic
AQGSSRNEVAQ

SEQ ID NO: 103 - protein, 11 aa, synthetic construct
  REGION 1..11: note = Synthetic
AQGTGSERLAQ

SEQ ID NO: 104 - protein, 11 aa, synthetic construct
  REGION 1..11: note = Synthetic
AQGTQDRQLAQ

SEQ ID NO: 105 - protein, 11 aa, synthetic construct
  REGION 1..11: note = Synthetic
AQGTTLSSSAQ

SEQ ID NO: 106 - protein, 11 aa, synthetic construct
  REGION 1..11: note = Synthetic
AQGTTSHHLAQ

SEQ ID NO: 107 - protein, 11 aa, synthetic construct
  REGION 1..11: note = Synthetic
AQGVGLPTTAQ

SEQ ID NO: 108 - protein, 11 aa, synthetic construct
  REGION 1..11: note = Synthetic
AQGVGSSLSAQ

SEQ ID NO: 109 - protein, 11 aa, synthetic construct
  REGION 1..11: note = Synthetic
AQGVHASTQAQ

SEQ ID NO: 110 - protein, 11 aa, synthetic construct
  REGION 1..11: note = Synthetic
AQGVHGSNAAQ

SEQ ID NO: 111 - protein, 11 aa, synthetic construct
  REGION 1..11: note = Synthetic
AQGVISRPDAQ

SEQ ID NO: 112 - protein, 11 aa, synthetic construct
  REGION 1..11: note = Synthetic
AQGVSAVAAAQ

SEQ ID NO: 113 - protein, 11 aa, synthetic construct
  REGION 1..11: note = Synthetic
AQGVTSVVAAQ

SEQ ID NO: 114 - protein, 11 aa, synthetic construct
  REGION 1..11: note = Synthetic
AQHAGNITIAQ

SEQ ID NO: 115 - protein, 11 aa, synthetic construct
  REGION 1..11: note = Synthetic
AQHGSRSDYAQ

SEQ ID NO: 116 - protein, 11 aa, synthetic construct
  REGION 1..11: note = Synthetic
AQHGVTTQSAQ

SEQ ID NO: 117 - protein, 11 aa, synthetic construct
  REGION 1..11: note = Synthetic
AQHGVVSSSAQ

SEQ ID NO: 118 - protein, 11 aa, synthetic construct
  REGION 1..11: note = Synthetic
AQHHSPQSSAQ

SEQ ID NO: 119 - protein, 11 aa, synthetic construct
  REGION 1..11: note = Synthetic
AQHISLHQQAQ

SEQ ID NO: 120 - protein, 11 aa, synthetic construct
  REGION 1..11: note = Synthetic
AQHISSVQPAQ

SEQ ID NO: 121 - protein, 11 aa, synthetic construct
  REGION 1..11: note = Synthetic
AQHLGTVSAAQ

SEQ ID NO: 122 - protein, 11 aa, synthetic construct
  REGION 1..11: note = Synthetic
AQHMSGVNAAQ

SEQ ID NO: 123 - protein, 11 aa, synthetic construct
  REGION 1..11: note = Synthetic
AQHNVMSSFAQ

SEQ ID NO: 124 - protein, 11 aa, synthetic construct
  REGION 1..11: note = Synthetic
AQHNVSLANAQ

SEQ ID NO: 125 - protein, 11 aa, synthetic construct
  REGION 1..11: note = Synthetic
AQHQLHHGAAQ

SEQ ID NO: 126 - protein, 11 aa, synthetic construct
  REGION 1..11: note = Synthetic
AQHSSGTSGAQ

SEQ ID NO: 127 - protein, 11 aa, synthetic construct
  REGION 1..11: note = Synthetic
AQHSSSNIIAQ

SEQ ID NO: 128 - protein, 11 aa, synthetic construct
  REGION 1..11: note = Synthetic
AQHSTKAEVAQ

SEQ ID NO: 129 - protein, 11 aa, synthetic construct
  REGION 1..11: note = Synthetic
AQHTANLLDAQ

SEQ ID NO: 130 - protein, 11 aa, synthetic construct
  REGION 1..11: note = Synthetic
AQHTGASLTAQ

SEQ ID NO: 131 - protein, 11 aa, synthetic construct
  REGION 1..11: note = Synthetic
AQHTGPLSNAQ

SEQ ID NO: 132 - protein, 11 aa, synthetic construct
  REGION 1..11: note = Synthetic
AQHTLPIMSAQ

SEQ ID NO: 133 - protein, 11 aa, synthetic construct
  REGION 1..11: note = Synthetic
AQHTSTREMAQ

SEQ ID NO: 134 - protein, 11 aa, synthetic construct
  REGION 1..11: note = Synthetic
AQHTSVRIDAQ

SEQ ID NO: 135 - protein, 11 aa, synthetic construct
  REGION 1..11: note = Synthetic
AQHTTSHMEAQ

SEQ ID NO: 136 - protein, 11 aa, synthetic construct
  REGION 1..11: note = Synthetic
AQHYVSVPTAQ

SEQ ID NO: 137 - protein, 11 aa, synthetic construct
  REGION 1..11: note = Synthetic
AQIAGRNQSAQ

SEQ ID NO: 138 - protein, 11 aa, synthetic construct
  REGION 1..11: note = Synthetic
AQIAHLSSVAQ

SEQ ID NO: 139 - protein, 11 aa, synthetic construct
  REGION 1..11: note = Synthetic
AQIASSPLSAQ

SEQ ID NO: 140 - protein, 11 aa, synthetic construct
  REGION 1..11: note = Synthetic
AQIASSSLHAQ

SEQ ID NO: 141 - protein, 11 aa, synthetic construct
  REGION 1..11: note = Synthetic
AQIGGLTTPAQ

SEQ ID NO: 142 - protein, 11 aa, synthetic construct
  REGION 1..11: note = Synthetic
AQIGSLTGAAQ

SEQ ID NO: 143 - protein, 11 aa, synthetic construct
  REGION 1..11: note = Synthetic
AQIGSMNEKAQ

SEQ ID NO: 144 - protein, 11 aa, synthetic construct
  REGION 1..11: note = Synthetic
AQIGSSMPNAQ

SEQ ID NO: 145 - protein, 11 aa, synthetic construct
  REGION 1..11: note = Synthetic
AQILATGGSAQ

SEQ ID NO: 146 - protein, 11 aa, synthetic construct
  REGION 1..11: note = Synthetic
AQILDRSNMAQ

SEQ ID NO: 147 - protein, 11 aa, synthetic construct
  REGION 1..11: note = Synthetic
AQILRDGHQAQ

SEQ ID NO: 148 - protein, 11 aa, synthetic construct
  REGION 1..11: note = Synthetic
AQILSEHRVAQ

SEQ ID NO: 149 - protein, 11 aa, synthetic construct
  REGION 1..11: note = Synthetic
AQILTRSTFAQ

SEQ ID NO: 150 - protein, 11 aa, synthetic construct
  REGION 1..11: note = Synthetic
AQILVTSTSAQ

SEQ ID NO: 151 - protein, 11 aa, synthetic construct
  REGION 1..11: note = Synthetic
AQIMTSTLPAQ

SEQ ID NO: 152 - protein, 11 aa, synthetic construct
  REGION 1..11: note = Synthetic
AQINRETSFAQ

SEQ ID NO: 153 - protein, 11 aa, synthetic construct
  REGION 1..11: note = Synthetic
AQINSAGLLAQ

SEQ ID NO: 154 - protein, 11 aa, synthetic construct
  REGION 1..11: note = Synthetic
AQISAASTSAQ

SEQ ID NO: 155 - protein, 11 aa, synthetic construct
  REGION 1..11: note = Synthetic
AQISAGALIAQ

SEQ ID NO: 156 - protein, 11 aa, synthetic construct
  REGION 1..11: note = Synthetic
AQISLTNSNAQ

SEQ ID NO: 157 - protein, 11 aa, synthetic construct
  REGION 1..11: note = Synthetic
AQISSSRNGAQ

SEQ ID NO: 158 - protein, 11 aa, synthetic construct
  REGION 1..11: note = Synthetic
AQISTGKLGAQ

SEQ ID NO: 159 - protein, 11 aa, synthetic construct
  REGION 1..11: note = Synthetic
AQISTRESIAQ

SEQ ID NO: 160 - protein, 11 aa, synthetic construct
  REGION 1..11: note = Synthetic
AQITGQMSAAQ

SEQ ID NO: 161 - protein, 11 aa, synthetic construct
  REGION 1..11: note = Synthetic
AQITGQMSVAQ

SEQ ID NO: 162 - protein, 11 aa, synthetic construct
  REGION 1..11: note = Synthetic
AQITSSPTSAQ

SEQ ID NO: 163 - protein, 11 aa, synthetic construct
  REGION 1..11: note = Synthetic
AQITSSVMQAQ

SEQ ID NO: 164 - protein, 11 aa, synthetic construct
  REGION 1..11: note = Synthetic
AQITTHPTTAQ

SEQ ID NO: 165 - protein, 11 aa, synthetic construct
  REGION 1..11: note = Synthetic
AQIVAGHRDAQ

SEQ ID NO: 166 - protein, 11 aa, synthetic construct
  REGION 1..11: note = Synthetic
AQIVDTRNPAQ

SEQ ID NO: 167 - protein, 11 aa, synthetic construct
  REGION 1..11: note = Synthetic
AQIVGKPMEAQ

SEQ ID NO: 168 - protein, 11 aa, synthetic construct
  REGION 1..11: note = Synthetic
AQIVLPSTYAQ

SEQ ID NO: 169 - protein, 11 aa, synthetic construct
  REGION 1..11: note = Synthetic
AQIVSGSLGAQ

SEQ ID NO: 170 - protein, 11 aa, synthetic construct
  REGION 1..11: note = Synthetic
AQIYSSSVSAQ

SEQ ID NO: 171 - protein, 11 aa, synthetic construct
  REGION 1..11: note = Synthetic
AQKDQNRESAQ

SEQ ID NO: 172 - protein, 11 aa, synthetic construct
  REGION 1..11: note = Synthetic
AQKDSHSYNAQ

SEQ ID NO: 173 - protein, 11 aa, synthetic construct
  REGION 1..11: note = Synthetic
AQKEQQAVSAQ

SEQ ID NO: 174 - protein, 11 aa, synthetic construct
  REGION 1..11: note = Synthetic
AQKHNIPNVAQ

SEQ ID NO: 175 - protein, 11 aa, synthetic construct
  REGION 1..11: note = Synthetic
AQKPTVGFAAQ

SEQ ID NO: 176 - protein, 11 aa, synthetic construct
  REGION 1..11: note = Synthetic
AQKQAGTSVAQ

SEQ ID NO: 177 - protein, 11 aa, synthetic construct
  REGION 1..11: note = Synthetic
AQKQHSDHPAQ

SEQ ID NO: 178 - protein, 11 aa, synthetic construct
  REGION 1..11: note = Synthetic
AQKSQTNDVAQ

SEQ ID NO: 179 - protein, 11 aa, synthetic construct
  REGION 1..11: note = Synthetic
AQKSSLRFYAQ

SEQ ID NO: 180 - protein, 11 aa, synthetic construct
  REGION 1..11: note = Synthetic
AQKTSSSTVAQ

SEQ ID NO: 181 - protein, 11 aa, synthetic construct
  REGION 1..11: note = Synthetic
AQLAHQTRDAQ

SEQ ID NO: 182 - protein, 11 aa, synthetic construct
  REGION 1..11: note = Synthetic
AQLAQAASAAQ

SEQ ID NO: 183 - protein, 11 aa, synthetic construct
  REGION 1..11: note = Synthetic
AQLARTTTDAQ

SEQ ID NO: 184 - protein, 11 aa, synthetic construct
  REGION 1..11: note = Synthetic
AQLASASSVAQ

SEQ ID NO: 185 - protein, 11 aa, synthetic construct
  REGION 1..11: note = Synthetic
AQLASSTLMAQ

SEQ ID NO: 186 - protein, 11 aa, synthetic construct
  REGION 1..11: note = Synthetic
AQLDRSGSTAQ

SEQ ID NO: 187 - protein, 11 aa, synthetic construct
  REGION 1..11: note = Synthetic
AQLDTSTALAQ

SEQ ID NO: 188 - protein, 11 aa, synthetic construct
  REGION 1..11: note = Synthetic
AQLGAGPHVAQ

SEQ ID NO: 189 - protein, 11 aa, synthetic construct
  REGION 1..11: note = Synthetic
AQLGGSYGSAQ

SEQ ID NO: 190 - protein, 11 aa, synthetic construct
  REGION 1..11: note = Synthetic
AQLGSLANTAQ

SEQ ID NO: 191 - protein, 11 aa, synthetic construct
  REGION 1..11: note = Synthetic
AQLGTNRMSAQ

SEQ ID NO: 192 - protein, 11 aa, synthetic construct
  REGION 1..11: note = Synthetic
AQLGVSSVEAQ

SEQ ID NO: 193 - protein, 11 aa, synthetic construct
  REGION 1..11: note = Synthetic
AQLHNTVVSAQ

SEQ ID NO: 194 - protein, 11 aa, synthetic construct
  REGION 1..11: note = Synthetic
AQLHSPASNAQ

SEQ ID NO: 195 - protein, 11 aa, synthetic construct
  REGION 1..11: note = Synthetic
AQLHSSPSTAQ

SEQ ID NO: 196 - protein, 11 aa, synthetic construct
  REGION 1..11: note = Synthetic
AQLILTTSEAQ

SEQ ID NO: 197 - protein, 11 aa, synthetic construct
  REGION 1..11: note = Synthetic
AQLKDQGSSAQ

SEQ ID NO: 198 - protein, 11 aa, synthetic construct
  REGION 1..11: note = Synthetic
AQLLDTHYRAQ

SEQ ID NO: 199 - protein, 11 aa, synthetic construct
  REGION 1..11: note = Synthetic
AQLLDTLMQAQ

SEQ ID NO: 200 - protein, 11 aa, synthetic construct
  REGION 1..11: note = Synthetic
AQLLITAVQAQ

SEQ ID NO: 201 - protein, 11 aa, synthetic construct
  REGION 1..11: note = Synthetic
AQLLLTPQLAQ

SEQ ID NO: 202 - protein, 11 aa, synthetic construct
  REGION 1..11: note = Synthetic
AQLLRSTLPAQ

SEQ ID NO: 203 - protein, 11 aa, synthetic construct
  REGION 1..11: note = Synthetic
AQLLSATTHAQ

SEQ ID NO: 204 - protein, 11 aa, synthetic construct
  REGION 1..11: note = Synthetic
AQLLSSSNSAQ

SEQ ID NO: 205 - protein, 11 aa, synthetic construct
  REGION 1..11: note = Synthetic
AQLMSILTMAQ

SEQ ID NO: 206 - protein, 11 aa, synthetic construct
  REGION 1..11: note = Synthetic
AQLMSSVSAAQ

SEQ ID NO: 207 - protein, 11 aa, synthetic construct
  REGION 1..11: note = Synthetic
AQLNHSVSGAQ

SEQ ID NO: 208 - protein, 11 aa, synthetic construct
  REGION 1..11: note = Synthetic
AQLNHTSPGAQ

SEQ ID NO: 209 - protein, 11 aa, synthetic construct
  REGION 1..11: note = Synthetic
AQLNPQGTSAQ

SEQ ID NO: 210 - protein, 11 aa, synthetic construct
  REGION 1..11: note = Synthetic
AQLNSDKSSAQ

SEQ ID NO: 211 - protein, 11 aa, synthetic construct
  REGION 1..11: note = Synthetic
AQLNSTRTYAQ

SEQ ID NO: 212 - protein, 11 aa, synthetic construct
  REGION 1..11: note = Synthetic
AQLNTAQSTAQ

SEQ ID NO: 213 - protein, 11 aa, synthetic construct
  REGION 1..11: note = Synthetic
AQLNTGSNAAQ

SEQ ID NO: 214 - protein, 11 aa, synthetic construct
  REGION 1..11: note = Synthetic
AQLNVSTTSAQ

SEQ ID NO: 215 - protein, 11 aa, synthetic construct
  REGION 1..11: note = Synthetic
AQLPASDISAQ

SEQ ID NO: 216 - protein, 11 aa, synthetic construct
  REGION 1..11: note = Synthetic
AQLPRDKWEAQ

SEQ ID NO: 217 - protein, 11 aa, synthetic construct
  REGION 1..11: note = Synthetic
AQLPSETKFAQ

SEQ ID NO: 218 - protein, 11 aa, synthetic construct
  REGION 1..11: note = Synthetic
AQLQSNRSDAQ

SEQ ID NO: 219 - protein, 11 aa, synthetic construct
  REGION 1..11: note = Synthetic
AQLQSRSFGAQ

SEQ ID NO: 220 - protein, 11 aa, synthetic construct
  REGION 1..11: note = Synthetic
AQLQTSLSLAQ

SEQ ID NO: 221 - protein, 11 aa, synthetic construct
  REGION 1..11: note = Synthetic
AQLRNSESTAQ

SEQ ID NO: 222 - protein, 11 aa, synthetic construct
  REGION 1..11: note = Synthetic
AQLRNSSELAQ

SEQ ID NO: 223 - protein, 11 aa, synthetic construct
  REGION 1..11: note = Synthetic
AQLRTDSLEAQ

SEQ ID NO: 224 - protein, 11 aa, synthetic construct
  REGION 1..11: note = Synthetic
AQLSDHNLKAQ

SEQ ID NO: 225 - protein, 11 aa, synthetic construct
  REGION 1..11: note = Synthetic
AQLSGFNVLAQ

SEQ ID NO: 226 - protein, 11 aa, synthetic construct
  REGION 1..11: note = Synthetic
AQLSGHFSFAQ

SEQ ID NO: 227 - protein, 11 aa, synthetic construct
  REGION 1..11: note = Synthetic
AQLSGMSGNAQ

SEQ ID NO: 228 - protein, 11 aa, synthetic construct
  REGION 1..11: note = Synthetic
AQLSGQSMDAQ

SEQ ID NO: 229 - protein, 11 aa, synthetic construct
  REGION 1..11: note = Synthetic
AQLSGQVTMAQ

SEQ ID NO: 230 - protein, 11 aa, synthetic construct
  REGION 1..11: note = Synthetic
AQLSNESRSAQ

SEQ ID NO: 231 - protein, 11 aa, synthetic construct
  REGION 1..11: note = Synthetic
AQLSNITNSAQ

SEQ ID NO: 232 - protein, 11 aa, synthetic construct
  REGION 1..11: note = Synthetic
AQLSQASSAAQ

SEQ ID NO: 233 - protein, 11 aa, synthetic construct
  REGION 1..11: note = Synthetic
AQLSQSGKEAQ

SEQ ID NO: 234 - protein, 11 aa, synthetic construct
  REGION 1..11: note = Synthetic
AQLSRSFVPAQ

SEQ ID NO: 235 - protein, 11 aa, synthetic construct
  REGION 1..11: note = Synthetic
AQLSRSSSPAQ

SEQ ID NO: 236 - protein, 11 aa, synthetic construct
  REGION 1..11: note = Synthetic
AQLSSANLQAQ

SEQ ID NO: 237 - protein, 11 aa, synthetic construct
  REGION 1..11: note = Synthetic
AQLSSSITQAQ

SEQ ID NO: 238 - protein, 11 aa, synthetic construct
  REGION 1..11: note = Synthetic
AQLSSSVVNAQ

SEQ ID NO: 239 - protein, 11 aa, synthetic construct
  REGION 1..11: note = Synthetic
AQLSSTLTSAQ

SEQ ID NO: 240 - protein, 11 aa, synthetic construct
  REGION 1..11: note = Synthetic
AQLSSTYANAQ

SEQ ID NO: 241 - protein, 11 aa, synthetic construct
  REGION 1..11: note = Synthetic
AQLSTDASQAQ

SEQ ID NO: 242 - protein, 11 aa, synthetic construct
  REGION 1..11: note = Synthetic
AQLSTISSHAQ

SEQ ID NO: 243 - protein, 11 aa, synthetic construct
  REGION 1..11: note = Synthetic
AQLSTSGPSAQ

SEQ ID NO: 244 - protein, 11 aa, synthetic construct
  REGION 1..11: note = Synthetic
AQLSVAQGSAQ

SEQ ID NO: 245 - protein, 11 aa, synthetic construct
  REGION 1..11: note = Synthetic
AQLSVLSQIAQ

SEQ ID NO: 246 - protein, 11 aa, synthetic construct
  REGION 1..11: note = Synthetic
AQLTGNQSVAQ

SEQ ID NO: 247 - protein, 11 aa, synthetic construct
  REGION 1..11: note = Synthetic
AQLTHTIPSAQ

SEQ ID NO: 248 - protein, 11 aa, synthetic construct
  REGION 1..11: note = Synthetic
AQLTNVIMGAQ

SEQ ID NO: 249 - protein, 11 aa, synthetic construct
  REGION 1..11: note = Synthetic
AQLTSLPQVAQ

SEQ ID NO: 250 - protein, 11 aa, synthetic construct
  REGION 1..11: note = Synthetic
AQLTTPSAAAQ

SEQ ID NO: 251 - protein, 11 aa, synthetic construct
  REGION 1..11: note = Synthetic
AQLTTSSTLAQ

SEQ ID NO: 252 - protein, 11 aa, synthetic construct
  REGION 1..11: note = Synthetic
AQLTTTHSQAQ

SEQ ID NO: 253 - protein, 11 aa, synthetic construct
  REGION 1..11: note = Synthetic
AQLVIMGMSAQ

SEQ ID NO: 254 - protein, 11 aa, synthetic construct
  REGION 1..11: note = Synthetic
AQLVSSESRAQ

SEQ ID NO: 255 - protein, 11 aa, synthetic construct
  REGION 1..11: note = Synthetic
AQLVTAGTAAQ

SEQ ID NO: 256 - protein, 11 aa, synthetic construct
  REGION 1..11: note = Synthetic
AQMDTHNLVAQ

SEQ ID NO: 257 - protein, 11 aa, synthetic construct
  REGION 1..11: note = Synthetic
AQMDYSTSPAQ

SEQ ID NO: 258 - protein, 11 aa, synthetic construct
  REGION 1..11: note = Synthetic
AQMGSSGQMAQ

SEQ ID NO: 259 - protein, 11 aa, synthetic construct
  REGION 1..11: note = Synthetic
AQMGTAHTIAQ

SEQ ID NO: 260 - protein, 11 aa, synthetic construct
  REGION 1..11: note = Synthetic
AQMGTHVVTAQ

SEQ ID NO: 261 - protein, 11 aa, synthetic construct
  REGION 1..11: note = Synthetic
AQMHDGRLVAQ

SEQ ID NO: 262 - protein, 11 aa, synthetic construct
  REGION 1..11: note = Synthetic
AQMLAKTATAQ

SEQ ID NO: 263 - protein, 11 aa, synthetic construct
  REGION 1..11: note = Synthetic
AQMLSSTMRAQ

SEQ ID NO: 264 - protein, 11 aa, synthetic construct
  REGION 1..11: note = Synthetic
AQMLSTTSGAQ

SEQ ID NO: 265 - protein, 11 aa, synthetic construct
  REGION 1..11: note = Synthetic
AQMLVKHAGAQ

SEQ ID NO: 266 - protein, 11 aa, synthetic construct
  REGION 1..11: note = Synthetic
AQMMSSTNAAQ

SEQ ID NO: 267 - protein, 11 aa, synthetic construct
  REGION 1..11: note = Synthetic
AQMNHNQQHAQ

SEQ ID NO: 268 - protein, 11 aa, synthetic construct
  REGION 1..11: note = Synthetic
AQMNHTSNKAQ

SEQ ID NO: 269 - protein, 11 aa, synthetic construct
  REGION 1..11: note = Synthetic
AQMNNKSPGAQ

SEQ ID NO: 270 - protein, 11 aa, synthetic construct
  REGION 1..11: note = Synthetic
AQMNNSTRPAQ

SEQ ID NO: 271 - protein, 11 aa, synthetic construct
  REGION 1..11: note = Synthetic
AQMNSSSHSAQ

SEQ ID NO: 272 - protein, 11 aa, synthetic construct
  REGION 1..11: note = Synthetic
AQMSGNSMAAQ

SEQ ID NO: 273 - protein, 11 aa, synthetic construct
  REGION 1..11: note = Synthetic
AQMSTSYSSAQ

SEQ ID NO: 274 - protein, 11 aa, synthetic construct
  REGION 1..11: note = Synthetic
AQMYLFNNSAQ

SEQ ID NO: 275 - protein, 11 aa, synthetic construct
  REGION 1..11: note = Synthetic
AQNAGYSAAAQ

SEQ ID NO: 276 - protein, 11 aa, synthetic construct
  REGION 1..11: note = Synthetic
AQNANHAMPAQ

SEQ ID NO: 277 - protein, 11 aa, synthetic construct
  REGION 1..11: note = Synthetic
AQNAQGTHVAQ

SEQ ID NO: 278 - protein, 11 aa, synthetic construct
  REGION 1..11: note = Synthetic
AQNASEKSSAQ

SEQ ID NO: 279 - protein, 11 aa, synthetic construct
  REGION 1..11: note = Synthetic
AQNASNSTTAQ

SEQ ID NO: 280 - protein, 11 aa, synthetic construct
  REGION 1..11: note = Synthetic
AQNGLRADTAQ

SEQ ID NO: 281 - protein, 11 aa, synthetic construct
  REGION 1..11: note = Synthetic
AQNGRTGTGAQ

SEQ ID NO: 282 - protein, 11 aa, synthetic construct
  REGION 1..11: note = Synthetic
AQNGRTTTVAQ

SEQ ID NO: 283 - protein, 11 aa, synthetic construct
  REGION 1..11: note = Synthetic
AQNGSITGTAQ

SEQ ID NO: 284 - protein, 11 aa, synthetic construct
  REGION 1..11: note = Synthetic
AQNGSRGAAAQ

SEQ ID NO: 285 - protein, 11 aa, synthetic construct
  REGION 1..11: note = Synthetic
AQNHEQYQWAQ

SEQ ID NO: 286 - protein, 11 aa, synthetic construct
  REGION 1..11: note = Synthetic
AQNHSGTTSAQ

SEQ ID NO: 287 - protein, 11 aa, synthetic construct
  REGION 1..11: note = Synthetic
AQNHTGVSPAQ

SEQ ID NO: 288 - protein, 11 aa, synthetic construct
  REGION 1..11: note = Synthetic
AQNHVGGATAQ

SEQ ID NO: 289 - protein, 11 aa, synthetic construct
  REGION 1..11: note = Synthetic
AQNIQSLSTAQ

SEQ ID NO: 290 - protein, 11 aa, synthetic construct
  REGION 1..11: note = Synthetic
AQNLGLISVAQ

SEQ ID NO: 291 - protein, 11 aa, synthetic construct
  REGION 1..11: note = Synthetic
AQNLSLTHEAQ

SEQ ID NO: 292 - protein, 11 aa, synthetic construct
  REGION 1..11: note = Synthetic
AQNMSSNKDAQ

SEQ ID NO: 293 - protein, 11 aa, synthetic construct
  REGION 1..11: note = Synthetic
AQNMTSMQSAQ

SEQ ID NO: 294 - protein, 11 aa, synthetic construct
  REGION 1..11: note = Synthetic
AQNPTRSLSAQ

SEQ ID NO: 295 - protein, 11 aa, synthetic construct
  REGION 1..11: note = Synthetic
AQNQCQSHVAQ

SEQ ID NO: 296 - protein, 11 aa, synthetic construct
  REGION 1..11: note = Synthetic
AQNQFTATEAQ

SEQ ID NO: 297 - protein, 11 aa, synthetic construct
  REGION 1..11: note = Synthetic
AQNQLGNSAAQ

SEQ ID NO: 298 - protein, 11 aa, synthetic construct
  REGION 1..11: note = Synthetic
AQNQQNKANAQ

SEQ ID NO: 299 - protein, 11 aa, synthetic construct
  REGION 1..11: note = Synthetic
AQNRNEVHSAQ

SEQ ID NO: 300 - protein, 11 aa, synthetic construct
  REGION 1..11: note = Synthetic
AQNRSDSAMAQ

SEQ ID NO: 301 - protein, 11 aa, synthetic construct
  REGION 1..11: note = Synthetic
AQNSAFTAQAQ

SEQ ID NO: 302 - protein, 11 aa, synthetic construct
  REGION 1..11: note = Synthetic
AQNSFSPATAQ

SEQ ID NO: 303 - protein, 11 aa, synthetic construct
  REGION 1..11: note = Synthetic
AQNSGNYQIAQ

SEQ ID NO: 304 - protein, 11 aa, synthetic construct
  REGION 1..11: note = Synthetic
AQNSHGHALAQ

SEQ ID NO: 305 - protein, 11 aa, synthetic construct
  REGION 1..11: note = Synthetic
AQNSPGYTAAQ

SEQ ID NO: 306 - protein, 11 aa, synthetic construct
  REGION 1..11: note = Synthetic
AQNSSAATGAQ

SEQ ID NO: 307 - protein, 11 aa, synthetic construct
  REGION 1..11: note = Synthetic
AQNSSGHGVAQ

SEQ ID NO: 308 - protein, 11 aa, synthetic construct
  REGION 1..11: note = Synthetic
AQNSSSGSAAQ

SEQ ID NO: 309 - protein, 11 aa, synthetic construct
  REGION 1..11: note = Synthetic
AQNSSSSPGAQ

SEQ ID NO: 310 - protein, 11 aa, synthetic construct
  REGION 1..11: note = Synthetic
AQNSTSISHAQ

SEQ ID NO: 311 - protein, 11 aa, synthetic construct
  REGION 1..11: note = Synthetic
AQNTLSNISAQ

SEQ ID NO: 312 - protein, 11 aa, synthetic construct
  REGION 1..11: note = Synthetic
AQNTQTHSSAQ

SEQ ID NO: 313 - protein, 11 aa, synthetic construct
  REGION 1..11: note = Synthetic
AQNTTQPSNAQ

SEQ ID NO: 314 - protein, 11 aa, synthetic construct
  REGION 1..11: note = Synthetic
AQNVMSSGAAQ

SEQ ID NO: 315 - protein, 11 aa, synthetic construct
  REGION 1..11: note = Synthetic
AQNVQSSNLAQ

SEQ ID NO: 316 - protein, 11 aa, synthetic construct
  REGION 1..11: note = Synthetic
AQNYCGKECAQ

SEQ ID NO: 317 - protein, 11 aa, synthetic construct
  REGION 1..11: note = Synthetic
AQPATGTSGAQ

SEQ ID NO: 318 - protein, 11 aa, synthetic construct
  REGION 1..11: note = Synthetic
AQPAVGSAGAQ

SEQ ID NO: 319 - protein, 11 aa, synthetic construct
  REGION 1..11: note = Synthetic
AQPIGGSNMAQ

SEQ ID NO: 320 - protein, 11 aa, synthetic construct
  REGION 1..11: note = Synthetic
AQPIHQTGVAQ

SEQ ID NO: 321 - protein, 11 aa, synthetic construct
  REGION 1..11: note = Synthetic
AQPLQTSNAAQ

SEQ ID NO: 322 - protein, 11 aa, synthetic construct
  REGION 1..11: note = Synthetic
AQPLTGQTNAQ

SEQ ID NO: 323 - protein, 11 aa, synthetic construct
  REGION 1..11: note = Synthetic
AQPLYNQTPAQ

SEQ ID NO: 324 - protein, 11 aa, synthetic construct
  REGION 1..11: note = Synthetic
AQPPKPPSIAQ

SEQ ID NO: 325 - protein, 11 aa, synthetic construct
  REGION 1..11: note = Synthetic
AQPQPHRLLAQ

SEQ ID NO: 326 - protein, 11 aa, synthetic construct
  REGION 1..11: note = Synthetic
AQPQRSQHIAQ

SEQ ID NO: 327 - protein, 11 aa, synthetic construct
  REGION 1..11: note = Synthetic
AQPQSNVSTAQ

SEQ ID NO: 328 - protein, 11 aa, synthetic construct
  REGION 1..11: note = Synthetic
AQPSAEQAQAQ

SEQ ID NO: 329 - protein, 11 aa, synthetic construct
  REGION 1..11: note = Synthetic
AQPSGAYGEAQ

SEQ ID NO: 330 - protein, 11 aa, synthetic construct
  REGION 1..11: note = Synthetic
AQPSGQKIDAQ

SEQ ID NO: 331 - protein, 11 aa, synthetic construct
  REGION 1..11: note = Synthetic
AQPSGSHSIAQ

SEQ ID NO: 332 - protein, 11 aa, synthetic construct
  REGION 1..11: note = Synthetic
AQPSGTNIGAQ

SEQ ID NO: 333 - protein, 11 aa, synthetic construct
  REGION 1..11: note = Synthetic
AQPSNALAGAQ

SEQ ID NO: 334 - protein, 11 aa, synthetic construct
  REGION 1..11: note = Synthetic
AQPSNPQSYAQ

SEQ ID NO: 335 - protein, 11 aa, synthetic construct
  REGION 1..11: note = Synthetic
AQPSPSTMRAQ

SEQ ID NO: 336 - protein, 11 aa, synthetic construct
  REGION 1..11: note = Synthetic
AQPSRDVTPAQ

SEQ ID NO: 337 - protein, 11 aa, synthetic construct
  REGION 1..11: note = Synthetic
AQPSSYSDSAQ

SEQ ID NO: 338 - protein, 11 aa, synthetic construct
  REGION 1..11: note = Synthetic
AQPVAAHVRAQ

SEQ ID NO: 339 - protein, 11 aa, synthetic construct
  REGION 1..11: note = Synthetic
AQPVHGSTDAQ

SEQ ID NO: 340 - protein, 11 aa, synthetic construct
  REGION 1..11: note = Synthetic
AQQAAVSGGAQ

SEQ ID NO: 341 - protein, 11 aa, synthetic construct
  REGION 1..11: note = Synthetic
AQQATHVALAQ

SEQ ID NO: 342 - protein, 11 aa, synthetic construct
  REGION 1..11: note = Synthetic
AQQDSTKALAQ

SEQ ID NO: 343 - protein, 11 aa, synthetic construct
  REGION 1..11: note = Synthetic
AQQIHGPMQAQ

SEQ ID NO: 344 - protein, 11 aa, synthetic construct
  REGION 1..11: note = Synthetic
AQQLLVSVTAQ

SEQ ID NO: 345 - protein, 11 aa, synthetic construct
  REGION 1..11: note = Synthetic
AQQLSGSLMAQ

SEQ ID NO: 346 - protein, 11 aa, synthetic construct
  REGION 1..11: note = Synthetic
AQQLSHEKSAQ

SEQ ID NO: 347 - protein, 11 aa, synthetic construct
  REGION 1..11: note = Synthetic
AQQNNTHGGAQ

SEQ ID NO: 348 - protein, 11 aa, synthetic construct
  REGION 1..11: note = Synthetic
AQQNSGPLHAQ

SEQ ID NO: 349 - protein, 11 aa, synthetic construct
  REGION 1..11: note = Synthetic
AQQRTDQQTAQ

SEQ ID NO: 350 - protein, 11 aa, synthetic construct
  REGION 1..11: note = Synthetic
AQQVAHTQIAQ

SEQ ID NO: 351 - protein, 11 aa, synthetic construct
  REGION 1..11: note = Synthetic
AQQVGKSSAAQ

SEQ ID NO: 352 - protein, 11 aa, synthetic construct
  REGION 1..11: note = Synthetic
AQQVISTGSAQ

SEQ ID NO: 353 - protein, 11 aa, synthetic construct
  REGION 1..11: note = Synthetic
AQQVRTNTSAQ

SEQ ID NO: 354 - protein, 11 aa, synthetic construct
  REGION 1..11: note = Synthetic
AQRADRVQEAQ

SEQ ID NO: 355 - protein, 11 aa, synthetic construct
  REGION 1..11: note = Synthetic
AQRDKMVFMAQ

SEQ ID NO: 356 - protein, 11 aa, synthetic construct
  REGION 1..11: note = Synthetic
AQRDSVTDSAQ

SEQ ID NO: 357 - protein, 11 aa, synthetic construct
  REGION 1..11: note = Synthetic
AQRESRVSQAQ

SEQ ID NO: 358 - protein, 11 aa, synthetic construct
  REGION 1..11: note = Synthetic
AQRGHGQEIAQ

SEQ ID NO: 359 - protein, 11 aa, synthetic construct
  REGION 1..11: note = Synthetic
AQRGLSAVNAQ

SEQ ID NO: 360 - protein, 11 aa, synthetic construct
  REGION 1..11: note = Synthetic
AQRIGSSAIAQ

SEQ ID NO: 361 - protein, 11 aa, synthetic construct
  REGION 1..11: note = Synthetic
AQRISSETSAQ

SEQ ID NO: 362 - protein, 11 aa, synthetic construct
  REGION 1..11: note = Synthetic
AQRLDIHMCAQ

SEQ ID NO: 363 - protein, 11 aa, synthetic construct
  REGION 1..11: note = Synthetic
AQRNTSNQEAQ

SEQ ID NO: 364 - protein, 11 aa, synthetic construct
  REGION 1..11: note = Synthetic
AQRSNNCIGAQ

SEQ ID NO: 365 - protein, 11 aa, synthetic construct
  REGION 1..11: note = Synthetic
AQRSNVDGLAQ

SEQ ID NO: 366 - protein, 11 aa, synthetic construct
  REGION 1..11: note = Synthetic
AQRSSLHTSAQ

SEQ ID NO: 367 - protein, 11 aa, synthetic construct
  REGION 1..11: note = Synthetic
AQRSTESLHAQ

SEQ ID NO: 368 - protein, 11 aa, synthetic construct
  REGION 1..11: note = Synthetic
AQRTAANHEAQ

SEQ ID NO: 369 - protein, 11 aa, synthetic construct
  REGION 1..11: note = Synthetic
AQRTDGTSIAQ

SEQ ID NO: 370 - protein, 11 aa, synthetic construct
  REGION 1..11: note = Synthetic
AQRTHSAMPAQ

SEQ ID NO: 371 - protein, 11 aa, synthetic construct
  REGION 1..11: note = Synthetic
AQRTSGPAAAQ

SEQ ID NO: 372 - protein, 11 aa, synthetic construct
  REGION 1..11: note = Synthetic
AQSAPNRTDAQ

SEQ ID NO: 373 - protein, 11 aa, synthetic construct
  REGION 1..11: note = Synthetic
AQSAQSAIQAQ

SEQ ID NO: 374 - protein, 11 aa, synthetic construct
  REGION 1..11: note = Synthetic
AQSARGMSGAQ

SEQ ID NO: 375 - protein, 11 aa, synthetic construct
  REGION 1..11: note = Synthetic
AQSASRDPAAQ

SEQ ID NO: 376 - protein, 11 aa, synthetic construct
  REGION 1..11: note = Synthetic
AQSDHSVRDAQ

SEQ ID NO: 377 - protein, 11 aa, synthetic construct
  REGION 1..11: note = Synthetic
AQSDKNLQQAQ

SEQ ID NO: 378 - protein, 11 aa, synthetic construct
  REGION 1..11: note = Synthetic
AQSDLICCPAQ

SEQ ID NO: 379 - protein, 11 aa, synthetic construct
  REGION 1..11: note = Synthetic
AQSERSYPSAQ

SEQ ID NO: 380 - protein, 11 aa, synthetic construct
  REGION 1..11: note = Synthetic
AQSFSGQSLAQ

SEQ ID NO: 381 - protein, 11 aa, synthetic construct
  REGION 1..11: note = Synthetic
AQSFSNVSPAQ

SEQ ID NO: 382 - protein, 11 aa, synthetic construct
  REGION 1..11: note = Synthetic
AQSGLNTLAAQ

SEQ ID NO: 383 - protein, 11 aa, synthetic construct
  REGION 1..11: note = Synthetic
AQSGNHNPAAQ

SEQ ID NO: 384 - protein, 11 aa, synthetic construct
  REGION 1..11: note = Synthetic
AQSGPVQNQAQ

SEQ ID NO: 385 - protein, 11 aa, synthetic construct
  REGION 1..11: note = Synthetic
AQSGSSHERAQ

SEQ ID NO: 386 - protein, 11 aa, synthetic construct
  REGION 1..11: note = Synthetic
AQSGSVMSSAQ

SEQ ID NO: 387 - protein, 11 aa, synthetic construct
  REGION 1..11: note = Synthetic
AQSHIVSSSAQ

SEQ ID NO: 388 - protein, 11 aa, synthetic construct
  REGION 1..11: note = Synthetic
AQSHLSSQVAQ

SEQ ID NO: 389 - protein, 11 aa, synthetic construct
  REGION 1..11: note = Synthetic
AQSHNSAFEAQ

SEQ ID NO: 390 - protein, 11 aa, synthetic construct
  REGION 1..11: note = Synthetic
AQSIARDLAAQ

SEQ ID NO: 391 - protein, 11 aa, synthetic construct
  REGION 1..11: note = Synthetic
AQSIGSSYPAQ

SEQ ID NO: 392 - protein, 11 aa, synthetic construct
  REGION 1..11: note = Synthetic
AQSIKQNEGAQ

SEQ ID NO: 393 - protein, 11 aa, synthetic construct
  REGION 1..11: note = Synthetic
AQSITQGSAAQ

SEQ ID NO: 394 - protein, 11 aa, synthetic construct
  REGION 1..11: note = Synthetic
AQSKNDTSMAQ

SEQ ID NO: 395 - protein, 11 aa, synthetic construct
  REGION 1..11: note = Synthetic
AQSLAGTLSAQ

SEQ ID NO: 396 - protein, 11 aa, synthetic construct
  REGION 1..11: note = Synthetic
AQSLANSQTAQ

SEQ ID NO: 397 - protein, 11 aa, synthetic construct
  REGION 1..11: note = Synthetic
AQSLGAQLFAQ

SEQ ID NO: 398 - protein, 11 aa, synthetic construct
  REGION 1..11: note = Synthetic
AQSLHLSSSAQ

SEQ ID NO: 399 - protein, 11 aa, synthetic construct
  REGION 1..11: note = Synthetic
AQSLSHTRPAQ

SEQ ID NO: 400 - protein, 11 aa, synthetic construct
  REGION 1..11: note = Synthetic
AQSLSQTVGAQ

SEQ ID NO: 401 - protein, 11 aa, synthetic construct
  REGION 1..11: note = Synthetic
AQSLSSTGGAQ

SEQ ID NO: 402 - protein, 11 aa, synthetic construct
  REGION 1..11: note = Synthetic
AQSLVSLKEAQ

SEQ ID NO: 403 - protein, 11 aa, synthetic construct
  REGION 1..11: note = Synthetic
AQSLVVTTPAQ

SEQ ID NO: 404 - protein, 11 aa, synthetic construct
  REGION 1..11: note = Synthetic
AQSMGSSNWAQ

SEQ ID NO: 405 - protein, 11 aa, synthetic construct
  REGION 1..11: note = Synthetic
AQSMISSNRAQ

SEQ ID NO: 406 - protein, 11 aa, synthetic construct
  REGION 1..11: note = Synthetic
AQSMMSLDQAQ

SEQ ID NO: 407 - protein, 11 aa, synthetic construct
  REGION 1..11: note = Synthetic
AQSMPQPLVAQ

SEQ ID NO: 408 - protein, 11 aa, synthetic construct
  REGION 1..11: note = Synthetic
AQSMSSNLTAQ

SEQ ID NO: 409 - protein, 11 aa, synthetic construct
  REGION 1..11: note = Synthetic
AQSMTLSNYAQ

SEQ ID NO: 410 - protein, 11 aa, synthetic construct
  REGION 1..11: note = Synthetic
AQSNATHRFAQ

SEQ ID NO: 411 - protein, 11 aa, synthetic construct
  REGION 1..11: note = Synthetic
AQSNHTKPIAQ

SEQ ID NO: 412 - protein, 11 aa, synthetic construct
  REGION 1..11: note = Synthetic
AQSNQTRHVAQ

SEQ ID NO: 413 - protein, 11 aa, synthetic construct
  REGION 1..11: note = Synthetic
AQSPKPPSTAQ

SEQ ID NO: 414 - protein, 11 aa, synthetic construct
  REGION 1..11: note = Synthetic
AQSQDTASKAQ

SEQ ID NO: 415 - protein, 11 aa, synthetic construct
  REGION 1..11: note = Synthetic
AQSQIAEPAAQ

SEQ ID NO: 416 - protein, 11 aa, synthetic construct
  REGION 1..11: note = Synthetic
AQSQKAPDIAQ

SEQ ID NO: 417 - protein, 11 aa, synthetic construct
  REGION 1..11: note = Synthetic
AQSQLSPSNAQ

SEQ ID NO: 418 - protein, 11 aa, synthetic construct
  REGION 1..11: note = Synthetic
AQSQMSSAGAQ

SEQ ID NO: 419 - protein, 11 aa, synthetic construct
  REGION 1..11: note = Synthetic
AQSQSSSQTAQ

SEQ ID NO: 420 - protein, 11 aa, synthetic construct
  REGION 1..11: note = Synthetic
AQSRESPSTAQ

SEQ ID NO: 421 - protein, 11 aa, synthetic construct
  REGION 1..11: note = Synthetic
AQSRLLESSAQ

SEQ ID NO: 422 - protein, 11 aa, synthetic construct
  REGION 1..11: note = Synthetic
AQSRNQPAEAQ

SEQ ID NO: 423 - protein, 11 aa, synthetic construct
  REGION 1..11: note = Synthetic
AQSRSSDAFAQ

SEQ ID NO: 424 - protein, 11 aa, synthetic construct
  REGION 1..11: note = Synthetic
AQSRTDLLSAQ

SEQ ID NO: 425 - protein, 11 aa, synthetic construct
  REGION 1..11: note = Synthetic
AQSRTSNQAAQ

SEQ ID NO: 426 - protein, 11 aa, synthetic construct
  REGION 1..11: note = Synthetic
AQSRVEEMNAQ

SEQ ID NO: 427 - protein, 11 aa, synthetic construct
  REGION 1..11: note = Synthetic
AQSRVNTLDAQ

SEQ ID NO: 428 - protein, 11 aa, synthetic construct
  REGION 1..11: note = Synthetic
AQSSAVLLNAQ

SEQ ID NO: 429 - protein, 11 aa, synthetic construct
  REGION 1..11: note = Synthetic
AQSSDRVSSAQ

SEQ ID NO: 430 - protein, 11 aa, synthetic construct
  REGION 1..11: note = Synthetic
AQSSGHSSEAQ

SEQ ID NO: 431 - protein, 11 aa, synthetic construct
  REGION 1..11: note = Synthetic
AQSSGLRLGAQ

SEQ ID NO: 432 - protein, 11 aa, synthetic construct
  REGION 1..11: note = Synthetic
AQSSGSREVAQ

SEQ ID NO: 433 - protein, 11 aa, synthetic construct
  REGION 1..11: note = Synthetic
AQSSHDTLQAQ

SEQ ID NO: 434 - protein, 11 aa, synthetic construct
  REGION 1..11: note = Synthetic
AQSSLSVLWAQ

SEQ ID NO: 435 - protein, 11 aa, synthetic construct
  REGION 1..11: note = Synthetic
AQSSLSVVPAQ

SEQ ID NO: 436 - protein, 11 aa, synthetic construct
  REGION 1..11: note = Synthetic
AQSSMWHKDAQ

SEQ ID NO: 437 - protein, 11 aa, synthetic construct
  REGION 1..11: note = Synthetic
AQSSQSFGYAQ

SEQ ID NO: 438 - protein, 11 aa, synthetic construct
  REGION 1..11: note = Synthetic
AQSSREVVTAQ

SEQ ID NO: 439 - protein, 11 aa, synthetic construct
  REGION 1..11: note = Synthetic
AQSSRGETPAQ

SEQ ID NO: 440 - protein, 11 aa, synthetic construct
  REGION 1..11: note = Synthetic
AQSSSQSSDAQ

SEQ ID NO: 441 - protein, 11 aa, synthetic construct
  REGION 1..11: note = Synthetic
AQSSTQHEKAQ

SEQ ID NO: 442 - protein, 11 aa, synthetic construct
  REGION 1..11: note = Synthetic
AQSSVSRTSAQ

SEQ ID NO: 443 - protein, 11 aa, synthetic construct
  REGION 1..11: note = Synthetic
AQSSYGYRSAQ

SEQ ID NO: 444 - protein, 11 aa, synthetic construct
  REGION 1..11: note = Synthetic
AQSTALTTGAQ

SEQ ID NO: 445 - protein, 11 aa, synthetic construct
  REGION 1..11: note = Synthetic
AQSTATSPAAQ

SEQ ID NO: 446 - protein, 11 aa, synthetic construct
  REGION 1..11: note = Synthetic
AQSTGQGFTAQ

SEQ ID NO: 447 - protein, 11 aa, synthetic construct
  REGION 1..11: note = Synthetic
AQSTGQGINAQ

SEQ ID NO: 448 - protein, 11 aa, synthetic construct
  REGION 1..11: note = Synthetic
AQSTGTSGQAQ

SEQ ID NO: 449 - protein, 11 aa, synthetic construct
  REGION 1..11: note = Synthetic
AQSTISTGSAQ

SEQ ID NO: 450 - protein, 11 aa, synthetic construct
  REGION 1..11: note = Synthetic
AQSTKQNVNAQ

SEQ ID NO: 451 - protein, 11 aa, synthetic construct
  REGION 1..11: note = Synthetic
AQSTMAIGSAQ

SEQ ID NO: 452 - protein, 11 aa, synthetic construct
  REGION 1..11: note = Synthetic
AQSTMGALKAQ

SEQ ID NO: 453 - protein, 11 aa, synthetic construct
  REGION 1..11: note = Synthetic
AQSTNRAEQAQ

SEQ ID NO: 454 - protein, 11 aa, synthetic construct
  REGION 1..11: note = Synthetic
AQSTSALGPAQ

SEQ ID NO: 455 - protein, 11 aa, synthetic construct
  REGION 1..11: note = Synthetic
AQSTSDYSRAQ

SEQ ID NO: 456 - protein, 11 aa, synthetic construct
  REGION 1..11: note = Synthetic
AQSTSPTYVAQ

SEQ ID NO: 457 - protein, 11 aa, synthetic construct
  REGION 1..11: note = Synthetic
AQSTSSITVAQ

SEQ ID NO: 458 - protein, 11 aa, synthetic construct
  REGION 1..11: note = Synthetic
AQSTSSPTSAQ

SEQ ID NO: 459 - protein, 11 aa, synthetic construct
  REGION 1..11: note = Synthetic
AQSTSYASIAQ

SEQ ID NO: 460 - protein, 11 aa, synthetic construct
  REGION 1..11: note = Synthetic
AQSTTRVVDAQ

SEQ ID NO: 461 - protein, 11 aa, synthetic construct
  REGION 1..11: note = Synthetic
AQSVAPIANAQ

SEQ ID NO: 462 - protein, 11 aa, synthetic construct
  REGION 1..11: note = Synthetic
AQSVGNGNIAQ

SEQ ID NO: 463 - protein, 11 aa, synthetic construct
  REGION 1..11: note = Synthetic
AQSVRSGFKAQ

SEQ ID NO: 464 - protein, 11 aa, synthetic construct
  REGION 1..11: note = Synthetic
AQSVSDGGFAQ

SEQ ID NO: 465 - protein, 11 aa, synthetic construct
  REGION 1..11: note = Synthetic
AQSVSSNIVAQ

SEQ ID NO: 466 - protein, 11 aa, synthetic construct
  REGION 1..11: note = Synthetic
AQSVSTAAQAQ

SEQ ID NO: 467 - protein, 11 aa, synthetic construct
  REGION 1..11: note = Synthetic
AQTAHNVEQAQ

SEQ ID NO: 468 - protein, 11 aa, synthetic construct
  REGION 1..11: note = Synthetic
AQTAPQSMDAQ

SEQ ID NO: 469 - protein, 11 aa, synthetic construct
  REGION 1..11: note = Synthetic
AQTDRGQVQAQ

SEQ ID NO: 470 - protein, 11 aa, synthetic construct
  REGION 1..11: note = Synthetic
AQTDSSRESAQ

SEQ ID NO: 471 - protein, 11 aa, synthetic construct
  REGION 1..11: note = Synthetic
AQTEAGSAAAQ

SEQ ID NO: 472 - protein, 11 aa, synthetic construct
  REGION 1..11: note = Synthetic
AQTEQRHAPAQ

SEQ ID NO: 473 - protein, 11 aa, synthetic construct
  REGION 1..11: note = Synthetic
AQTFGKASDAQ

SEQ ID NO: 474 - protein, 11 aa, synthetic construct
  REGION 1..11: note = Synthetic
AQTGAASNIAQ

SEQ ID NO: 475 - protein, 11 aa, synthetic construct
  REGION 1..11: note = Synthetic
AQTGLSAEGAQ

SEQ ID NO: 476 - protein, 11 aa, synthetic construct
  REGION 1..11: note = Synthetic
AQTGTVHSTAQ

SEQ ID NO: 477 - protein, 11 aa, synthetic construct
  REGION 1..11: note = Synthetic
AQTHMQVGAAQ

SEQ ID NO: 478 - protein, 11 aa, synthetic construct
  REGION 1..11: note = Synthetic
AQTHQPAEKAQ

SEQ ID NO: 479 - protein, 11 aa, synthetic construct
  REGION 1..11: note = Synthetic
AQTHTALPDAQ

SEQ ID NO: 480 - protein, 11 aa, synthetic construct
  REGION 1..11: note = Synthetic
AQTHTRSMIAQ

SEQ ID NO: 481 - protein, 11 aa, synthetic construct
  REGION 1..11: note = Synthetic
AQTINNVVAAQ

SEQ ID NO: 482 - protein, 11 aa, synthetic construct
  REGION 1..11: note = Synthetic
AQTKHSTTEAQ

SEQ ID NO: 483 - protein, 11 aa, synthetic construct
  REGION 1..11: note = Synthetic
AQTLAGSSIAQ

SEQ ID NO: 484 - protein, 11 aa, synthetic construct
  REGION 1..11: note = Synthetic
AQTLRTSDLAQ

SEQ ID NO: 485 - protein, 11 aa, synthetic construct
  REGION 1..11: note = Synthetic
AQTLSGLVMAQ

SEQ ID NO: 486 - protein, 11 aa, synthetic construct
  REGION 1..11: note = Synthetic
AQTLSTHVVAQ

SEQ ID NO: 487 - protein, 11 aa, synthetic construct
  REGION 1..11: note = Synthetic
AQTNITRAQAQ

SEQ ID NO: 488 - protein, 11 aa, synthetic construct
  REGION 1..11: note = Synthetic
AQTNSTRNLAQ

SEQ ID NO: 489 - protein, 11 aa, synthetic construct
  REGION 1..11: note = Synthetic
AQTNSTRNVAQ

SEQ ID NO: 490 - protein, 11 aa, synthetic construct
  REGION 1..11: note = Synthetic
AQTNTHSVTAQ

SEQ ID NO: 491 - protein, 11 aa, synthetic construct
  REGION 1..11: note = Synthetic
AQTNTLHTAAQ

SEQ ID NO: 492 - protein, 11 aa, synthetic construct
  REGION 1..11: note = Synthetic
AQTNTRTMQAQ

SEQ ID NO: 493 - protein, 11 aa, synthetic construct
  REGION 1..11: note = Synthetic
AQTNVSSEIAQ

SEQ ID NO: 494 - protein, 11 aa, synthetic construct
  REGION 1..11: note = Synthetic
AQTPTSSSSAQ

SEQ ID NO: 495 - protein, 11 aa, synthetic construct
  REGION 1..11: note = Synthetic
AQTQASASGAQ

SEQ ID NO: 496 - protein, 11 aa, synthetic construct
  REGION 1..11: note = Synthetic
AQTQGDRGTAQ

SEQ ID NO: 497 - protein, 11 aa, synthetic construct
  REGION 1..11: note = Synthetic
AQTQSKSESAQ

SEQ ID NO: 498 - protein, 11 aa, synthetic construct
  REGION 1..11: note = Synthetic
AQTQTHVTSAQ

SEQ ID NO: 499 - protein, 11 aa, synthetic construct
  REGION 1..11: note = Synthetic
AQTQTVPTNAQ

SEQ ID NO: 500 - protein, 11 aa, synthetic construct
  REGION 1..11: note = Synthetic
AQTRTDSNGAQ

SEQ ID NO: 501 - protein, 11 aa, synthetic construct
  REGION 1..11: note = Synthetic
AQTSAHANQAQ

SEQ ID NO: 502 - protein, 11 aa, synthetic construct
  REGION 1..11: note = Synthetic
AQTSGHSSTAQ

SEQ ID NO: 503 - protein, 11 aa, synthetic construct
  REGION 1..11: note = Synthetic
AQTSLKIVPAQ

SEQ ID NO: 504 - protein, 11 aa, synthetic construct
  REGION 1..11: note = Synthetic
AQTSLTVNIAQ

SEQ ID NO: 505 - protein, 11 aa, synthetic construct
  REGION 1..11: note = Synthetic
AQTSSHSKDAQ

SEQ ID NO: 506 - protein, 11 aa, synthetic construct
  REGION 1..11: note = Synthetic
AQTSSNYVSAQ

SEQ ID NO: 507 - protein, 11 aa, synthetic construct
  REGION 1..11: note = Synthetic
AQTSSTGVQAQ

SEQ ID NO: 508 - protein, 11 aa, synthetic construct
  REGION 1..11: note = Synthetic
AQTSTHTRDAQ

SEQ ID NO: 509 - protein, 11 aa, synthetic construct
  REGION 1..11: note = Synthetic
AQTSVAGSNAQ

SEQ ID NO: 510 - protein, 11 aa, synthetic construct
  REGION 1..11: note = Synthetic
AQTSVTYQDAQ

SEQ ID NO: 511 - protein, 11 aa, synthetic construct
  REGION 1..11: note = Synthetic
AQTTGQITVAQ

SEQ ID NO: 512 - protein, 11 aa, synthetic construct
  REGION 1..11: note = Synthetic
AQTTSQALYAQ

SEQ ID NO: 513 - protein, 11 aa, synthetic construct
  REGION 1..11: note = Synthetic
AQTTSSSVNAQ

SEQ ID NO: 514 - protein, 11 aa, synthetic construct
  REGION 1..11: note = Synthetic
AQTTSTMSTAQ

SEQ ID NO: 515 - protein, 11 aa, synthetic construct
  REGION 1..11: note = Synthetic
AQTTTSPAIAQ

SEQ ID NO: 516 - protein, 11 aa, synthetic construct
  REGION 1..11: note = Synthetic
AQTVGRAMEAQ

SEQ ID NO: 517 - protein, 11 aa, synthetic construct
  REGION 1..11: note = Synthetic
AQTVGVLAAAQ

SEQ ID NO: 518 - protein, 11 aa, synthetic construct
  REGION 1..11: note = Synthetic
AQTVRASTDAQ

SEQ ID NO: 519 - protein, 11 aa, synthetic construct
  REGION 1..11: note = Synthetic
AQTVVSGTGAQ

SEQ ID NO: 520 - protein, 11 aa, synthetic construct
  REGION 1..11: note = Synthetic
AQVANDSFKAQ

SEQ ID NO: 521 - protein, 11 aa, synthetic construct
  REGION 1..11: note = Synthetic
AQVARGDSDAQ

SEQ ID NO: 522 - protein, 11 aa, synthetic construct
  REGION 1..11: note = Synthetic
AQVASGVATAQ

SEQ ID NO: 523 - protein, 11 aa, synthetic construct
  REGION 1..11: note = Synthetic
AQVASRDQFAQ

SEQ ID NO: 524 - protein, 11 aa, synthetic construct
  REGION 1..11: note = Synthetic
AQVATRPDAAQ

SEQ ID NO: 525 - protein, 11 aa, synthetic construct
  REGION 1..11: note = Synthetic
AQVCHLRTSAQ

SEQ ID NO: 526 - protein, 11 aa, synthetic construct
  REGION 1..11: note = Synthetic
AQVDNVTRSAQ

SEQ ID NO: 527 - protein, 11 aa, synthetic construct
  REGION 1..11: note = Synthetic
AQVDSRSTPAQ

SEQ ID NO: 528 - protein, 11 aa, synthetic construct
  REGION 1..11: note = Synthetic
AQVEPIRICAQ

SEQ ID NO: 529 - protein, 11 aa, synthetic construct
  REGION 1..11: note = Synthetic
AQVERLQSGAQ

SEQ ID NO: 530 - protein, 11 aa, synthetic construct
  REGION 1..11: note = Synthetic
AQVFGAHSFAQ

SEQ ID NO: 531 - protein, 11 aa, synthetic construct
  REGION 1..11: note = Synthetic
AQVGASSSTAQ

SEQ ID NO: 532 - protein, 11 aa, synthetic construct
  REGION 1..11: note = Synthetic
AQVGGANLAAQ

SEQ ID NO: 533 - protein, 11 aa, synthetic construct
  REGION 1..11: note = Synthetic
AQVGNAPPHAQ

SEQ ID NO: 534 - protein, 11 aa, synthetic construct
  REGION 1..11: note = Synthetic
AQVGSNVGQAQ

SEQ ID NO: 535 - protein, 11 aa, synthetic construct
  REGION 1..11: note = Synthetic
AQVGSSSVSAQ

SEQ ID NO: 536 - protein, 11 aa, synthetic construct
  REGION 1..11: note = Synthetic
AQVGTVSNSAQ

SEQ ID NO: 537 - protein, 11 aa, synthetic construct
  REGION 1..11: note = Synthetic
AQVGVNRTNAQ

SEQ ID NO: 538 - protein, 11 aa, synthetic construct
  REGION 1..11: note = Synthetic
AQVHLQAHNAQ

SEQ ID NO: 539 - protein, 11 aa, synthetic construct
  REGION 1..11: note = Synthetic
AQVHSLPSVAQ

SEQ ID NO: 540 - protein, 11 aa, synthetic construct
  REGION 1..11: note = Synthetic
AQVHTKYTDAQ

SEQ ID NO: 541 - protein, 11 aa, synthetic construct
  REGION 1..11: note = Synthetic
AQVIAATAEAQ

SEQ ID NO: 542 - protein, 11 aa, synthetic construct
  REGION 1..11: note = Synthetic
AQVIWQNQVAQ

SEQ ID NO: 543 - protein, 11 aa, synthetic construct
  REGION 1..11: note = Synthetic
AQVKSGSMAAQ

SEQ ID NO: 544 - protein, 11 aa, synthetic construct
  REGION 1..11: note = Synthetic
AQVLSGQSPAQ

SEQ ID NO: 545 - protein, 11 aa, synthetic construct
  REGION 1..11: note = Synthetic
AQVLSSMSLAQ

SEQ ID NO: 546 - protein, 11 aa, synthetic construct
  REGION 1..11: note = Synthetic
AQVLTSSDLAQ

SEQ ID NO: 547 - protein, 11 aa, synthetic construct
  REGION 1..11: note = Synthetic
AQVNHGAITAQ

SEQ ID NO: 548 - protein, 11 aa, synthetic construct
  REGION 1..11: note = Synthetic
AQVNSHLTGAQ

SEQ ID NO: 549 - protein, 11 aa, synthetic construct
  REGION 1..11: note = Synthetic
AQVNTHTSSAQ

SEQ ID NO: 550 - protein, 11 aa, synthetic construct
  REGION 1..11: note = Synthetic
AQVNVTRSNAQ

SEQ ID NO: 551 - protein, 11 aa, synthetic construct
  REGION 1..11: note = Synthetic
AQVPGGAGFAQ

SEQ ID NO: 552 - protein, 11 aa, synthetic construct
  REGION 1..11: note = Synthetic
AQVPSSNVSAQ

SEQ ID NO: 553 - protein, 11 aa, synthetic construct
  REGION 1..11: note = Synthetic
AQVQLVANVAQ

SEQ ID NO: 554 - protein, 11 aa, synthetic construct
  REGION 1..11: note = Synthetic
AQVQSHGVYAQ

SEQ ID NO: 555 - protein, 11 aa, synthetic construct
  REGION 1..11: note = Synthetic
AQVRSGSNIAQ

SEQ ID NO: 556 - protein, 11 aa, synthetic construct
  REGION 1..11: note = Synthetic
AQVRTAAQPAQ

SEQ ID NO: 557 - protein, 11 aa, synthetic construct
  REGION 1..11: note = Synthetic
AQVSGHEHYAQ

SEQ ID NO: 558 - protein, 11 aa, synthetic construct
  REGION 1..11: note = Synthetic
AQVSIHLTQAQ

SEQ ID NO: 559 - protein, 11 aa, synthetic construct
  REGION 1..11: note = Synthetic
AQVSNSMATAQ

SEQ ID NO: 560 - protein, 11 aa, synthetic construct
  REGION 1..11: note = Synthetic
AQVSNTLLGAQ

SEQ ID NO: 561 - protein, 11 aa, synthetic construct
  REGION 1..11: note = Synthetic
AQVSSFQSSAQ

SEQ ID NO: 562 - protein, 11 aa, synthetic construct
  REGION 1..11: note = Synthetic
AQVSSLPSPAQ

SEQ ID NO: 563 - protein, 11 aa, synthetic construct
  REGION 1..11: note = Synthetic
AQVSTANTSAQ

SEQ ID NO: 564 - protein, 11 aa, synthetic construct
  REGION 1..11: note = Synthetic
AQVSTSSLTAQ

SEQ ID NO: 565 - protein, 11 aa, synthetic construct
  REGION 1..11: note = Synthetic
AQVSVSSTAAQ

SEQ ID NO: 566 - protein, 11 aa, synthetic construct
  REGION 1..11: note = Synthetic
AQVTGSTKLAQ

SEQ ID NO: 567 - protein, 11 aa, synthetic construct
  REGION 1..11: note = Synthetic
AQVTSQIMDAQ

SEQ ID NO: 568 - protein, 11 aa, synthetic construct
  REGION 1..11: note = Synthetic
AQVTSVSYSAQ

SEQ ID NO: 569 - protein, 11 aa, synthetic construct
  REGION 1..11: note = Synthetic
AQVVSGSSHAQ

SEQ ID NO: 570 - protein, 11 aa, synthetic construct
  REGION 1..11: note = Synthetic
AQVVTDAYRAQ

SEQ ID NO: 571 - protein, 11 aa, synthetic construct
  REGION 1..11: note = Synthetic
AQWINAFKAAQ

SEQ ID NO: 572 - protein, 11 aa, synthetic construct
  REGION 1..11: note = Synthetic
AQWSQSNKAAQ

SEQ ID NO: 573 - protein, 11 aa, synthetic construct
  REGION 1..11: note = Synthetic
AQWSSRLNSAQ

SEQ ID NO: 574 - protein, 11 aa, synthetic construct
  REGION 1..11: note = Synthetic
AQYAHQYNAAQ

SEQ ID NO: 575 - protein, 11 aa, synthetic construct
  REGION 1..11: note = Synthetic
AQYDMYYDKAQ

SEQ ID NO: 576 - protein, 11 aa, synthetic construct
  REGION 1..11: note = Synthetic
AQYERSKVDAQ

SEQ ID NO: 577 - protein, 11 aa, synthetic construct
  REGION 1..11: note = Synthetic
AQYGTMIQVAQ

SEQ ID NO: 578 - protein, 11 aa, synthetic construct
  REGION 1..11: note = Synthetic
AQYHSGVGEAQ

SEQ ID NO: 579 - protein, 11 aa, synthetic construct
  REGION 1..11: note = Synthetic
AQYKATHIAAQ

SEQ ID NO: 580 - protein, 11 aa, synthetic construct
  REGION 1..11: note = Synthetic
AQYKTGNHEAQ

SEQ ID NO: 581 - protein, 11 aa, synthetic construct
  REGION 1..11: note = Synthetic
AQYLSNLMMAQ

SEQ ID NO: 582 - protein, 11 aa, synthetic construct
  REGION 1..11: note = Synthetic
AQYNGHSNNAQ

SEQ ID NO: 583 - protein, 11 aa, synthetic construct
  REGION 1..11: note = Synthetic
AQYNGQSSMAQ

SEQ ID NO: 584 - protein, 11 aa, synthetic construct
  REGION 1..11: note = Synthetic
AQYNHTRLLAQ

SEQ ID NO: 585 - protein, 11 aa, synthetic construct
  REGION 1..11: note = Synthetic
AQYNSTRASAQ

SEQ ID NO: 586 - protein, 11 aa, synthetic construct
  REGION 1..11: note = Synthetic
AQYPSSSSMAQ

SEQ ID NO: 587 - protein, 11 aa, synthetic construct
  REGION 1..11: note = Synthetic
AQYQHSSLTAQ

SEQ ID NO: 588 - protein, 11 aa, synthetic construct
  REGION 1..11: note = Synthetic
AQYQLVPRSAQ

SEQ ID NO: 589 - protein, 11 aa, synthetic construct
  REGION 1..11: note = Synthetic
AQYQTSISPAQ

SEQ ID NO: 590 - protein, 11 aa, synthetic construct
  REGION 1..11: note = Synthetic
AQYQTSTQGAQ

SEQ ID NO: 591 - protein, 11 aa, synthetic construct
  REGION 1..11: note = Synthetic
AQYSGTHTAAQ

SEQ ID NO: 592 - protein, 11 aa, synthetic construct
  REGION 1..11: note = Synthetic
AQYSSGHANAQ

SEQ ID NO: 593 - protein, 11 aa, synthetic construct
  REGION 1..11: note = Synthetic
AQYSSGLAGAQ

SEQ ID NO: 594 - protein, 11 aa, synthetic construct
  REGION 1..11: note = Synthetic
AQYVNHGLGAQ

SEQ ID NO: 595 - protein, 11 aa, synthetic construct
  REGION 1..11: note = Synthetic
AQAAGVSAGAQ

SEQ ID NO: 596 - protein, 11 aa, synthetic construct
  REGION 1..11: note = Synthetic
AQAGHNMGGAQ

SEQ ID NO: 597 - protein, 11 aa, synthetic construct
  REGION 1..11: note = Synthetic
AQANKVKDLAQ

SEQ ID NO: 598 - protein, 11 aa, synthetic construct
  REGION 1..11: note = Synthetic
AQANLTRVTAQ

SEQ ID NO: 599 - protein, 11 aa, synthetic construct
  REGION 1..11: note = Synthetic
AQANNTMKQAQ

SEQ ID NO: 600 - protein, 11 aa, synthetic construct
  REGION 1..11: note = Synthetic
AQANQTRPFAQ

SEQ ID NO: 601 - protein, 11 aa, synthetic construct
  REGION 1..11: note = Synthetic
AQANSVKNIAQ

SEQ ID NO: 602 - protein, 11 aa, synthetic construct
  REGION 1..11: note = Synthetic
AQANTTKSSAQ

SEQ ID NO: 603 - protein, 11 aa, synthetic construct
  REGION 1..11: note = Synthetic
AQANTTRQTAQ

SEQ ID NO: 604 - protein, 11 aa, synthetic construct
  REGION 1..11: note = Synthetic
AQANTVRNTAQ

SEQ ID NO: 605 - protein, 11 aa, synthetic construct
  REGION 1..11: note = Synthetic
AQARAAPQTAQ

SEQ ID NO: 606 - protein, 11 aa, synthetic construct
  REGION 1..11: note = Synthetic
AQASAGSSGAQ

SEQ ID NO: 607 - protein, 11 aa, synthetic construct
  REGION 1..11: note = Synthetic
AQASNHTSGAQ

SEQ ID NO: 608 - protein, 11 aa, synthetic construct
  REGION 1..11: note = Synthetic
AQASRSEIGAQ

SEQ ID NO: 609 - protein, 11 aa, synthetic construct
  REGION 1..11: note = Synthetic
AQASSSAREAQ

SEQ ID NO: 610 - protein, 11 aa, synthetic construct
  REGION 1..11: note = Synthetic
AQATGYSGAAQ

SEQ ID NO: 611 - protein, 11 aa, synthetic construct
  REGION 1..11: note = Synthetic
AQATHSREQAQ

SEQ ID NO: 612 - protein, 11 aa, synthetic construct
  REGION 1..11: note = Synthetic
AQATNSAVGAQ

SEQ ID NO: 613 - protein, 11 aa, synthetic construct
  REGION 1..11: note = Synthetic
AQDARPSPNAQ

SEQ ID NO: 614 - protein, 11 aa, synthetic construct
  REGION 1..11: note = Synthetic
AQDNHVRTVAQ

SEQ ID NO: 615 - protein, 11 aa, synthetic construct
  REGION 1..11: note = Synthetic
AQDNKVKTFAQ

SEQ ID NO: 616 - protein, 11 aa, synthetic construct
  REGION 1..11: note = Synthetic
AQDNMIRSIAQ

SEQ ID NO: 617 - protein, 11 aa, synthetic construct
  REGION 1..11: note = Synthetic
AQDNSTRISAQ

SEQ ID NO: 618 - protein, 11 aa, synthetic construct
  REGION 1..11: note = Synthetic
AQDNSVKHSAQ

SEQ ID NO: 619 - protein, 11 aa, synthetic construct
  REGION 1..11: note = Synthetic
AQDNSVRLTAQ

SEQ ID NO: 620 - protein, 11 aa, synthetic construct
  REGION 1..11: note = Synthetic
AQDNSVRPSAQ

SEQ ID NO: 621 - protein, 11 aa, synthetic construct
  REGION 1..11: note = Synthetic
AQDNSVRSPAQ

SEQ ID NO: 622 - protein, 11 aa, synthetic construct
  REGION 1..11: note = Synthetic
AQDNVIRSIAQ

SEQ ID NO: 623 - protein, 11 aa, synthetic construct
  REGION 1..11: note = Synthetic
AQDRAQSHSAQ

SEQ ID NO: 624 - protein, 11 aa, synthetic construct
  REGION 1..11: note = Synthetic
AQDRSQSHSAQ

SEQ ID NO: 625 - protein, 11 aa, synthetic construct
  REGION 1..11: note = Synthetic
AQDTSHRASAQ

SEQ ID NO: 626 - protein, 11 aa, synthetic construct
  REGION 1..11: note = Synthetic
AQEGGTGKVAQ

SEQ ID NO: 627 - protein, 11 aa, synthetic construct
  REGION 1..11: note = Synthetic
AQENHVRSSAQ

SEQ ID NO: 628 - protein, 11 aa, synthetic construct
  REGION 1..11: note = Synthetic
AQENKIKTNAQ

SEQ ID NO: 629 - protein, 11 aa, synthetic construct
  REGION 1..11: note = Synthetic
AQENNTRMVAQ

SEQ ID NO: 630 - protein, 11 aa, synthetic construct
  REGION 1..11: note = Synthetic
AQENQVRNTAQ

SEQ ID NO: 631 - protein, 11 aa, synthetic construct
  REGION 1..11: note = Synthetic
AQENRIINHAQ

SEQ ID NO: 632 - protein, 11 aa, synthetic construct
  REGION 1..11: note = Synthetic
AQENSTKSMAQ

SEQ ID NO: 633 - protein, 11 aa, synthetic construct
  REGION 1..11: note = Synthetic
AQENSTRVQAQ

SEQ ID NO: 634 - protein, 11 aa, synthetic construct
  REGION 1..11: note = Synthetic
AQENTTKHLAQ

SEQ ID NO: 635 - protein, 11 aa, synthetic construct
  REGION 1..11: note = Synthetic
AQENTTKSLAQ

SEQ ID NO: 636 - protein, 11 aa, synthetic construct
  REGION 1..11: note = Synthetic
AQENTVRNMAQ

SEQ ID NO: 637 - protein, 11 aa, synthetic construct
  REGION 1..11: note = Synthetic
AQETAVRSSAQ

SEQ ID NO: 638 - protein, 11 aa, synthetic construct
  REGION 1..11: note = Synthetic
AQETHNRAGAQ

SEQ ID NO: 639 - protein, 11 aa, synthetic construct
  REGION 1..11: note = Synthetic
AQEVRGGTMAQ

SEQ ID NO: 640 - protein, 11 aa, synthetic construct
  REGION 1..11: note = Synthetic
AQFNSTKVVAQ

SEQ ID NO: 641 - protein, 11 aa, synthetic construct
  REGION 1..11: note = Synthetic
AQFNSTRSVAQ

SEQ ID NO: 642 - protein, 11 aa, synthetic construct
  REGION 1..11: note = Synthetic
AQFNSVRPVAQ

SEQ ID NO: 643 - protein, 11 aa, synthetic construct
  REGION 1..11: note = Synthetic
AQGGAPPTNAQ

SEQ ID NO: 644 - protein, 11 aa, synthetic construct
  REGION 1..11: note = Synthetic
AQGNAIRNGAQ

SEQ ID NO: 645 - protein, 11 aa, synthetic construct
  REGION 1..11: note = Synthetic
AQGNAVKPFAQ

SEQ ID NO: 646 - protein, 11 aa, synthetic construct
  REGION 1..11: note = Synthetic
AQGNGTRGFAQ

SEQ ID NO: 647 - protein, 11 aa, synthetic construct
  REGION 1..11: note = Synthetic
AQGNGTRPTAQ

SEQ ID NO: 648 - protein, 11 aa, synthetic construct
  REGION 1..11: note = Synthetic
AQGNITKVFAQ

SEQ ID NO: 649 - protein, 11 aa, synthetic construct
  REGION 1..11: note = Synthetic
AQGNNSKPAAQ

SEQ ID NO: 650 - protein, 11 aa, synthetic construct
  REGION 1..11: note = Synthetic
AQGNNTKQMAQ

SEQ ID NO: 651 - protein, 11 aa, synthetic construct
  REGION 1..11: note = Synthetic
AQGNNTRLTAQ

SEQ ID NO: 652 - protein, 11 aa, synthetic construct
  REGION 1..11: note = Synthetic
AQGNQLKNTAQ

SEQ ID NO: 653 - protein, 11 aa, synthetic construct
  REGION 1..11: note = Synthetic
AQGNRIAQVAQ

SEQ ID NO: 654 - protein, 11 aa, synthetic construct
  REGION 1..11: note = Synthetic
AQGNRKPNEAQ

SEQ ID NO: 655 - protein, 11 aa, synthetic construct
  REGION 1..11: note = Synthetic
AQGNRTQPSAQ

SEQ ID NO: 656 - protein, 11 aa, synthetic construct
  REGION 1..11: note = Synthetic
AQGNRTSGGAQ

SEQ ID NO: 657 - protein, 11 aa, synthetic construct
  REGION 1..11: note = Synthetic
AQGNRVQNVAQ

SEQ ID NO: 658 - protein, 11 aa, synthetic construct
  REGION 1..11: note = Synthetic
AQGNSIINHAQ

SEQ ID NO: 659 - protein, 11 aa, synthetic construct
  REGION 1..11: note = Synthetic
AQGNSSKNLAQ

SEQ ID NO: 660 - protein, 11 aa, synthetic construct
  REGION 1..11: note = Synthetic
AQGNSVKDSAQ

SEQ ID NO: 661 - protein, 11 aa, synthetic construct
  REGION 1..11: note = Synthetic
AQGNSVKGTAQ

SEQ ID NO: 662 - protein, 11 aa, synthetic construct
  REGION 1..11: note = Synthetic
AQGNSVRISAQ

SEQ ID NO: 663 - protein, 11 aa, synthetic construct
  REGION 1..11: note = Synthetic
AQGNSVRTVAQ

SEQ ID NO: 664 - protein, 11 aa, synthetic construct
  REGION 1..11: note = Synthetic
AQGNTIKDYAQ

SEQ ID NO: 665 - protein, 11 aa, synthetic construct
  REGION 1..11: note = Synthetic
AQGNTTRHPAQ

SEQ ID NO: 666 - protein, 11 aa, synthetic construct
  REGION 1..11: note = Synthetic
AQGNTTRVPAQ

SEQ ID NO: 667 - protein, 11 aa, synthetic construct
  REGION 1..11: note = Synthetic
AQGNTVKSEAQ

SEQ ID NO: 668 - protein, 11 aa, synthetic construct
  REGION 1..11: note = Synthetic
AQGRNTSMEAQ

SEQ ID NO: 669 - protein, 11 aa, synthetic construct
  REGION 1..11: note = Synthetic
AQGRSSQGEAQ

SEQ ID NO: 670 - protein, 11 aa, synthetic construct
  REGION 1..11: note = Synthetic
AQGSRGSGAAQ

SEQ ID NO: 671 - protein, 11 aa, synthetic construct
  REGION 1..11: note = Synthetic
AQGTRSTDNAQ

SEQ ID NO: 672 - protein, 11 aa, synthetic construct
  REGION 1..11: note = Synthetic
AQGTSSNTGAQ

SEQ ID NO: 673 - protein, 11 aa, synthetic construct
  REGION 1..11: note = Synthetic
AQGVASSHQAQ

SEQ ID NO: 674 - protein, 11 aa, synthetic construct
  REGION 1..11: note = Synthetic
AQHGSSTLGAQ

SEQ ID NO: 675 - protein, 11 aa, synthetic construct
  REGION 1..11: note = Synthetic
AQHGSTFTGAQ

SEQ ID NO: 676 - protein, 11 aa, synthetic construct
  REGION 1..11: note = Synthetic
AQHIGSIPAAQ

SEQ ID NO: 677 - protein, 11 aa, synthetic construct
  REGION 1..11: note = Synthetic
AQHNNVRPVAQ

SEQ ID NO: 678 - protein, 11 aa, synthetic construct
  REGION 1..11: note = Synthetic
AQHNSTRNAAQ

SEQ ID NO: 679 - protein, 11 aa, synthetic construct
  REGION 1..11: note = Synthetic
AQHNTTKPIAQ

SEQ ID NO: 680 - protein, 11 aa, synthetic construct
  REGION 1..11: note = Synthetic
AQHSQSSKDAQ

SEQ ID NO: 681 - protein, 11 aa, synthetic construct
  REGION 1..11: note = Synthetic
AQHTSSISSAQ

SEQ ID NO: 682 - protein, 11 aa, synthetic construct
  REGION 1..11: note = Synthetic
AQIAHASSMAQ

SEQ ID NO: 683 - protein, 11 aa, synthetic construct
  REGION 1..11: note = Synthetic
AQIAHSSSGAQ

SEQ ID NO: 684 - protein, 11 aa, synthetic construct
  REGION 1..11: note = Synthetic
AQIISSNVPAQ

SEQ ID NO: 685 - protein, 11 aa, synthetic construct
  REGION 1..11: note = Synthetic
AQINATRNQAQ

SEQ ID NO: 686 - protein, 11 aa, synthetic construct
  REGION 1..11: note = Synthetic
AQINHVRDTAQ

SEQ ID NO: 687 - protein, 11 aa, synthetic construct
  REGION 1..11: note = Synthetic
AQINTTKPIAQ

SEQ ID NO: 688 - protein, 11 aa, synthetic construct
  REGION 1..11: note = Synthetic
AQIRSTTMDAQ

SEQ ID NO: 689 - protein, 11 aa, synthetic construct
  REGION 1..11: note = Synthetic
AQISAGPREAQ

SEQ ID NO: 690 - protein, 11 aa, synthetic construct
  REGION 1..11: note = Synthetic
AQLHGTSTAAQ

SEQ ID NO: 691 - protein, 11 aa, synthetic construct
  REGION 1..11: note = Synthetic
AQLNATKPIAQ

SEQ ID NO: 692 - protein, 11 aa, synthetic construct
  REGION 1..11: note = Synthetic
AQLNHTKQTAQ

SEQ ID NO: 693 - protein, 11 aa, synthetic construct
  REGION 1..11: note = Synthetic
AQLNNTKPIAQ

SEQ ID NO: 694 - protein, 11 aa, synthetic construct
  REGION 1..11: note = Synthetic
AQLNNTRPGAQ

SEQ ID NO: 695 - protein, 11 aa, synthetic construct
  REGION 1..11: note = Synthetic
AQLNSTARSAQ

SEQ ID NO: 696 - protein, 11 aa, synthetic construct
  REGION 1..11: note = Synthetic
AQLNSTKPIAQ

SEQ ID NO: 697 - protein, 11 aa, synthetic construct
  REGION 1..11: note = Synthetic
AQLNTAKPIAQ

SEQ ID NO: 698 - protein, 11 aa, synthetic construct
  REGION 1..11: note = Synthetic
AQLNTIKPIAQ

SEQ ID NO: 699 - protein, 11 aa, synthetic construct
  REGION 1..11: note = Synthetic
AQLNTSKPIAQ

SEQ ID NO: 700 - protein, 11 aa, synthetic construct
  REGION 1..11: note = Synthetic
AQLNTTKAIAQ

SEQ ID NO: 701 - protein, 11 aa, synthetic construct
  REGION 1..11: note = Synthetic
AQLNTTKHIAQ

SEQ ID NO: 702 - protein, 11 aa, synthetic construct
  REGION 1..11: note = Synthetic
AQLNTTKLAAQ

SEQ ID NO: 703 - protein, 11 aa, synthetic construct
  REGION 1..11: note = Synthetic
AQLNTTKLIAQ

SEQ ID NO: 704 - protein, 11 aa, synthetic construct
  REGION 1..11: note = Synthetic
AQLNTTKPFAQ

SEQ ID NO: 705 - protein, 11 aa, synthetic construct
  REGION 1..11: note = Synthetic
AQLNTTKPGAQ

SEQ ID NO: 706 - protein, 11 aa, synthetic construct
  REGION 1..11: note = Synthetic
AQLNTTKPIAQ

SEQ ID NO: 707 - protein, 11 aa, synthetic construct
  REGION 1..11: note = Synthetic
AQLNTTKPLAQ

SEQ ID NO: 708 - protein, 11 aa, synthetic construct
  REGION 1..11: note = Synthetic
AQLNTTKPMAQ

SEQ ID NO: 709 - protein, 11 aa, synthetic construct
  REGION 1..11: note = Synthetic
AQLNTTKPNAQ

SEQ ID NO: 710 - protein, 11 aa, synthetic construct
  REGION 1..11: note = Synthetic
AQLNTTKPSAQ

SEQ ID NO: 711 - protein, 11 aa, synthetic construct
  REGION 1..11: note = Synthetic
AQLNTTKPTAQ

SEQ ID NO: 712 - protein, 11 aa, synthetic construct
  REGION 1..11: note = Synthetic
AQLNTTKPVAQ

SEQ ID NO: 713 - protein, 11 aa, synthetic construct
  REGION 1..11: note = Synthetic
AQLNTTKSIAQ

SEQ ID NO: 714 - protein, 11 aa, synthetic construct
  REGION 1..11: note = Synthetic
AQLNTTKSVAQ

SEQ ID NO: 715 - protein, 11 aa, synthetic construct
  REGION 1..11: note = Synthetic
AQLNTTKTIAQ

SEQ ID NO: 716 - protein, 11 aa, synthetic construct
  REGION 1..11: note = Synthetic
AQLNTTRPIAQ

SEQ ID NO: 717 - protein, 11 aa, synthetic construct
  REGION 1..11: note = Synthetic
AQLNTVKPLAQ

SEQ ID NO: 718 - protein, 11 aa, synthetic construct
  REGION 1..11: note = Synthetic
AQLNYVRQVAQ

SEQ ID NO: 719 - protein, 11 aa, synthetic construct
  REGION 1..11: note = Synthetic
AQLPQHGSGAQ

SEQ ID NO: 720 - protein, 11 aa, synthetic construct
  REGION 1..11: note = Synthetic
AQLPSAHSSAQ

SEQ ID NO: 721 - protein, 11 aa, synthetic construct
  REGION 1..11: note = Synthetic
AQLSSPSTAAQ

SEQ ID NO: 722 - protein, 11 aa, synthetic construct
  REGION 1..11: note = Synthetic
AQLTNQSSAAQ

SEQ ID NO: 723 - protein, 11 aa, synthetic construct
  REGION 1..11: note = Synthetic
AQLVGTSAMAQ

SEQ ID NO: 724 - protein, 11 aa, synthetic construct
  REGION 1..11: note = Synthetic
AQLVRNTDLAQ

SEQ ID NO: 725 - protein, 11 aa, synthetic construct
  REGION 1..11: note = Synthetic
AQMHNIATPAQ

SEQ ID NO: 726 - protein, 11 aa, synthetic construct
  REGION 1..11: note = Synthetic
AQMNHTRREAQ

SEQ ID NO: 727 - protein, 11 aa, synthetic construct
  REGION 1..11: note = Synthetic
AQMNNTARPAQ

SEQ ID NO: 728 - protein, 11 aa, synthetic construct
  REGION 1..11: note = Synthetic
AQMNNTKQTAQ

SEQ ID NO: 729 - protein, 11 aa, synthetic construct
  REGION 1..11: note = Synthetic
AQMNNTRGSAQ

SEQ ID NO: 730 - protein, 11 aa, synthetic construct
  REGION 1..11: note = Synthetic
AQMNNTVRAAQ

SEQ ID NO: 731 - protein, 11 aa, synthetic construct
  REGION 1..11: note = Synthetic
AQMNSTRAAAQ

SEQ ID NO: 732 - protein, 11 aa, synthetic construct
  REGION 1..11: note = Synthetic
AQMPGHGSSAQ

SEQ ID NO: 733 - protein, 11 aa, synthetic construct
  REGION 1..11: note = Synthetic
AQMPTQHASAQ

SEQ ID NO: 734 - protein, 11 aa, synthetic construct
  REGION 1..11: note = Synthetic
AQMTHSHGSAQ

SEQ ID NO: 735 - protein, 11 aa, synthetic construct
  REGION 1..11: note = Synthetic
AQNAHNVSTAQ

SEQ ID NO: 736 - protein, 11 aa, synthetic construct
  REGION 1..11: note = Synthetic
AQNNLTRNNAQ

SEQ ID NO: 737 - protein, 11 aa, synthetic construct
  REGION 1..11: note = Synthetic
AQNNMTRAIAQ

SEQ ID NO: 738 - protein, 11 aa, synthetic construct
  REGION 1..11: note = Synthetic
AQNNMTRNNAQ

SEQ ID NO: 739 - protein, 11 aa, synthetic construct
  REGION 1..11: note = Synthetic
AQNNQIKLMAQ

SEQ ID NO: 740 - protein, 11 aa, synthetic construct
  REGION 1..11: note = Synthetic
AQNNRSPTLAQ

SEQ ID NO: 741 - protein, 11 aa, synthetic construct
  REGION 1..11: note = Synthetic
AQNNSIRLAAQ

SEQ ID NO: 742 - protein, 11 aa, synthetic construct
  REGION 1..11: note = Synthetic
AQNNSIRSSAQ

SEQ ID NO: 743 - protein, 11 aa, synthetic construct
  REGION 1..11: note = Synthetic
AQNNSTKLLAQ

SEQ ID NO: 744 - protein, 11 aa, synthetic construct
  REGION 1..11: note = Synthetic
AQNNSVRPSAQ

SEQ ID NO: 745 - protein, 11 aa, synthetic construct
  REGION 1..11: note = Synthetic
AQNNTSKNVAQ

SEQ ID NO: 746 - protein, 11 aa, synthetic construct
  REGION 1..11: note = Synthetic
AQNNTTKLAAQ

SEQ ID NO: 747 - protein, 11 aa, synthetic construct
  REGION 1..11: note = Synthetic
AQNNTTTRTAQ

SEQ ID NO: 748 - protein, 11 aa, synthetic construct
  REGION 1..11: note = Synthetic
AQNNTVKNLAQ

SEQ ID NO: 749 - protein, 11 aa, synthetic construct
  REGION 1..11: note = Synthetic
AQNNTVRPSAQ

SEQ ID NO: 750 - protein, 11 aa, synthetic construct
  REGION 1..11: note = Synthetic
AQNNVVRSTAQ

SEQ ID NO: 751 - protein, 11 aa, synthetic construct
  REGION 1..11: note = Synthetic
AQNNYVRSAAQ

SEQ ID NO: 752 - protein, 11 aa, synthetic construct
  REGION 1..11: note = Synthetic
AQNQHSSQTAQ

SEQ ID NO: 753 - protein, 11 aa, synthetic construct
  REGION 1..11: note = Synthetic
AQNRGIQSDAQ

SEQ ID NO: 754 - protein, 11 aa, synthetic construct
  REGION 1..11: note = Synthetic
AQNSHNVPSAQ

SEQ ID NO: 755 - protein, 11 aa, synthetic construct
  REGION 1..11: note = Synthetic
AQNSNNVAGAQ

SEQ ID NO: 756 - protein, 11 aa, synthetic construct
  REGION 1..11: note = Synthetic
AQNSRSVSDAQ

SEQ ID NO: 757 - protein, 11 aa, synthetic construct
  REGION 1..11: note = Synthetic
AQNSSTRTGAQ

SEQ ID NO: 758 - protein, 11 aa, synthetic construct
  REGION 1..11: note = Synthetic
AQNSTKPITAQ

SEQ ID NO: 759 - protein, 11 aa, synthetic construct
  REGION 1..11: note = Synthetic
AQNSTRPLTAQ

SEQ ID NO: 760 - protein, 11 aa, synthetic construct
  REGION 1..11: note = Synthetic
AQNTRGLVEAQ

SEQ ID NO: 761 - protein, 11 aa, synthetic construct
  REGION 1..11: note = Synthetic
AQNTTKGMAAQ

SEQ ID NO: 762 - protein, 11 aa, synthetic construct
  REGION 1..11: note = Synthetic
AQNTTRMTTAQ

SEQ ID NO: 763 - protein, 11 aa, synthetic construct
  REGION 1..11: note = Synthetic
AQNTTRSESAQ

SEQ ID NO: 764 - protein, 11 aa, synthetic construct
  REGION 1..11: note = Synthetic
AQNVRNVLDAQ

SEQ ID NO: 765 - protein, 11 aa, synthetic construct
  REGION 1..11: note = Synthetic
AQPAGRNLDAQ

SEQ ID NO: 766 - protein, 11 aa, synthetic construct
  REGION 1..11: note = Synthetic
AQPGYGNQGAQ

SEQ ID NO: 767 - protein, 11 aa, synthetic construct
  REGION 1..11: note = Synthetic
AQPMSRDTSAQ

SEQ ID NO: 768 - protein, 11 aa, synthetic construct
  REGION 1..11: note = Synthetic
AQPNAVKPMAQ

SEQ ID NO: 769 - protein, 11 aa, synthetic construct
  REGION 1..11: note = Synthetic
AQPNITRNNAQ

SEQ ID NO: 770 - protein, 11 aa, synthetic construct
  REGION 1..11: note = Synthetic
AQPNIVRSTAQ

SEQ ID NO: 771 - protein, 11 aa, synthetic construct
  REGION 1..11: note = Synthetic
AQPNQTKTTAQ

SEQ ID NO: 772 - protein, 11 aa, synthetic construct
  REGION 1..11: note = Synthetic
AQPNTTKLSAQ

SEQ ID NO: 773 - protein, 11 aa, synthetic construct
  REGION 1..11: note = Synthetic
AQPNTTKPIAQ

SEQ ID NO: 774 - protein, 11 aa, synthetic construct
  REGION 1..11: note = Synthetic
AQPNTTRSTAQ

SEQ ID NO: 775 - protein, 11 aa, synthetic construct
  REGION 1..11: note = Synthetic
AQPNVTRGFAQ

SEQ ID NO: 776 - protein, 11 aa, synthetic construct
  REGION 1..11: note = Synthetic
AQPNVTRTVAQ

SEQ ID NO: 777 - protein, 11 aa, synthetic construct
  REGION 1..11: note = Synthetic
AQQATVRTEAQ

SEQ ID NO: 778 - protein, 11 aa, synthetic construct
  REGION 1..11: note = Synthetic
AQQDRSSAVAQ

SEQ ID NO: 779 - protein, 11 aa, synthetic construct
  REGION 1..11: note = Synthetic
AQQHGVSAPAQ

SEQ ID NO: 780 - protein, 11 aa, synthetic construct
  REGION 1..11: note = Synthetic
AQQIQRSIDAQ

SEQ ID NO: 781 - protein, 11 aa, synthetic construct
  REGION 1..11: note = Synthetic
AQQNAIRQSAQ

SEQ ID NO: 782 - protein, 11 aa, synthetic construct
  REGION 1..11: note = Synthetic
AQQNATKGIAQ

SEQ ID NO: 783 - protein, 11 aa, synthetic construct
  REGION 1..11: note = Synthetic
AQQNLTRMTAQ

SEQ ID NO: 784 - protein, 11 aa, synthetic construct
  REGION 1..11: note = Synthetic
AQQNNVRPSAQ

SEQ ID NO: 785 - protein, 11 aa, synthetic construct
  REGION 1..11: note = Synthetic
AQQNSIRSVAQ

SEQ ID NO: 786 - protein, 11 aa, synthetic construct
  REGION 1..11: note = Synthetic
AQQNSTRSLAQ

SEQ ID NO: 787 - protein, 11 aa, synthetic construct
  REGION 1..11: note = Synthetic
AQQNTTRTSAQ

SEQ ID NO: 788 - protein, 11 aa, synthetic construct
  REGION 1..11: note = Synthetic
AQQNYTKVAAQ

SEQ ID NO: 789 - protein, 11 aa, synthetic construct
  REGION 1..11: note = Synthetic
AQQRIPSNTAQ

SEQ ID NO: 790 - protein, 11 aa, synthetic construct
  REGION 1..11: note = Synthetic
AQQRTSSQEAQ

SEQ ID NO: 791 - protein, 11 aa, synthetic construct
  REGION 1..11: note = Synthetic
AQQSSSRLGAQ

SEQ ID NO: 792 - protein, 11 aa, synthetic construct
  REGION 1..11: note = Synthetic
AQQVRSDMGAQ

SEQ ID NO: 793 - protein, 11 aa, synthetic construct
  REGION 1..11: note = Synthetic
AQRDGLILIAQ

SEQ ID NO: 794 - protein, 11 aa, synthetic construct
  REGION 1..11: note = Synthetic
AQRDNIILIAQ

SEQ ID NO: 795 - protein, 11 aa, synthetic construct
  REGION 1..11: note = Synthetic
AQRDSTMHAAQ

SEQ ID NO: 796 - protein, 11 aa, synthetic construct
  REGION 1..11: note = Synthetic
AQRDTHVFHAQ

SEQ ID NO: 797 - protein, 11 aa, synthetic construct
  REGION 1..11: note = Synthetic
AQRGDLHFQAQ

SEQ ID NO: 798 - protein, 11 aa, synthetic construct
  REGION 1..11: note = Synthetic
AQRLSQSSDAQ

SEQ ID NO: 799 - protein, 11 aa, synthetic construct
  REGION 1..11: note = Synthetic
AQRSGLILFAQ

SEQ ID NO: 800 - protein, 11 aa, synthetic construct
  REGION 1..11: note = Synthetic
AQRSGSHFVAQ

SEQ ID NO: 801 - protein, 11 aa, synthetic construct
  REGION 1..11: note = Synthetic
AQRVGQSVSAQ

SEQ ID NO: 802 - protein, 11 aa, synthetic construct
  REGION 1..11: note = Synthetic
AQRVVGPLFAQ

SEQ ID NO: 803 - protein, 11 aa, synthetic construct
  REGION 1..11: note = Synthetic
AQSDRGNHSAQ

SEQ ID NO: 804 - protein, 11 aa, synthetic construct
  REGION 1..11: note = Synthetic
AQSDSRNASAQ

SEQ ID NO: 805 - protein, 11 aa, synthetic construct
  REGION 1..11: note = Synthetic
AQSESARGNAQ

SEQ ID NO: 806 - protein, 11 aa, synthetic construct
  REGION 1..11: note = Synthetic
AQSGHNASGAQ

SEQ ID NO: 807 - protein, 11 aa, synthetic construct
  REGION 1..11: note = Synthetic
AQSGSHTNSAQ

SEQ ID NO: 808 - protein, 11 aa, synthetic construct
  REGION 1..11: note = Synthetic
AQSGSSRGEAQ

SEQ ID NO: 809 - protein, 11 aa, synthetic construct
  REGION 1..11: note = Synthetic
AQSLSQPERAQ

SEQ ID NO: 810 - protein, 11 aa, synthetic construct
  REGION 1..11: note = Synthetic
AQSMGHSHAAQ

SEQ ID NO: 811 - protein, 11 aa, synthetic construct
  REGION 1..11: note = Synthetic
AQSMSYGSSAQ

SEQ ID NO: 812 - protein, 11 aa, synthetic construct
  REGION 1..11: note = Synthetic
AQSNAAKPVAQ

SEQ ID NO: 813 - protein, 11 aa, synthetic construct
  REGION 1..11: note = Synthetic
AQSNATTRAAQ

SEQ ID NO: 814 - protein, 11 aa, synthetic construct
  REGION 1..11: note = Synthetic
AQSNDIRNNAQ

SEQ ID NO: 815 - protein, 11 aa, synthetic construct
  REGION 1..11: note = Synthetic
AQSNDTKNTAQ

SEQ ID NO: 816 - protein, 11 aa, synthetic construct
  REGION 1..11: note = Synthetic
AQSNGIRNTAQ

SEQ ID NO: 817 - protein, 11 aa, synthetic construct
  REGION 1..11: note = Synthetic
AQSNHTTRSAQ

SEQ ID NO: 818 - protein, 11 aa, synthetic construct
  REGION 1..11: note = Synthetic
AQSNLVRQTAQ

SEQ ID NO: 819 - protein, 11 aa, synthetic construct
  REGION 1..11: note = Synthetic
AQSNNAKPSAQ

SEQ ID NO: 820 - protein, 11 aa, synthetic construct
  REGION 1..11: note = Synthetic
AQSNNSSRPAQ

SEQ ID NO: 821 - protein, 11 aa, synthetic construct
  REGION 1..11: note = Synthetic
AQSNQTRGNAQ

SEQ ID NO: 822 - protein, 11 aa, synthetic construct
  REGION 1..11: note = Synthetic
AQSNSTKMIAQ

SEQ ID NO: 823 - protein, 11 aa, synthetic construct
  REGION 1..11: note = Synthetic
AQSNSTKNAAQ

SEQ ID NO: 824 - protein, 11 aa, synthetic construct
  REGION 1..11: note = Synthetic
AQSNSTKNVAQ

SEQ ID NO: 825 - protein, 11 aa, synthetic construct
  REGION 1..11: note = Synthetic
AQSNSTKSLAQ

SEQ ID NO: 826 - protein, 11 aa, synthetic construct
  REGION 1..11: note = Synthetic
AQSNSTRSSAQ

SEQ ID NO: 827 - protein, 11 aa, synthetic construct
  REGION 1..11: note = Synthetic
AQSNSVKAPAQ

SEQ ID NO: 828 - protein, 11 aa, synthetic construct
  REGION 1..11: note = Synthetic
AQSNSVKERAQ

SEQ ID NO: 829 - protein, 11 aa, synthetic construct
  REGION 1..11: note = Synthetic
AQSNSVKITAQ

SEQ ID NO: 830 - protein, 11 aa, synthetic construct
  REGION 1..11: note = Synthetic
AQSNSVKSFAQ

SEQ ID NO: 831 - protein, 11 aa, synthetic construct
  REGION 1..11: note = Synthetic
AQSNSVKTVAQ

SEQ ID NO: 832 - protein, 11 aa, synthetic construct
  REGION 1..11: note = Synthetic
AQSNSVRLVAQ

SEQ ID NO: 833 - protein, 11 aa, synthetic construct
  REGION 1..11: note = Synthetic
AQSNTTKPIAQ

SEQ ID NO: 834 - protein, 11 aa, synthetic construct
  REGION 1..11: note = Synthetic
AQSNTTRPMAQ

SEQ ID NO: 835 - protein, 11 aa, synthetic construct
  REGION 1..11: note = Synthetic
AQSNTTRQTAQ

SEQ ID NO: 836 - protein, 11 aa, synthetic construct
  REGION 1..11: note = Synthetic
AQSNTTTRAAQ

SEQ ID NO: 837 - protein, 11 aa, synthetic construct
  REGION 1..11: note = Synthetic
AQSNTVKAMAQ

SEQ ID NO: 838 - protein, 11 aa, synthetic construct
  REGION 1..11: note = Synthetic
AQSNTVKNAAQ

SEQ ID NO: 839 - protein, 11 aa, synthetic construct
  REGION 1..11: note = Synthetic
AQSNTVRNSAQ

SEQ ID NO: 840 - protein, 11 aa, synthetic construct
  REGION 1..11: note = Synthetic
AQSNTVRSSAQ

SEQ ID NO: 841 - protein, 11 aa, synthetic construct
  REGION 1..11: note = Synthetic
AQSNVIRNSAQ

SEQ ID NO: 842 - protein, 11 aa, synthetic construct
  REGION 1..11: note = Synthetic
AQSNYTRSPAQ

SEQ ID NO: 843 - protein, 11 aa, synthetic construct
  REGION 1..11: note = Synthetic
AQSPSRHTEAQ

SEQ ID NO: 844 - protein, 11 aa, synthetic construct
  REGION 1..11: note = Synthetic
AQSQHASSAAQ

SEQ ID NO: 845 - protein, 11 aa, synthetic construct
  REGION 1..11: note = Synthetic
AQSQSSSGSAQ

SEQ ID NO: 846 - protein, 11 aa, synthetic construct
  REGION 1..11: note = Synthetic
AQSSATRVDAQ

SEQ ID NO: 847 - protein, 11 aa, synthetic construct
  REGION 1..11: note = Synthetic
AQSSGYAQQAQ

SEQ ID NO: 848 - protein, 11 aa, synthetic construct
  REGION 1..11: note = Synthetic
AQSSNSGREAQ

SEQ ID NO: 849 - protein, 11 aa, synthetic construct
  REGION 1..11: note = Synthetic
AQSSSGGTGAQ

SEQ ID NO: 850 - protein, 11 aa, synthetic construct
  REGION 1..11: note = Synthetic
AQSTAIRQDAQ

SEQ ID NO: 851 - protein, 11 aa, synthetic construct
  REGION 1..11: note = Synthetic
AQSTQTRAAAQ

SEQ ID NO: 852 - protein, 11 aa, synthetic construct
  REGION 1..11: note = Synthetic
AQSTQVRSEAQ

SEQ ID NO: 853 - protein, 11 aa, synthetic construct
  REGION 1..11: note = Synthetic
AQSTRGNDNAQ

SEQ ID NO: 854 - protein, 11 aa, synthetic construct
  REGION 1..11: note = Synthetic
AQSTSTHGAAQ

SEQ ID NO: 855 - protein, 11 aa, synthetic construct
  REGION 1..11: note = Synthetic
AQTGVRSGDAQ

SEQ ID NO: 856 - protein, 11 aa, synthetic construct
  REGION 1..11: note = Synthetic
AQTMRQTNDAQ

SEQ ID NO: 857 - protein, 11 aa, synthetic construct
  REGION 1..11: note = Synthetic
AQTNHTREVAQ

SEQ ID NO: 858 - protein, 11 aa, synthetic construct
  REGION 1..11: note = Synthetic
AQTNHVKTMAQ

SEQ ID NO: 859 - protein, 11 aa, synthetic construct
  REGION 1..11: note = Synthetic
AQTNHVRSAAQ

SEQ ID NO: 860 - protein, 11 aa, synthetic construct
  REGION 1..11: note = Synthetic
AQTNLVKPTAQ

SEQ ID NO: 861 - protein, 11 aa, synthetic construct
  REGION 1..11: note = Synthetic
AQTNMTRTTAQ

SEQ ID NO: 862 - protein, 11 aa, synthetic construct
  REGION 1..11: note = Synthetic
AQTNNATRPAQ

SEQ ID NO: 863 - protein, 11 aa, synthetic construct
  REGION 1..11: note = Synthetic
AQTNNAVRAAQ

SEQ ID NO: 864 - protein, 11 aa, synthetic construct
  REGION 1..11: note = Synthetic
AQTNNTKSTAQ

SEQ ID NO: 865 - protein, 11 aa, synthetic construct
  REGION 1..11: note = Synthetic
AQTNNTLRSAQ

SEQ ID NO: 866 - protein, 11 aa, synthetic construct
  REGION 1..11: note = Synthetic
AQTNNTSPGAQ

SEQ ID NO: 867 - protein, 11 aa, synthetic construct
  REGION 1..11: note = Synthetic
AQTNNVRAPAQ

SEQ ID NO: 868 - protein, 11 aa, synthetic construct
  REGION 1..11: note = Synthetic
AQTNSIKSTAQ

SEQ ID NO: 869 - protein, 11 aa, synthetic construct
  REGION 1..11: note = Synthetic
AQTNSIRPIAQ

SEQ ID NO: 870 - protein, 11 aa, synthetic construct
  REGION 1..11: note = Synthetic
AQTNSVKLIAQ

SEQ ID NO: 871 - protein, 11 aa, synthetic construct
  REGION 1..11: note = Synthetic
AQTNTTKSMAQ

SEQ ID NO: 872 - protein, 11 aa, synthetic construct
  REGION 1..11: note = Synthetic
AQTNTVRASAQ

SEQ ID NO: 873 - protein, 11 aa, synthetic construct
  REGION 1..11: note = Synthetic
AQTNTVRQTAQ

SEQ ID NO: 874 - protein, 11 aa, synthetic construct
  REGION 1..11: note = Synthetic
AQTRGASNEAQ

SEQ ID NO: 875 - protein, 11 aa, synthetic construct
  REGION 1..11: note = Synthetic
AQTSGRSDAAQ

SEQ ID NO: 876 - protein, 11 aa, synthetic construct
  REGION 1..11: note = Synthetic
AQTVSDRSGAQ

SEQ ID NO: 877 - protein, 11 aa, synthetic construct
  REGION 1..11: note = Synthetic
AQVGHSAVSAQ

SEQ ID NO: 878 - protein, 11 aa, synthetic construct
  REGION 1..11: note = Synthetic
AQVHNRADVAQ

SEQ ID NO: 879 - protein, 11 aa, synthetic construct
  REGION 1..11: note = Synthetic
AQVHSGPVSAQ

SEQ ID NO: 880 - protein, 11 aa, synthetic construct
  REGION 1..11: note = Synthetic
AQVNATRNVAQ

SEQ ID NO: 881 - protein, 11 aa, synthetic construct
  REGION 1..11: note = Synthetic
AQVNHTKGLAQ

SEQ ID NO: 882 - protein, 11 aa, synthetic construct
  REGION 1..11: note = Synthetic
AQVNSLGNNAQ

SEQ ID NO: 883 - protein, 11 aa, synthetic construct
  REGION 1..11: note = Synthetic
AQVNSTKSMAQ

SEQ ID NO: 884 - protein, 11 aa, synthetic construct
  REGION 1..11: note = Synthetic
AQVNSTKSTAQ

SEQ ID NO: 885 - protein, 11 aa, synthetic construct
  REGION 1..11: note = Synthetic
AQVNSTRGNAQ

SEQ ID NO: 886 - protein, 11 aa, synthetic construct
  REGION 1..11: note = Synthetic
AQVNTTKPIAQ

SEQ ID NO: 887 - protein, 11 aa, synthetic construct
  REGION 1..11: note = Synthetic
AQVNTTQSGAQ

SEQ ID NO: 888 - protein, 11 aa, synthetic construct
  REGION 1..11: note = Synthetic
AQVSDTRAGAQ

SEQ ID NO: 889 - protein, 11 aa, synthetic construct
  REGION 1..11: note = Synthetic
AQVSHSLASAQ

SEQ ID NO: 890 - protein, 11 aa, synthetic construct
  REGION 1..11: note = Synthetic
AQVSSSGMTAQ

SEQ ID NO: 891 - protein, 11 aa, synthetic construct
  REGION 1..11: note = Synthetic
AQVSVTRSDAQ

SEQ ID NO: 892 - protein, 11 aa, synthetic construct
  REGION 1..11: note = Synthetic
AQWAANTSGAQ

SEQ ID NO: 893 - protein, 11 aa, synthetic construct
  REGION 1..11: note = Synthetic
AQYAGVSSPAQ

SEQ ID NO: 894 - protein, 11 aa, synthetic construct
  REGION 1..11: note = Synthetic
AQYGSHGVGAQ

SEQ ID NO: 895 - protein, 11 aa, synthetic construct
  REGION 1..11: note = Synthetic
AQYNSTKNGAQ

SEQ ID NO: 896 - protein, 11 aa, synthetic construct
  REGION 1..11: note = Synthetic
AQYNSTKNMAQ

SEQ ID NO: 897 - protein, 11 aa, synthetic construct
  REGION 1..11: note = Synthetic
AQYNSTKSHAQ

SEQ ID NO: 898 - protein, 11 aa, synthetic construct
  REGION 1..11: note = Synthetic
AQYNTTKPIAQ

SEQ ID NO: 899 - protein, 11 aa, synthetic construct
  REGION 1..11: note = Synthetic
AQAAHNKDSAQ

SEQ ID NO: 900 - protein, 11 aa, synthetic construct
  REGION 1..11: note = Synthetic
AQADARNTSAQ

SEQ ID NO: 901 - protein, 11 aa, synthetic construct
  REGION 1..11: note = Synthetic
AQAESRGSAAQ

SEQ ID NO: 902 - protein, 11 aa, synthetic construct
  REGION 1..11: note = Synthetic
AQAESRIGSAQ

SEQ ID NO: 903 - protein, 11 aa, synthetic construct
  REGION 1..11: note = Synthetic
AQAESRTMPAQ

SEQ ID NO: 904 - protein, 11 aa, synthetic construct
  REGION 1..11: note = Synthetic
AQAHAATSAAQ

SEQ ID NO: 905 - protein, 11 aa, synthetic construct
  REGION 1..11: note = Synthetic
AQAISLRGDAQ

SEQ ID NO: 906 - protein, 11 aa, synthetic construct
  REGION 1..11: note = Synthetic
AQAKGSTLLAQ

SEQ ID NO: 907 - protein, 11 aa, synthetic construct
  REGION 1..11: note = Synthetic
AQALARSVSAQ

SEQ ID NO: 908 - protein, 11 aa, synthetic construct
  REGION 1..11: note = Synthetic
AQALGQSRVAQ

SEQ ID NO: 909 - protein, 11 aa, synthetic construct
  REGION 1..11: note = Synthetic
AQALSHSSGAQ

SEQ ID NO: 910 - protein, 11 aa, synthetic construct
  REGION 1..11: note = Synthetic
AQANHTRVAAQ

SEQ ID NO: 911 - protein, 11 aa, synthetic construct
  REGION 1..11: note = Synthetic
AQAQGFNLLAQ

SEQ ID NO: 912 - protein, 11 aa, synthetic construct
  REGION 1..11: note = Synthetic
AQARTGPMLAQ

SEQ ID NO: 913 - protein, 11 aa, synthetic construct
  REGION 1..11: note = Synthetic
AQARTHLQDAQ

SEQ ID NO: 914 - protein, 11 aa, synthetic construct
  REGION 1..11: note = Synthetic
AQARVGATAAQ

SEQ ID NO: 915 - protein, 11 aa, synthetic construct
  REGION 1..11: note = Synthetic
AQARVGTNVAQ

SEQ ID NO: 916 - protein, 11 aa, synthetic construct
  REGION 1..11: note = Synthetic
AQASHSRDAAQ

SEQ ID NO: 917 - protein, 11 aa, synthetic construct
  REGION 1..11: note = Synthetic
AQASSNKTAAQ

SEQ ID NO: 918 - protein, 11 aa, synthetic construct
  REGION 1..11: note = Synthetic
AQASSSTAGAQ

SEQ ID NO: 919 - protein, 11 aa, synthetic construct
  REGION 1..11: note = Synthetic
AQATARNAVAQ

SEQ ID NO: 920 - protein, 11 aa, synthetic construct
  REGION 1..11: note = Synthetic
AQATGRLPEAQ

SEQ ID NO: 921 - protein, 11 aa, synthetic construct
  REGION 1..11: note = Synthetic
AQATRNTDMAQ

SEQ ID NO: 922 - protein, 11 aa, synthetic construct
  REGION 1..11: note = Synthetic
AQATSIRGPAQ

SEQ ID NO: 923 - protein, 11 aa, synthetic construct
  REGION 1..11: note = Synthetic
AQATVSSRIAQ

SEQ ID NO: 924 - protein, 11 aa, synthetic construct
  REGION 1..11: note = Synthetic
AQAVNKPLSAQ

SEQ ID NO: 925 - protein, 11 aa, synthetic construct
  REGION 1..11: note = Synthetic
AQAVPQSAGAQ

SEQ ID NO: 926 - protein, 11 aa, synthetic construct
  REGION 1..11: note = Synthetic
AQAVPSHHGAQ

SEQ ID NO: 927 - protein, 11 aa, synthetic construct
  REGION 1..11: note = Synthetic
AQAVRGAMNAQ

SEQ ID NO: 928 - protein, 11 aa, synthetic construct
  REGION 1..11: note = Synthetic
AQAVSGIRIAQ

SEQ ID NO: 929 - protein, 11 aa, synthetic construct
  REGION 1..11: note = Synthetic
AQAVSRSVDAQ

SEQ ID NO: 930 - protein, 11 aa, synthetic construct
  REGION 1..11: note = Synthetic
AQAVWPTRNAQ

SEQ ID NO: 931 - protein, 11 aa, synthetic construct
  REGION 1..11: note = Synthetic
AQAYARGESAQ

SEQ ID NO: 932 - protein, 11 aa, synthetic construct
  REGION 1..11: note = Synthetic
AQAYGETFWAQ

SEQ ID NO: 933 - protein, 11 aa, synthetic construct
  REGION 1..11: note = Synthetic
AQCLGPCLQAQ

SEQ ID NO: 934 - protein, 11 aa, synthetic construct
  REGION 1..11: note = Synthetic
AQDGIRLTPAQ

SEQ ID NO: 935 - protein, 11 aa, synthetic construct
  REGION 1..11: note = Synthetic
AQDGRTVSMAQ

SEQ ID NO: 936 - protein, 11 aa, synthetic construct
  REGION 1..11: note = Synthetic
AQDGSTRMGAQ

SEQ ID NO: 937 - protein, 11 aa, synthetic construct
  REGION 1..11: note = Synthetic
AQDIRSTASAQ

SEQ ID NO: 938 - protein, 11 aa, synthetic construct
  REGION 1..11: note = Synthetic
AQDLSTARSAQ

SEQ ID NO: 939 - protein, 11 aa, synthetic construct
  REGION 1..11: note = Synthetic
AQDLSTRVAAQ

SEQ ID NO: 940 - protein, 11 aa, synthetic construct
  REGION 1..11: note = Synthetic
AQDMSRSTGAQ

SEQ ID NO: 941 - protein, 11 aa, synthetic construct
  REGION 1..11: note = Synthetic
AQDNVHSRGAQ

SEQ ID NO: 942 - protein, 11 aa, synthetic construct
  REGION 1..11: note = Synthetic
AQDQHNKSQAQ

SEQ ID NO: 943 - protein, 11 aa, synthetic construct
  REGION 1..11: note = Synthetic
AQDQKNATGAQ

SEQ ID NO: 944 - protein, 11 aa, synthetic construct
  REGION 1..11: note = Synthetic
AQDRAQSNMAQ

SEQ ID NO: 945 - protein, 11 aa, synthetic construct
  REGION 1..11: note = Synthetic
AQDRGTSQTAQ

SEQ ID NO: 946 - protein, 11 aa, synthetic construct
  REGION 1..11: note = Synthetic
AQDRRSSDLAQ

SEQ ID NO: 947 - protein, 11 aa, synthetic construct
  REGION 1..11: note = Synthetic
AQDRSHGHNAQ

SEQ ID NO: 948 - protein, 11 aa, synthetic construct
  REGION 1..11: note = Synthetic
AQDRSNTSNAQ

SEQ ID NO: 949 - protein, 11 aa, synthetic construct
  REGION 1..11: note = Synthetic
AQDRSTTHAAQ

SEQ ID NO: 950 - protein, 11 aa, synthetic construct
  REGION 1..11: note = Synthetic
AQDSLPEPYAQ

SEQ ID NO: 951 - protein, 11 aa, synthetic construct
  REGION 1..11: note = Synthetic
AQDSNRAVAAQ

SEQ ID NO: 952 - protein, 11 aa, synthetic construct
  REGION 1..11: note = Synthetic
AQDSNRPSNAQ

SEQ ID NO: 953 - protein, 11 aa, synthetic construct
  REGION 1..11: note = Synthetic
AQDSRQSKEAQ

SEQ ID NO: 954 - protein, 11 aa, synthetic construct
  REGION 1..11: note = Synthetic
AQDSSNRTGAQ

SEQ ID NO: 955 - protein, 11 aa, synthetic construct
  REGION 1..11: note = Synthetic
AQDSYTRSAAQ

SEQ ID NO: 956 - protein, 11 aa, synthetic construct
  REGION 1..11: note = Synthetic
AQDTHARGLAQ

SEQ ID NO: 957 - protein, 11 aa, synthetic construct
  REGION 1..11: note = Synthetic
AQDTRQSFSAQ

SEQ ID NO: 958 - protein, 11 aa, synthetic construct
  REGION 1..11: note = Synthetic
AQEASIRSGAQ

SEQ ID NO: 959 - protein, 11 aa, synthetic construct
  REGION 1..11: note = Synthetic
AQEGNISRPAQ

SEQ ID NO: 960 - protein, 11 aa, synthetic construct
  REGION 1..11: note = Synthetic
AQELTMPPYAQ

SEQ ID NO: 961 - protein, 11 aa, synthetic construct
  REGION 1..11: note = Synthetic
AQEMSKANMAQ

SEQ ID NO: 962 - protein, 11 aa, synthetic construct
  REGION 1..11: note = Synthetic
AQENQRSGAAQ

SEQ ID NO: 963 - protein, 11 aa, synthetic construct
  REGION 1..11: note = Synthetic
AQERGKNLEAQ

SEQ ID NO: 964 - protein, 11 aa, synthetic construct
  REGION 1..11: note = Synthetic
AQERGTVSTAQ

SEQ ID NO: 965 - protein, 11 aa, synthetic construct
  REGION 1..11: note = Synthetic
AQERNALREAQ

SEQ ID NO: 966 - protein, 11 aa, synthetic construct
  REGION 1..11: note = Synthetic
AQESGQRNQAQ

SEQ ID NO: 967 - protein, 11 aa, synthetic construct
  REGION 1..11: note = Synthetic
AQESNSYRPAQ

SEQ ID NO: 968 - protein, 11 aa, synthetic construct
  REGION 1..11: note = Synthetic
AQESRATTPAQ

SEQ ID NO: 969 - protein, 11 aa, synthetic construct
  REGION 1..11: note = Synthetic
AQESTKYHAAQ

SEQ ID NO: 970 - protein, 11 aa, synthetic construct
  REGION 1..11: note = Synthetic
AQESTRISGAQ

SEQ ID NO: 971 - protein, 11 aa, synthetic construct
  REGION 1..11: note = Synthetic
AQETGRYNPAQ

SEQ ID NO: 972 - protein, 11 aa, synthetic construct
  REGION 1..11: note = Synthetic
AQETTSWVFAQ

SEQ ID NO: 973 - protein, 11 aa, synthetic construct
  REGION 1..11: note = Synthetic
AQEVNRSSYAQ

SEQ ID NO: 974 - protein, 11 aa, synthetic construct
  REGION 1..11: note = Synthetic
AQEVSRGKDAQ

SEQ ID NO: 975 - protein, 11 aa, synthetic construct
  REGION 1..11: note = Synthetic
AQEYGKATAAQ

SEQ ID NO: 976 - protein, 11 aa, synthetic construct
  REGION 1..11: note = Synthetic
AQFAGMRTAAQ

SEQ ID NO: 977 - protein, 11 aa, synthetic construct
  REGION 1..11: note = Synthetic
AQFDSVRNSAQ

SEQ ID NO: 978 - protein, 11 aa, synthetic construct
  REGION 1..11: note = Synthetic
AQFGSRDGSAQ

SEQ ID NO: 979 - protein, 11 aa, synthetic construct
  REGION 1..11: note = Synthetic
AQFGSRQPEAQ

SEQ ID NO: 980 - protein, 11 aa, synthetic construct
  REGION 1..11: note = Synthetic
AQFHNTRSEAQ

SEQ ID NO: 981 - protein, 11 aa, synthetic construct
  REGION 1..11: note = Synthetic
AQFNSDRPGAQ

SEQ ID NO: 982 - protein, 11 aa, synthetic construct
  REGION 1..11: note = Synthetic
AQFNSTRIVAQ

SEQ ID NO: 983 - protein, 11 aa, synthetic construct
  REGION 1..11: note = Synthetic
AQFNVSSTGAQ

SEQ ID NO: 984 - protein, 11 aa, synthetic construct
  REGION 1..11: note = Synthetic
AQFQVATRDAQ

SEQ ID NO: 985 - protein, 11 aa, synthetic construct
  REGION 1..11: note = Synthetic
AQFSNRTASAQ

SEQ ID NO: 986 - protein, 11 aa, synthetic construct
  REGION 1..11: note = Synthetic
AQFSRGVNEAQ

SEQ ID NO: 987 - protein, 11 aa, synthetic construct
  REGION 1..11: note = Synthetic
AQGAGALRGAQ

SEQ ID NO: 988 - protein, 11 aa, synthetic construct
  REGION 1..11: note = Synthetic
AQGAGRTLEAQ

SEQ ID NO: 989 - protein, 11 aa, synthetic construct
  REGION 1..11: note = Synthetic
AQGDRLTHGAQ

SEQ ID NO: 990 - protein, 11 aa, synthetic construct
  REGION 1..11: note = Synthetic
AQGFGHSVSAQ

SEQ ID NO: 991 - protein, 11 aa, synthetic construct
  REGION 1..11: note = Synthetic
AQGGTAARGAQ

SEQ ID NO: 992 - protein, 11 aa, synthetic construct
  REGION 1..11: note = Synthetic
AQGGVRNAEAQ

SEQ ID NO: 993 - protein, 11 aa, synthetic construct
  REGION 1..11: note = Synthetic
AQGHHRTVEAQ

SEQ ID NO: 994 - protein, 11 aa, synthetic construct
  REGION 1..11: note = Synthetic
AQGHSANLGAQ

SEQ ID NO: 995 - protein, 11 aa, synthetic construct
  REGION 1..11: note = Synthetic
AQGIRNQAEAQ

SEQ ID NO: 996 - protein, 11 aa, synthetic construct
  REGION 1..11: note = Synthetic
AQGLGTSSNAQ

SEQ ID NO: 997 - protein, 11 aa, synthetic construct
  REGION 1..11: note = Synthetic
AQGLNRLTPAQ

SEQ ID NO: 998 - protein, 11 aa, synthetic construct
  REGION 1..11: note = Synthetic
AQGLRSSDSAQ

SEQ ID NO: 999 - protein, 11 aa, synthetic construct
  REGION 1..11: note = Synthetic
AQGMRTSNDAQ

SEQ ID NO: 1000 - protein, 11 aa, synthetic construct
  REGION 1..11: note = Synthetic
AQGNGSRMAAQ

SEQ ID NO: 1001 - protein, 11 aa, synthetic construct
  REGION 1..11: note = Synthetic
AQGNRIAREAQ

SEQ ID NO: 1002 - protein, 11 aa, synthetic construct
  REGION 1..11: note = Synthetic
AQGRSLTTDAQ

SEQ ID NO: 1003 - protein, 11 aa, synthetic construct
  REGION 1..11: note = Synthetic
AQGSKPSQMAQ

SEQ ID NO: 1004 - protein, 11 aa, synthetic construct
  REGION 1..11: note = Synthetic
AQGSYANSQAQ

SEQ ID NO: 1005 - protein, 11 aa, synthetic construct
  REGION 1..11: note = Synthetic
AQGVAKGNEAQ

SEQ ID NO: 1006 - protein, 11 aa, synthetic construct
  REGION 1..11: note = Synthetic
AQGVMGMRNAQ

SEQ ID NO: 1007 - protein, 11 aa, synthetic construct
  REGION 1..11: note = Synthetic
AQGVRSGLSAQ

SEQ ID NO: 1008 - protein, 11 aa, synthetic construct
  REGION 1..11: note = Synthetic
AQGVRSHDAAQ

SEQ ID NO: 1009 - protein, 11 aa, synthetic construct
  REGION 1..11: note = Synthetic
AQGVSNRANAQ

SEQ ID NO: 1010 - protein, 11 aa, synthetic construct
  REGION 1..11: note = Synthetic
AQGVSRATPAQ

SEQ ID NO: 1011 - protein, 11 aa, synthetic construct
  REGION 1..11: note = Synthetic
AQGVTLGSRAQ

SEQ ID NO: 1012 - protein, 11 aa, synthetic construct
  REGION 1..11: note = Synthetic
AQGYNSTPGAQ

SEQ ID NO: 1013 - protein, 11 aa, synthetic construct
  REGION 1..11: note = Synthetic
AQGYSGATGAQ

SEQ ID NO: 1014 - protein, 11 aa, synthetic construct
  REGION 1..11: note = Synthetic
AQGYSHSSGAQ

SEQ ID NO: 1015 - protein, 11 aa, synthetic construct
  REGION 1..11: note = Synthetic
AQHFVDSGLAQ

SEQ ID NO: 1016 - protein, 11 aa, synthetic construct
  REGION 1..11: note = Synthetic
AQHGTSRPDAQ

SEQ ID NO: 1017 - protein, 11 aa, synthetic construct
  REGION 1..11: note = Synthetic
AQHIVTHDLAQ

SEQ ID NO: 1018 - protein, 11 aa, synthetic construct
  REGION 1..11: note = Synthetic
AQHLVDRSLAQ

SEQ ID NO: 1019 - protein, 11 aa, synthetic construct
  REGION 1..11: note = Synthetic
AQHMSDSPHAQ

SEQ ID NO: 1020 - protein, 11 aa, synthetic construct
  REGION 1..11: note = Synthetic
AQHMVPSLLAQ

SEQ ID NO: 1021 - protein, 11 aa, synthetic construct
  REGION 1..11: note = Synthetic
AQHNLPVLHAQ

SEQ ID NO: 1022 - protein, 11 aa, synthetic construct
  REGION 1..11: note = Synthetic
AQHNPTRADAQ

SEQ ID NO: 1023 - protein, 11 aa, synthetic construct
  REGION 1..11: note = Synthetic
AQHPVQSSLAQ

SEQ ID NO: 1024 - protein, 11 aa, synthetic construct
  REGION 1..11: note = Synthetic
AQHQAKLQNAQ

SEQ ID NO: 1025 - protein, 11 aa, synthetic construct
  REGION 1..11: note = Synthetic
AQHQIAYDLAQ

SEQ ID NO: 1026 - protein, 11 aa, synthetic construct
  REGION 1..11: note = Synthetic
AQHQVPSLLAQ

SEQ ID NO: 1027 - protein, 11 aa, synthetic construct
  REGION 1..11: note = Synthetic
AQHRIATVEAQ

SEQ ID NO: 1028 - protein, 11 aa, synthetic construct
  REGION 1..11: note = Synthetic
AQHRVSEHFAQ

SEQ ID NO: 1029 - protein, 11 aa, synthetic construct
  REGION 1..11: note = Synthetic
AQHSSIGSSAQ

SEQ ID NO: 1030 - protein, 11 aa, synthetic construct
  REGION 1..11: note = Synthetic
AQHSVSHHFAQ

SEQ ID NO: 1031 - protein, 11 aa, synthetic construct
  REGION 1..11: note = Synthetic
AQHYLSTDFAQ

SEQ ID NO: 1032 - protein, 11 aa, synthetic construct
  REGION 1..11: note = Synthetic
AQIASRGLAAQ

SEQ ID NO: 1033 - protein, 11 aa, synthetic construct
  REGION 1..11: note = Synthetic
AQIDNRHNGAQ

SEQ ID NO: 1034 - protein, 11 aa, synthetic construct
  REGION 1..11: note = Synthetic
AQIGASSGGAQ

SEQ ID NO: 1035 - protein, 11 aa, synthetic construct
  REGION 1..11: note = Synthetic
AQIGGAVTTAQ

SEQ ID NO: 1036 - protein, 11 aa, synthetic construct
  REGION 1..11: note = Synthetic
AQIGGHERPAQ

SEQ ID NO: 1037 - protein, 11 aa, synthetic construct
  REGION 1..11: note = Synthetic
AQIGHVSANAQ

SEQ ID NO: 1038 - protein, 11 aa, synthetic construct
  REGION 1..11: note = Synthetic
AQIGRVTNEAQ

SEQ ID NO: 1039 - protein, 11 aa, synthetic construct
  REGION 1..11: note = Synthetic
AQIGSRASDAQ

SEQ ID NO: 1040 - protein, 11 aa, synthetic construct
  REGION 1..11: note = Synthetic
AQIGSSRLGAQ

SEQ ID NO: 1041 - protein, 11 aa, synthetic construct
  REGION 1..11: note = Synthetic
AQIHGKEREAQ

SEQ ID NO: 1042 - protein, 11 aa, synthetic construct
  REGION 1..11: note = Synthetic
AQIHLSSATAQ

SEQ ID NO: 1043 - protein, 11 aa, synthetic construct
  REGION 1..11: note = Synthetic
AQIHSRGDTAQ

SEQ ID NO: 1044 - protein, 11 aa, synthetic construct
  REGION 1..11: note = Synthetic
AQIHSTNPGAQ

SEQ ID NO: 1045 - protein, 11 aa, synthetic construct
  REGION 1..11: note = Synthetic
AQIISNRAVAQ

SEQ ID NO: 1046 - protein, 11 aa, synthetic construct
  REGION 1..11: note = Synthetic
AQILHLPMNAQ

SEQ ID NO: 1047 - protein, 11 aa, synthetic construct
  REGION 1..11: note = Synthetic
AQILTSTVGAQ

SEQ ID NO: 1048 - protein, 11 aa, synthetic construct
  REGION 1..11: note = Synthetic
AQINSTATGAQ

SEQ ID NO: 1049 - protein, 11 aa, synthetic construct
  REGION 1..11: note = Synthetic
AQIPNFPTLAQ

SEQ ID NO: 1050 - protein, 11 aa, synthetic construct
  REGION 1..11: note = Synthetic
AQIPSGNVSAQ

SEQ ID NO: 1051 - protein, 11 aa, synthetic construct
  REGION 1..11: note = Synthetic
AQIPSKSPAAQ

SEQ ID NO: 1052 - protein, 11 aa, synthetic construct
  REGION 1..11: note = Synthetic
AQIPSTHTGAQ

SEQ ID NO: 1053 - protein, 11 aa, synthetic construct
  REGION 1..11: note = Synthetic
AQIPSTRAGAQ

SEQ ID NO: 1054 - protein, 11 aa, synthetic construct
  REGION 1..11: note = Synthetic
AQIRHGATDAQ

SEQ ID NO: 1055 - protein, 11 aa, synthetic construct
  REGION 1..11: note = Synthetic
AQIRPRPEDAQ

SEQ ID NO: 1056 - protein, 11 aa, synthetic construct
  REGION 1..11: note = Synthetic
AQIRSDHSSAQ

SEQ ID NO: 1057 - protein, 11 aa, synthetic construct
  REGION 1..11: note = Synthetic
AQIRSDSAMAQ

SEQ ID NO: 1058 - protein, 11 aa, synthetic construct
  REGION 1..11: note = Synthetic
AQIRSSTGDAQ

SEQ ID NO: 1059 - protein, 11 aa, synthetic construct
  REGION 1..11: note = Synthetic
AQIRSSTIFAQ

SEQ ID NO: 1060 - protein, 11 aa, synthetic construct
  REGION 1..11: note = Synthetic
AQIRTMSSEAQ

SEQ ID NO: 1061 - protein, 11 aa, synthetic construct
  REGION 1..11: note = Synthetic
AQISNRTSSAQ

SEQ ID NO: 1062 - protein, 11 aa, synthetic construct
  REGION 1..11: note = Synthetic
AQISRQEVLAQ

SEQ ID NO: 1063 - protein, 11 aa, synthetic construct
  REGION 1..11: note = Synthetic
AQISSHSPGAQ

SEQ ID NO: 1064 - protein, 11 aa, synthetic construct
  REGION 1..11: note = Synthetic
AQISSSRLSAQ

SEQ ID NO: 1065 - protein, 11 aa, synthetic construct
  REGION 1..11: note = Synthetic
AQISVSLRSAQ

SEQ ID NO: 1066 - protein, 11 aa, synthetic construct
  REGION 1..11: note = Synthetic
AQITGVPMNAQ

SEQ ID NO: 1067 - protein, 11 aa, synthetic construct
  REGION 1..11: note = Synthetic
AQITRNADNAQ

SEQ ID NO: 1068 - protein, 11 aa, synthetic construct
  REGION 1..11: note = Synthetic
AQITSSTQFAQ

SEQ ID NO: 1069 - protein, 11 aa, synthetic construct
  REGION 1..11: note = Synthetic
AQITTRIEQAQ

SEQ ID NO: 1070 - protein, 11 aa, synthetic construct
  REGION 1..11: note = Synthetic
AQIVKPNITAQ

SEQ ID NO: 1071 - protein, 11 aa, synthetic construct
  REGION 1..11: note = Synthetic
AQIVSRTENAQ

SEQ ID NO: 1072 - protein, 11 aa, synthetic construct
  REGION 1..11: note = Synthetic
AQIVVRGDSAQ

SEQ ID NO: 1073 - protein, 11 aa, synthetic construct
  REGION 1..11: note = Synthetic
AQIYSASTPAQ

SEQ ID NO: 1074 - protein, 11 aa, synthetic construct
  REGION 1..11: note = Synthetic
AQKGQPPFLAQ

SEQ ID NO: 1075 - protein, 11 aa, synthetic construct
  REGION 1..11: note = Synthetic
AQKHHVPFDAQ

SEQ ID NO: 1076 - protein, 11 aa, synthetic construct
  REGION 1..11: note = Synthetic
AQKLDLPMTAQ

SEQ ID NO: 1077 - protein, 11 aa, synthetic construct
  REGION 1..11: note = Synthetic
AQKLLGGLSAQ

SEQ ID NO: 1078 - protein, 11 aa, synthetic construct
  REGION 1..11: note = Synthetic
AQKLSLPTTAQ

SEQ ID NO: 1079 - protein, 11 aa, synthetic construct
  REGION 1..11: note = Synthetic
AQKNGQPVQAQ

SEQ ID NO: 1080 - protein, 11 aa, synthetic construct
  REGION 1..11: note = Synthetic
AQKPYVPHDAQ

SEQ ID NO: 1081 - protein, 11 aa, synthetic construct
  REGION 1..11: note = Synthetic
AQKQRSEDPAQ

SEQ ID NO: 1082 - protein, 11 aa, synthetic construct
  REGION 1..11: note = Synthetic
AQKSGSMLVAQ

SEQ ID NO: 1083 - protein, 11 aa, synthetic construct
  REGION 1..11: note = Synthetic
AQKSQSLAGAQ

SEQ ID NO: 1084 - protein, 11 aa, synthetic construct
  REGION 1..11: note = Synthetic
AQKSQTSYMAQ

SEQ ID NO: 1085 - protein, 11 aa, synthetic construct
  REGION 1..11: note = Synthetic
AQKTTTSLAAQ

SEQ ID NO: 1086 - protein, 11 aa, synthetic construct
  REGION 1..11: note = Synthetic
AQLAGRGAGAQ

SEQ ID NO: 1087 - protein, 11 aa, synthetic construct
  REGION 1..11: note = Synthetic
AQLAPIMREAQ

SEQ ID NO: 1088 - protein, 11 aa, synthetic construct
  REGION 1..11: note = Synthetic
AQLATTRVEAQ

SEQ ID NO: 1089 - protein, 11 aa, synthetic construct
  REGION 1..11: note = Synthetic
AQLDRANPQAQ

SEQ ID NO: 1090 - protein, 11 aa, synthetic construct
  REGION 1..11: note = Synthetic
AQLEHRSGSAQ

SEQ ID NO: 1091 - protein, 11 aa, synthetic construct
  REGION 1..11: note = Synthetic
AQLFGPLLLAQ

SEQ ID NO: 1092 - protein, 11 aa, synthetic construct
  REGION 1..11: note = Synthetic
AQLFSWRNAAQ

SEQ ID NO: 1093 - protein, 11 aa, synthetic construct
  REGION 1..11: note = Synthetic
AQLFTTSNIAQ

SEQ ID NO: 1094 - protein, 11 aa, synthetic construct
  REGION 1..11: note = Synthetic
AQLGHSLTSAQ

SEQ ID NO: 1095 - protein, 11 aa, synthetic construct
  REGION 1..11: note = Synthetic
AQLGHSSQIAQ

SEQ ID NO: 1096 - protein, 11 aa, synthetic construct
  REGION 1..11: note = Synthetic
AQLGNLGHQAQ

SEQ ID NO: 1097 - protein, 11 aa, synthetic construct
  REGION 1..11: note = Synthetic
AQLGRVQSDAQ

SEQ ID NO: 1098 - protein, 11 aa, synthetic construct
  REGION 1..11: note = Synthetic
AQLGSSSVSAQ

SEQ ID NO: 1099 - protein, 11 aa, synthetic construct
  REGION 1..11: note = Synthetic
AQLHGPTLLAQ

SEQ ID NO: 1100 - protein, 11 aa, synthetic construct
  REGION 1..11: note = Synthetic
AQLHGVPMNAQ

SEQ ID NO: 1101 - protein, 11 aa, synthetic construct
  REGION 1..11: note = Synthetic
AQLHHNRESAQ

SEQ ID NO: 1102 - protein, 11 aa, synthetic construct
  REGION 1..11: note = Synthetic
AQLHNFPDNAQ

SEQ ID NO: 1103 - protein, 11 aa, synthetic construct
  REGION 1..11: note = Synthetic
AQLHSIGTSAQ

SEQ ID NO: 1104 - protein, 11 aa, synthetic construct
  REGION 1..11: note = Synthetic
AQLHSSTLGAQ

SEQ ID NO: 1105 - protein, 11 aa, synthetic construct
  REGION 1..11: note = Synthetic
AQLIHNGAAAQ

SEQ ID NO: 1106 - protein, 11 aa, synthetic construct
  REGION 1..11: note = Synthetic
AQLKVGPMDAQ

SEQ ID NO: 1107 - protein, 11 aa, synthetic construct
  REGION 1..11: note = Synthetic
AQLKVPVLMAQ

SEQ ID NO: 1108 - protein, 11 aa, synthetic construct
  REGION 1..11: note = Synthetic
AQLLDHISKAQ

SEQ ID NO: 1109 - protein, 11 aa, synthetic construct
  REGION 1..11: note = Synthetic
AQLLPATRDAQ

SEQ ID NO: 1110 - protein, 11 aa, synthetic construct
  REGION 1..11: note = Synthetic
AQLNHGANAAQ

SEQ ID NO: 1111 - protein, 11 aa, synthetic construct
  REGION 1..11: note = Synthetic
AQLNHTRPGAQ

SEQ ID NO: 1112 - protein, 11 aa, synthetic construct
  REGION 1..11: note = Synthetic
AQLNSPSFLAQ

SEQ ID NO: 1113 - protein, 11 aa, synthetic construct
  REGION 1..11: note = Synthetic
AQLNSSRASAQ

SEQ ID NO: 1114 - protein, 11 aa, synthetic construct
  REGION 1..11: note = Synthetic
AQLNSVKHYAQ

SEQ ID NO: 1115 - protein, 11 aa, synthetic construct
  REGION 1..11: note = Synthetic
AQLNTTKTNAQ

SEQ ID NO: 1116 - protein, 11 aa, synthetic construct
  REGION 1..11: note = Synthetic
AQLPGRSTSAQ

SEQ ID NO: 1117 - protein, 11 aa, synthetic construct
  REGION 1..11: note = Synthetic
AQLPSYHSAAQ

SEQ ID NO: 1118 - protein, 11 aa, synthetic construct
  REGION 1..11: note = Synthetic
AQLQRSTDGAQ

SEQ ID NO: 1119 - protein, 11 aa, synthetic construct
  REGION 1..11: note = Synthetic
AQLQTTYSSAQ

SEQ ID NO: 1120 - protein, 11 aa, synthetic construct
  REGION 1..11: note = Synthetic
AQLRNSSGDAQ

SEQ ID NO: 1121 - protein, 11 aa, synthetic construct
  REGION 1..11: note = Synthetic
AQLRSLSAEAQ

SEQ ID NO: 1122 - protein, 11 aa, synthetic construct
  REGION 1..11: note = Synthetic
AQLSHSYSSAQ

SEQ ID NO: 1123 - protein, 11 aa, synthetic construct
  REGION 1..11: note = Synthetic
AQLSNSVKTAQ

SEQ ID NO: 1124 - protein, 11 aa, synthetic construct
  REGION 1..11: note = Synthetic
AQLSSHAYQAQ

SEQ ID NO: 1125 - protein, 11 aa, synthetic construct
  REGION 1..11: note = Synthetic
AQLSSRPDAAQ

SEQ ID NO: 1126 - protein, 11 aa, synthetic construct
  REGION 1..11: note = Synthetic
AQLSSSRSAAQ

SEQ ID NO: 1127 - protein, 11 aa, synthetic construct
  REGION 1..11: note = Synthetic
AQLSSSYTVAQ

SEQ ID NO: 1128 - protein, 11 aa, synthetic construct
  REGION 1..11: note = Synthetic
AQLSTSYSSAQ

SEQ ID NO: 1129 - protein, 11 aa, synthetic construct
  REGION 1..11: note = Synthetic
AQLTHVSNIAQ

SEQ ID NO: 1130 - protein, 11 aa, synthetic construct
  REGION 1..11: note = Synthetic
AQLTTSVRIAQ

SEQ ID NO: 1131 - protein, 11 aa, synthetic construct
  REGION 1..11: note = Synthetic
AQLTTTRTEAQ

SEQ ID NO: 1132 - protein, 11 aa, synthetic construct
  REGION 1..11: note = Synthetic
AQLTVAGRSAQ

SEQ ID NO: 1133 - protein, 11 aa, synthetic construct
  REGION 1..11: note = Synthetic
AQMGSKSNEAQ

SEQ ID NO: 1134 - protein, 11 aa, synthetic construct
  REGION 1..11: note = Synthetic
AQMKVPILLAQ

SEQ ID NO: 1135 - protein, 11 aa, synthetic construct
  REGION 1..11: note = Synthetic
AQMLAGRESAQ

SEQ ID NO: 1136 - protein, 11 aa, synthetic construct
  REGION 1..11: note = Synthetic
AQMNAGRSGAQ

SEQ ID NO: 1137 - protein, 11 aa, synthetic construct
  REGION 1..11: note = Synthetic
AQMNSSKPTAQ

SEQ ID NO: 1138 - protein, 11 aa, synthetic construct
  REGION 1..11: note = Synthetic
AQMNSVRTGAQ

SEQ ID NO: 1139 - protein, 11 aa, synthetic construct
  REGION 1..11: note = Synthetic
AQMNTSRSPAQ

SEQ ID NO: 1140 - protein, 11 aa, synthetic construct
  REGION 1..11: note = Synthetic
AQMNTTRVPAQ

SEQ ID NO: 1141 - protein, 11 aa, synthetic construct
  REGION 1..11: note = Synthetic
AQMPTKMTDAQ

SEQ ID NO: 1142 - protein, 11 aa, synthetic construct
  REGION 1..11: note = Synthetic
AQMRTGSNEAQ

SEQ ID NO: 1143 - protein, 11 aa, synthetic construct
  REGION 1..11: note = Synthetic
AQMRTLTGDAQ

SEQ ID NO: 1144 - protein, 11 aa, synthetic construct
  REGION 1..11: note = Synthetic
AQMRTSHTEAQ

SEQ ID NO: 1145 - protein, 11 aa, synthetic construct
  REGION 1..11: note = Synthetic
AQMRTSNLTAQ

SEQ ID NO: 1146 - protein, 11 aa, synthetic construct
  REGION 1..11: note = Synthetic
AQMSGHRQDAQ

SEQ ID NO: 1147 - protein, 11 aa, synthetic construct
  REGION 1..11: note = Synthetic
AQMSHSSGAAQ

SEQ ID NO: 1148 - protein, 11 aa, synthetic construct
  REGION 1..11: note = Synthetic
AQMSNRVLEAQ

SEQ ID NO: 1149 - protein, 11 aa, synthetic construct
  REGION 1..11: note = Synthetic
AQMSTTRSSAQ

SEQ ID NO: 1150 - protein, 11 aa, synthetic construct
  REGION 1..11: note = Synthetic
AQMTSKPESAQ

SEQ ID NO: 1151 - protein, 11 aa, synthetic construct
  REGION 1..11: note = Synthetic
AQMVNRVQSAQ

SEQ ID NO: 1152 - protein, 11 aa, synthetic construct
  REGION 1..11: note = Synthetic
AQMVRSNDPAQ

SEQ ID NO: 1153 - protein, 11 aa, synthetic construct
  REGION 1..11: note = Synthetic
AQMYTTGQSAQ

SEQ ID NO: 1154 - protein, 11 aa, synthetic construct
  REGION 1..11: note = Synthetic
AQNAGRYGDAQ

SEQ ID NO: 1155 - protein, 11 aa, synthetic construct
  REGION 1..11: note = Synthetic
AQNGARTPPAQ

SEQ ID NO: 1156 - protein, 11 aa, synthetic construct
  REGION 1..11: note = Synthetic
AQNGQRLSPAQ

SEQ ID NO: 1157 - protein, 11 aa, synthetic construct
  REGION 1..11: note = Synthetic
AQNGRVGSNAQ

SEQ ID NO: 1158 - protein, 11 aa, synthetic construct
  REGION 1..11: note = Synthetic
AQNGSATRVAQ

SEQ ID NO: 1159 - protein, 11 aa, synthetic construct
  REGION 1..11: note = Synthetic
AQNGSKLAGAQ

SEQ ID NO: 1160 - protein, 11 aa, synthetic construct
  REGION 1..11: note = Synthetic
AQNGSRVIAAQ

SEQ ID NO: 1161 - protein, 11 aa, synthetic construct
  REGION 1..11: note = Synthetic
AQNGTMRLAAQ

SEQ ID NO: 1162 - protein, 11 aa, synthetic construct
  REGION 1..11: note = Synthetic
AQNHRPSGDAQ

SEQ ID NO: 1163 - protein, 11 aa, synthetic construct
  REGION 1..11: note = Synthetic
AQNLANTRDAQ

SEQ ID NO: 1164 - protein, 11 aa, synthetic construct
  REGION 1..11: note = Synthetic
AQNLQPGKIAQ

SEQ ID NO: 1165 - protein, 11 aa, synthetic construct
  REGION 1..11: note = Synthetic
AQNLRGTSVAQ

SEQ ID NO: 1166 - protein, 11 aa, synthetic construct
  REGION 1..11: note = Synthetic
AQNLSSVNGAQ

SEQ ID NO: 1167 - protein, 11 aa, synthetic construct
  REGION 1..11: note = Synthetic
AQNRTGGFDAQ

SEQ ID NO: 1168 - protein, 11 aa, synthetic construct
  REGION 1..11: note = Synthetic
AQNRVAQGEAQ

SEQ ID NO: 1169 - protein, 11 aa, synthetic construct
  REGION 1..11: note = Synthetic
AQNSATMRQAQ

SEQ ID NO: 1170 - protein, 11 aa, synthetic construct
  REGION 1..11: note = Synthetic
AQNSDPYRFAQ

SEQ ID NO: 1171 - protein, 11 aa, synthetic construct
  REGION 1..11: note = Synthetic
AQNSHISRDAQ

SEQ ID NO: 1172 - protein, 11 aa, synthetic construct
  REGION 1..11: note = Synthetic
AQNSQTLRSAQ

SEQ ID NO: 1173 - protein, 11 aa, synthetic construct
  REGION 1..11: note = Synthetic
AQNSRAAEHAQ

SEQ ID NO: 1174 - protein, 11 aa, synthetic construct
  REGION 1..11: note = Synthetic
AQNSRGPVAAQ

SEQ ID NO: 1175 - protein, 11 aa, synthetic construct
  REGION 1..11: note = Synthetic
AQNSSHANSAQ

SEQ ID NO: 1176 - protein, 11 aa, synthetic construct
  REGION 1..11: note = Synthetic
AQNSTQYNAAQ

SEQ ID NO: 1177 - protein, 11 aa, synthetic construct
  REGION 1..11: note = Synthetic
AQNTGHASMAQ

SEQ ID NO: 1178 - protein, 11 aa, synthetic construct
  REGION 1..11: note = Synthetic
AQNTGRSDSAQ

SEQ ID NO: 1179 - protein, 11 aa, synthetic construct
  REGION 1..11: note = Synthetic
AQNTNMPQYAQ

SEQ ID NO: 1180 - protein, 11 aa, synthetic construct
  REGION 1..11: note = Synthetic
AQNTSSVRGAQ

SEQ ID NO: 1181 - protein, 11 aa, synthetic construct
  REGION 1..11: note = Synthetic
AQNTVNVSGAQ

SEQ ID NO: 1182 - protein, 11 aa, synthetic construct
  REGION 1..11: note = Synthetic
AQNVAGRLDAQ

SEQ ID NO: 1183 - protein, 11 aa, synthetic construct
  REGION 1..11: note = Synthetic
AQNVSNLHGAQ

SEQ ID NO: 1184 - protein, 11 aa, synthetic construct
  REGION 1..11: note = Synthetic
AQNVSQLRDAQ

SEQ ID NO: 1185 - protein, 11 aa, synthetic construct
  REGION 1..11: note = Synthetic
AQNVTRGHEAQ

SEQ ID NO: 1186 - protein, 11 aa, synthetic construct
  REGION 1..11: note = Synthetic
AQNVYNVSGAQ

SEQ ID NO: 1187 - protein, 11 aa, synthetic construct
  REGION 1..11: note = Synthetic
AQPARPNNPAQ

SEQ ID NO: 1188 - protein, 11 aa, synthetic construct
  REGION 1..11: note = Synthetic
AQPFTPKPMAQ

SEQ ID NO: 1189 - protein, 11 aa, synthetic construct
  REGION 1..11: note = Synthetic
AQPGVGKQPAQ

SEQ ID NO: 1190 - protein, 11 aa, synthetic construct
  REGION 1..11: note = Synthetic
AQPHDSVMWAQ

SEQ ID NO: 1191 - protein, 11 aa, synthetic construct
  REGION 1..11: note = Synthetic
AQPINLPMSAQ

SEQ ID NO: 1192 - protein, 11 aa, synthetic construct
  REGION 1..11: note = Synthetic
AQPKSSHEWAQ

SEQ ID NO: 1193 - protein, 11 aa, synthetic construct
  REGION 1..11: note = Synthetic
AQPNNTIRGAQ

SEQ ID NO: 1194 - protein, 11 aa, synthetic construct
  REGION 1..11: note = Synthetic
AQPQKTGSAAQ

SEQ ID NO: 1195 - protein, 11 aa, synthetic construct
  REGION 1..11: note = Synthetic
AQPQSGPLPAQ

SEQ ID NO: 1196 - protein, 11 aa, synthetic construct
  REGION 1..11: note = Synthetic
AQPQSNRPAAQ

SEQ ID NO: 1197 - protein, 11 aa, synthetic construct
  REGION 1..11: note = Synthetic
AQPQSRGDSAQ

SEQ ID NO: 1198 - protein, 11 aa, synthetic construct
  REGION 1..11: note = Synthetic
AQPRLPEVFAQ

SEQ ID NO: 1199 - protein, 11 aa, synthetic construct
  REGION 1..11: note = Synthetic
AQPRTGTPPAQ

SEQ ID NO: 1200 - protein, 11 aa, synthetic construct
  REGION 1..11: note = Synthetic
AQPSHSVLFAQ

SEQ ID NO: 1201 - protein, 11 aa, synthetic construct
  REGION 1..11: note = Synthetic
AQPSRGQTAAQ

SEQ ID NO: 1202 - protein, 11 aa, synthetic construct
  REGION 1..11: note = Synthetic
AQPSRGQTTAQ

SEQ ID NO: 1203 - protein, 11 aa, synthetic construct
  REGION 1..11: note = Synthetic
AQPSTDTFWAQ

SEQ ID NO: 1204 - protein, 11 aa, synthetic construct
  REGION 1..11: note = Synthetic
AQPSVTGRSAQ

SEQ ID NO: 1205 - protein, 11 aa, synthetic construct
  REGION 1..11: note = Synthetic
AQPSYGGSGAQ

SEQ ID NO: 1206 - protein, 11 aa, synthetic construct
  REGION 1..11: note = Synthetic
AQPTGLPINAQ

SEQ ID NO: 1207 - protein, 11 aa, synthetic construct
  REGION 1..11: note = Synthetic
AQPTQRTGTAQ

SEQ ID NO: 1208 - protein, 11 aa, synthetic construct
  REGION 1..11: note = Synthetic
AQPTRGTGTAQ

SEQ ID NO: 1209 - protein, 11 aa, synthetic construct
  REGION 1..11: note = Synthetic
AQPTSALPMAQ

SEQ ID NO: 1210 - protein, 11 aa, synthetic construct
  REGION 1..11: note = Synthetic
AQPVSRSPAAQ

SEQ ID NO: 1211 - protein, 11 aa, synthetic construct
  REGION 1..11: note = Synthetic
AQQAGRSVAAQ

SEQ ID NO: 1212 - protein, 11 aa, synthetic construct
  REGION 1..11: note = Synthetic
AQQAGSSVAAQ

SEQ ID NO: 1213 - protein, 11 aa, synthetic construct
  REGION 1..11: note = Synthetic
AQQASRNEIAQ

SEQ ID NO: 1214 - protein, 11 aa, synthetic construct
  REGION 1..11: note = Synthetic
AQQATRGDSAQ

SEQ ID NO: 1215 - protein, 11 aa, synthetic construct
  REGION 1..11: note = Synthetic
AQQDRILSAAQ

SEQ ID NO: 1216 - protein, 11 aa, synthetic construct
  REGION 1..11: note = Synthetic
AQQDSMRGLAQ

SEQ ID NO: 1217 - protein, 11 aa, synthetic construct
  REGION 1..11: note = Synthetic
AQQDTRSHAAQ

SEQ ID NO: 1218 - protein, 11 aa, synthetic construct
  REGION 1..11: note = Synthetic
AQQGGLILLAQ

SEQ ID NO: 1219 - protein, 11 aa, synthetic construct
  REGION 1..11: note = Synthetic
AQQGSIRSDAQ

SEQ ID NO: 1220 - protein, 11 aa, synthetic construct
  REGION 1..11: note = Synthetic
AQQGYGGQTAQ

SEQ ID NO: 1221 - protein, 11 aa, synthetic construct
  REGION 1..11: note = Synthetic
AQQHAPSLLAQ

SEQ ID NO: 1222 - protein, 11 aa, synthetic construct
  REGION 1..11: note = Synthetic
AQQLPGGLHAQ

SEQ ID NO: 1223 - protein, 11 aa, synthetic construct
  REGION 1..11: note = Synthetic
AQQLSNKTNAQ

SEQ ID NO: 1224 - protein, 11 aa, synthetic construct
  REGION 1..11: note = Synthetic
AQQLSRDVPAQ

SEQ ID NO: 1225 - protein, 11 aa, synthetic construct
  REGION 1..11: note = Synthetic
AQQLVQHRDAQ

SEQ ID NO: 1226 - protein, 11 aa, synthetic construct
  REGION 1..11: note = Synthetic
AQQMRTGGIAQ

SEQ ID NO: 1227 - protein, 11 aa, synthetic construct
  REGION 1..11: note = Synthetic
AQQPGYSSDAQ

SEQ ID NO: 1228 - protein, 11 aa, synthetic construct
  REGION 1..11: note = Synthetic
AQQPRANQIAQ

SEQ ID NO: 1229 - protein, 11 aa, synthetic construct
  REGION 1..11: note = Synthetic
AQQPRTNESAQ

SEQ ID NO: 1230 - protein, 11 aa, synthetic construct
  REGION 1..11: note = Synthetic
AQQQLPVLMAQ

SEQ ID NO: 1231 - protein, 11 aa, synthetic construct
  REGION 1..11: note = Synthetic
AQQRESPLDAQ

SEQ ID NO: 1232 - protein, 11 aa, synthetic construct
  REGION 1..11: note = Synthetic
AQQRHLPTDAQ

SEQ ID NO: 1233 - protein, 11 aa, synthetic construct
  REGION 1..11: note = Synthetic
AQQSAGKLGAQ

SEQ ID NO: 1234 - protein, 11 aa, synthetic construct
  REGION 1..11: note = Synthetic
AQQSLSSLSAQ

SEQ ID NO: 1235 - protein, 11 aa, synthetic construct
  REGION 1..11: note = Synthetic
AQQSNLPMTAQ

SEQ ID NO: 1236 - protein, 11 aa, synthetic construct
  REGION 1..11: note = Synthetic
AQQSNVYREAQ

SEQ ID NO: 1237 - protein, 11 aa, synthetic construct
  REGION 1..11: note = Synthetic
AQQTSRLQEAQ

SEQ ID NO: 1238 - protein, 11 aa, synthetic construct
  REGION 1..11: note = Synthetic
AQQTTDRSGAQ

SEQ ID NO: 1239 - protein, 11 aa, synthetic construct
  REGION 1..11: note = Synthetic
AQQVGPILMAQ

SEQ ID NO: 1240 - protein, 11 aa, synthetic construct
  REGION 1..11: note = Synthetic
AQQVGSYQHAQ

SEQ ID NO: 1241 - protein, 11 aa, synthetic construct
  REGION 1..11: note = Synthetic
AQQVSTARPAQ

SEQ ID NO: 1242 - protein, 11 aa, synthetic construct
  REGION 1..11: note = Synthetic
AQQYGSVSQAQ

SEQ ID NO: 1243 - protein, 11 aa, synthetic construct
  REGION 1..11: note = Synthetic
AQQYSSGSSAQ

SEQ ID NO: 1244 - protein, 11 aa, synthetic construct
  REGION 1..11: note = Synthetic
AQRAGVSQIAQ

SEQ ID NO: 1245 - protein, 11 aa, synthetic construct
  REGION 1..11: note = Synthetic
AQRDTTYTTAQ

SEQ ID NO: 1246 - protein, 11 aa, synthetic construct
  REGION 1..11: note = Synthetic
AQRERSSEVAQ

SEQ ID NO: 1247 - protein, 11 aa, synthetic construct
  REGION 1..11: note = Synthetic
AQRGHGSSDAQ

SEQ ID NO: 1248 - protein, 11 aa, synthetic construct
  REGION 1..11: note = Synthetic
AQRGPLTLLAQ

SEQ ID NO: 1249 - protein, 11 aa, synthetic construct
  REGION 1..11: note = Synthetic
AQRGTLDLLAQ

SEQ ID NO: 1250 - protein, 11 aa, synthetic construct
  REGION 1..11: note = Synthetic
AQRIASPYDAQ

SEQ ID NO: 1251 - protein, 11 aa, synthetic construct
  REGION 1..11: note = Synthetic
AQRIGTATVAQ

SEQ ID NO: 1252 - protein, 11 aa, synthetic construct
  REGION 1..11: note = Synthetic
AQRIGTMSAAQ

SEQ ID NO: 1253 - protein, 11 aa, synthetic construct
  REGION 1..11: note = Synthetic
AQRLPGGLSAQ

SEQ ID NO: 1254 - protein, 11 aa, synthetic construct
  REGION 1..11: note = Synthetic
AQRLTQHEPAQ

SEQ ID NO: 1255 - protein, 11 aa, synthetic construct
  REGION 1..11: note = Synthetic
AQRMSNGSTAQ

SEQ ID NO: 1256 - protein, 11 aa, synthetic construct
  REGION 1..11: note = Synthetic
AQRMSSSSDAQ

SEQ ID NO: 1257 - protein, 11 aa, synthetic construct
  REGION 1..11: note = Synthetic
AQRNHSAGDAQ

SEQ ID NO: 1258 - protein, 11 aa, synthetic construct
  REGION 1..11: note = Synthetic
AQRNLPQVYAQ

SEQ ID NO: 1259 - protein, 11 aa, synthetic construct
  REGION 1..11: note = Synthetic
AQRNYSPTEAQ

SEQ ID NO: 1260 - protein, 11 aa, synthetic construct
  REGION 1..11: note = Synthetic
AQRPEVPNWAQ

SEQ ID NO: 1261 - protein, 11 aa, synthetic construct
  REGION 1..11: note = Synthetic
AQRPQGSAGAQ

SEQ ID NO: 1262 - protein, 11 aa, synthetic construct
  REGION 1..11: note = Synthetic
AQRPTMPEYAQ

SEQ ID NO: 1263 - protein, 11 aa, synthetic construct
  REGION 1..11: note = Synthetic
AQRSDRLTDAQ

SEQ ID NO: 1264 - protein, 11 aa, synthetic construct
  REGION 1..11: note = Synthetic
AQRSGMAVEAQ

SEQ ID NO: 1265 - protein, 11 aa, synthetic construct
  REGION 1..11: note = Synthetic
AQRSGSGSDAQ

SEQ ID NO: 1266 - protein, 11 aa, synthetic construct
  REGION 1..11: note = Synthetic
AQRSGSSADAQ

SEQ ID NO: 1267 - protein, 11 aa, synthetic construct
  REGION 1..11: note = Synthetic
AQRSNLLAEAQ

SEQ ID NO: 1268 - protein, 11 aa, synthetic construct
  REGION 1..11: note = Synthetic
AQRSNSSVGAQ

SEQ ID NO: 1269 - protein, 11 aa, synthetic construct
  REGION 1..11: note = Synthetic
AQRSTAIVAAQ

SEQ ID NO: 1270 - protein, 11 aa, synthetic construct
  REGION 1..11: note = Synthetic
AQRTGMTGAAQ

SEQ ID NO: 1271 - protein, 11 aa, synthetic construct
  REGION 1..11: note = Synthetic
AQRTGSVVNAQ

SEQ ID NO: 1272 - protein, 11 aa, synthetic construct
  REGION 1..11: note = Synthetic
AQRTGTQSSAQ

SEQ ID NO: 1273 - protein, 11 aa, synthetic construct
  REGION 1..11: note = Synthetic
AQRTIGTSTAQ

SEQ ID NO: 1274 - protein, 11 aa, synthetic construct
  REGION 1..11: note = Synthetic
AQRTSQIQSAQ

SEQ ID NO: 1275 - protein, 11 aa, synthetic construct
  REGION 1..11: note = Synthetic
AQRVGASGTAQ

SEQ ID NO: 1276 - protein, 11 aa, synthetic construct
  REGION 1..11: note = Synthetic
AQRVGSSLSAQ

SEQ ID NO: 1277 - protein, 11 aa, synthetic construct
  REGION 1..11: note = Synthetic
AQRVPAGGGAQ

SEQ ID NO: 1278 - protein, 11 aa, synthetic construct
  REGION 1..11: note = Synthetic
AQRVSATQTAQ

SEQ ID NO: 1279 - protein, 11 aa, synthetic construct
  REGION 1..11: note = Synthetic
AQRVVGGLTAQ

SEQ ID NO: 1280 - protein, 11 aa, synthetic construct
  REGION 1..11: note = Synthetic
AQSALHVSGAQ

SEQ ID NO: 1281 - protein, 11 aa, synthetic construct
  REGION 1..11: note = Synthetic
AQSASLGGYAQ

SEQ ID NO: 1282 - protein, 11 aa, synthetic construct
  REGION 1..11: note = Synthetic
AQSASLRSDAQ

SEQ ID NO: 1283 - protein, 11 aa, synthetic construct
  REGION 1..11: note = Synthetic
AQSAVVRSGAQ

SEQ ID NO: 1284 - protein, 11 aa, synthetic construct
  REGION 1..11: note = Synthetic
AQSDMPRTFAQ

SEQ ID NO: 1285 - protein, 11 aa, synthetic construct
  REGION 1..11: note = Synthetic
AQSDTRSMSAQ

SEQ ID NO: 1286 - protein, 11 aa, synthetic construct
  REGION 1..11: note = Synthetic
AQSFNHGATAQ

SEQ ID NO: 1287 - protein, 11 aa, synthetic construct
  REGION 1..11: note = Synthetic
AQSFTRIPPAQ

SEQ ID NO: 1288 - protein, 11 aa, synthetic construct
  REGION 1..11: note = Synthetic
AQSGNFSGGAQ

SEQ ID NO: 1289 - protein, 11 aa, synthetic construct
  REGION 1..11: note = Synthetic
AQSGSALHGAQ

SEQ ID NO: 1290 - protein, 11 aa, synthetic construct
  REGION 1..11: note = Synthetic
AQSGSRLLDAQ

SEQ ID NO: 1291 - protein, 11 aa, synthetic construct
  REGION 1..11: note = Synthetic
AQSGSSRDMAQ

SEQ ID NO: 1292 - protein, 11 aa, synthetic construct
  REGION 1..11: note = Synthetic
AQSGSSRPSAQ

SEQ ID NO: 1293 - protein, 11 aa, synthetic construct
  REGION 1..11: note = Synthetic
AQSGSYSTVAQ

SEQ ID NO: 1294 - protein, 11 aa, synthetic construct
  REGION 1..11: note = Synthetic
AQSHEKFLYAQ

SEQ ID NO: 1295 - protein, 11 aa, synthetic construct
  REGION 1..11: note = Synthetic
AQSHSASREAQ

SEQ ID NO: 1296 - protein, 11 aa, synthetic construct
  REGION 1..11: note = Synthetic
AQSHSNTGNAQ

SEQ ID NO: 1297 - protein, 11 aa, synthetic construct
  REGION 1..11: note = Synthetic
AQSIPGKGMAQ

SEQ ID NO: 1298 - protein, 11 aa, synthetic construct
  REGION 1..11: note = Synthetic
AQSIRSQLPAQ

SEQ ID NO: 1299 - protein, 11 aa, synthetic construct
  REGION 1..11: note = Synthetic
AQSISAGRVAQ

SEQ ID NO: 1300 - protein, 11 aa, synthetic construct
  REGION 1..11: note = Synthetic
AQSISGRSDAQ

SEQ ID NO: 1301 - protein, 11 aa, synthetic construct
  REGION 1..11: note = Synthetic
AQSISGRVGAQ

SEQ ID NO: 1302 - protein, 11 aa, synthetic construct
  REGION 1..11: note = Synthetic
AQSISRQSEAQ

SEQ ID NO: 1303 - protein, 11 aa, synthetic construct
  REGION 1..11: note = Synthetic
AQSISRSGYAQ

SEQ ID NO: 1304 - protein, 11 aa, synthetic construct
  REGION 1..11: note = Synthetic
AQSLAAEFWAQ

SEQ ID NO: 1305 - protein, 11 aa, synthetic construct
  REGION 1..11: note = Synthetic
AQSLDPLGLAQ

SEQ ID NO: 1306 - protein, 11 aa, synthetic construct
  REGION 1..11: note = Synthetic
AQSLIHSIWAQ

SEQ ID NO: 1307 - protein, 11 aa, synthetic construct
  REGION 1..11: note = Synthetic
AQSLNRVVEAQ

SEQ ID NO: 1308 - protein, 11 aa, synthetic construct
  REGION 1..11: note = Synthetic
AQSLQRSVDAQ

SEQ ID NO: 1309 - protein, 11 aa, synthetic construct
  REGION 1..11: note = Synthetic
AQSLSRDGTAQ

SEQ ID NO: 1310 - protein, 11 aa, synthetic construct
  REGION 1..11: note = Synthetic
AQSLVKSGQAQ

SEQ ID NO: 1311 - protein, 11 aa, synthetic construct
  REGION 1..11: note = Synthetic
AQSMSRLNEAQ

SEQ ID NO: 1312 - protein, 11 aa, synthetic construct
  REGION 1..11: note = Synthetic
AQSNTLRNVAQ

SEQ ID NO: 1313 - protein, 11 aa, synthetic construct
  REGION 1..11: note = Synthetic
AQSNYVRSPAQ

SEQ ID NO: 1314 - protein, 11 aa, synthetic construct
  REGION 1..11: note = Synthetic
AQSPRSNVTAQ

SEQ ID NO: 1315 - protein, 11 aa, synthetic construct
  REGION 1..11: note = Synthetic
AQSPRSSIGAQ

SEQ ID NO: 1316 - protein, 11 aa, synthetic construct
  REGION 1..11: note = Synthetic
AQSPVAPNWAQ

SEQ ID NO: 1317 - protein, 11 aa, synthetic construct
  REGION 1..11: note = Synthetic
AQSQALRADAQ

SEQ ID NO: 1318 - protein, 11 aa, synthetic construct
  REGION 1..11: note = Synthetic
AQSQGVRVPAQ

SEQ ID NO: 1319 - protein, 11 aa, synthetic construct
  REGION 1..11: note = Synthetic
AQSQKMLHGAQ

SEQ ID NO: 1320 - protein, 11 aa, synthetic construct
  REGION 1..11: note = Synthetic
AQSQPGPFNAQ

SEQ ID NO: 1321 - protein, 11 aa, synthetic construct
  REGION 1..11: note = Synthetic
AQSQSAKGPAQ

SEQ ID NO: 1322 - protein, 11 aa, synthetic construct
  REGION 1..11: note = Synthetic
AQSRGQTTDAQ

SEQ ID NO: 1323 - protein, 11 aa, synthetic construct
  REGION 1..11: note = Synthetic
AQSRGSETTAQ

SEQ ID NO: 1324 - protein, 11 aa, synthetic construct
  REGION 1..11: note = Synthetic
AQSRISNVSAQ

SEQ ID NO: 1325 - protein, 11 aa, synthetic construct
  REGION 1..11: note = Synthetic
AQSRQTGLTAQ

SEQ ID NO: 1326 - protein, 11 aa, synthetic construct
  REGION 1..11: note = Synthetic
AQSRSQATDAQ

SEQ ID NO: 1327 - protein, 11 aa, synthetic construct
  REGION 1..11: note = Synthetic
AQSRSTDTGAQ

SEQ ID NO: 1328 - protein, 11 aa, synthetic construct
  REGION 1..11: note = Synthetic
AQSRVGLSTAQ

SEQ ID NO: 1329 - protein, 11 aa, synthetic construct
  REGION 1..11: note = Synthetic
AQSSAGKSPAQ

SEQ ID NO: 1330 - protein, 11 aa, synthetic construct
  REGION 1..11: note = Synthetic
AQSSAGRTGAQ

SEQ ID NO: 1331 - protein, 11 aa, synthetic construct
  REGION 1..11: note = Synthetic
AQSSATVREAQ

SEQ ID NO: 1332 - protein, 11 aa, synthetic construct
  REGION 1..11: note = Synthetic
AQSSDSRSSAQ

SEQ ID NO: 1333 - protein, 11 aa, synthetic construct
  REGION 1..11: note = Synthetic
AQSSGISRDAQ

SEQ ID NO: 1334 - protein, 11 aa, synthetic construct
  REGION 1..11: note = Synthetic
AQSSGPIILAQ

SEQ ID NO: 1335 - protein, 11 aa, synthetic construct
  REGION 1..11: note = Synthetic
AQSSHSANMAQ

SEQ ID NO: 1336 - protein, 11 aa, synthetic construct
  REGION 1..11: note = Synthetic
AQSSIVRQEAQ

SEQ ID NO: 1337 - protein, 11 aa, synthetic construct
  REGION 1..11: note = Synthetic
AQSSQNVSGAQ

SEQ ID NO: 1338 - protein, 11 aa, synthetic construct
  REGION 1..11: note = Synthetic
AQSSQRGHEAQ

SEQ ID NO: 1339 - protein, 11 aa, synthetic construct
  REGION 1..11: note = Synthetic
AQSSSSRGPAQ

SEQ ID NO: 1340 - protein, 11 aa, synthetic construct
  REGION 1..11: note = Synthetic
AQSSTVRNAAQ

SEQ ID NO: 1341 - protein, 11 aa, synthetic construct
  REGION 1..11: note = Synthetic
AQSSVTHSSAQ

SEQ ID NO: 1342 - protein, 11 aa, synthetic construct
  REGION 1..11: note = Synthetic
AQSSYQSNGAQ

SEQ ID NO: 1343 - protein, 11 aa, synthetic construct
  REGION 1..11: note = Synthetic
AQSTAARPVAQ

SEQ ID NO: 1344 - protein, 11 aa, synthetic construct
  REGION 1..11: note = Synthetic
AQSTGHNTQAQ

SEQ ID NO: 1345 - protein, 11 aa, synthetic construct
  REGION 1..11: note = Synthetic
AQSTGSSTSAQ

SEQ ID NO: 1346 - protein, 11 aa, synthetic construct
  REGION 1..11: note = Synthetic
AQSTGTPNWAQ

SEQ ID NO: 1347 - protein, 11 aa, synthetic construct
  REGION 1..11: note = Synthetic
AQSTHIARDAQ

SEQ ID NO: 1348 - protein, 11 aa, synthetic construct
  REGION 1..11: note = Synthetic
AQSTIVYREAQ

SEQ ID NO: 1349 - protein, 11 aa, synthetic construct
  REGION 1..11: note = Synthetic
AQSTMQNRIAQ

SEQ ID NO: 1350 - protein, 11 aa, synthetic construct
  REGION 1..11: note = Synthetic
AQSTNHGNNAQ

SEQ ID NO: 1351 - protein, 11 aa, synthetic construct
  REGION 1..11: note = Synthetic
AQSTQIHAGAQ

SEQ ID NO: 1352 - protein, 11 aa, synthetic construct
  REGION 1..11: note = Synthetic
AQSTQLRSGAQ

SEQ ID NO: 1353 - protein, 11 aa, synthetic construct
  REGION 1..11: note = Synthetic
AQSTRALQPAQ

SEQ ID NO: 1354 - protein, 11 aa, synthetic construct
  REGION 1..11: note = Synthetic
AQSTRSSDSAQ

SEQ ID NO: 1355 - protein, 11 aa, synthetic construct
  REGION 1..11: note = Synthetic
AQSTRTAPEAQ

SEQ ID NO: 1356 - protein, 11 aa, synthetic construct
  REGION 1..11: note = Synthetic
AQSTSNRPAAQ

SEQ ID NO: 1357 - protein, 11 aa, synthetic construct
  REGION 1..11: note = Synthetic
AQSTSPPRPAQ

SEQ ID NO: 1358 - protein, 11 aa, synthetic construct
  REGION 1..11: note = Synthetic
AQSTSRLPAAQ

SEQ ID NO: 1359 - protein, 11 aa, synthetic construct
  REGION 1..11: note = Synthetic
AQSTSSHTPAQ

SEQ ID NO: 1360 - protein, 11 aa, synthetic construct
  REGION 1..11: note = Synthetic
AQSTSSRTSAQ

SEQ ID NO: 1361 - protein, 11 aa, synthetic construct
  REGION 1..11: note = Synthetic
AQSTSSRTTAQ

SEQ ID NO: 1362 - protein, 11 aa, synthetic construct
  REGION 1..11: note = Synthetic
AQSTTSAPGAQ

SEQ ID NO: 1363 - protein, 11 aa, synthetic construct
  REGION 1..11: note = Synthetic
AQSTVTAVGAQ

SEQ ID NO: 1364 - protein, 11 aa, synthetic construct
  REGION 1..11: note = Synthetic
AQSVGELPFAQ

SEQ ID NO: 1365 - protein, 11 aa, synthetic construct
  REGION 1..11: note = Synthetic
AQSVPAKGSAQ

SEQ ID NO: 1366 - protein, 11 aa, synthetic construct
  REGION 1..11: note = Synthetic
AQSVRAGLNAQ

SEQ ID NO: 1367 - protein, 11 aa, synthetic construct
  REGION 1..11: note = Synthetic
AQSVRSPVTAQ

SEQ ID NO: 1368 - protein, 11 aa, synthetic construct
  REGION 1..11: note = Synthetic
AQSVRSSDYAQ

SEQ ID NO: 1369 - protein, 11 aa, synthetic construct
  REGION 1..11: note = Synthetic
AQSVTRTVAAQ

SEQ ID NO: 1370 - protein, 11 aa, synthetic construct
  REGION 1..11: note = Synthetic
AQSVYSHSSAQ

SEQ ID NO: 1371 - protein, 11 aa, synthetic construct
  REGION 1..11: note = Synthetic
AQSYAPQLLAQ

SEQ ID NO: 1372 - protein, 11 aa, synthetic construct
  REGION 1..11: note = Synthetic
AQSYSNRSVAQ

SEQ ID NO: 1373 - protein, 11 aa, synthetic construct
  REGION 1..11: note = Synthetic
AQTAAVRSGAQ

SEQ ID NO: 1374 - protein, 11 aa, synthetic construct
  REGION 1..11: note = Synthetic
AQTALKAVGAQ

SEQ ID NO: 1375 - protein, 11 aa, synthetic construct
  REGION 1..11: note = Synthetic
AQTANTRAEAQ

SEQ ID NO: 1376 - protein, 11 aa, synthetic construct
  REGION 1..11: note = Synthetic
AQTASQLSAAQ

SEQ ID NO: 1377 - protein, 11 aa, synthetic construct
  REGION 1..11: note = Synthetic
AQTDRITISAQ

SEQ ID NO: 1378 - protein, 11 aa, synthetic construct
  REGION 1..11: note = Synthetic
AQTDTRHATAQ

SEQ ID NO: 1379 - protein, 11 aa, synthetic construct
  REGION 1..11: note = Synthetic
AQTERNTASAQ

SEQ ID NO: 1380 - protein, 11 aa, synthetic construct
  REGION 1..11: note = Synthetic
AQTFHAVKEAQ

SEQ ID NO: 1381 - protein, 11 aa, synthetic construct
  REGION 1..11: note = Synthetic
AQTGSLAHGAQ

SEQ ID NO: 1382 - protein, 11 aa, synthetic construct
  REGION 1..11: note = Synthetic
AQTIGMRSAAQ

SEQ ID NO: 1383 - protein, 11 aa, synthetic construct
  REGION 1..11: note = Synthetic
AQTINKPNAAQ

SEQ ID NO: 1384 - protein, 11 aa, synthetic construct
  REGION 1..11: note = Synthetic
AQTISNSSGAQ

SEQ ID NO: 1385 - protein, 11 aa, synthetic construct
  REGION 1..11: note = Synthetic
AQTITRGTDAQ

SEQ ID NO: 1386 - protein, 11 aa, synthetic construct
  REGION 1..11: note = Synthetic
AQTLQNVNYAQ

SEQ ID NO: 1387 - protein, 11 aa, synthetic construct
  REGION 1..11: note = Synthetic
AQTLSSSREAQ

SEQ ID NO: 1388 - protein, 11 aa, synthetic construct
  REGION 1..11: note = Synthetic
AQTMGRNVSAQ

SEQ ID NO: 1389 - protein, 11 aa, synthetic construct
  REGION 1..11: note = Synthetic
AQTQGNRTDAQ

SEQ ID NO: 1390 - protein, 11 aa, synthetic construct
  REGION 1..11: note = Synthetic
AQTQHSTQSAQ

SEQ ID NO: 1391 - protein, 11 aa, synthetic construct
  REGION 1..11: note = Synthetic
AQTQSAYGSAQ

SEQ ID NO: 1392 - protein, 11 aa, synthetic construct
  REGION 1..11: note = Synthetic
AQTQSLRHDAQ

SEQ ID NO: 1393 - protein, 11 aa, synthetic construct
  REGION 1..11: note = Synthetic
AQTQTDRHSAQ

SEQ ID NO: 1394 - protein, 11 aa, synthetic construct
  REGION 1..11: note = Synthetic
AQTQTNIGRAQ

SEQ ID NO: 1395 - protein, 11 aa, synthetic construct
  REGION 1..11: note = Synthetic
AQTRHSSSDAQ

SEQ ID NO: 1396 - protein, 11 aa, synthetic construct
  REGION 1..11: note = Synthetic
AQTRSSSEAAQ

SEQ ID NO: 1397 - protein, 11 aa, synthetic construct
  REGION 1..11: note = Synthetic
AQTRSTIQEAQ

SEQ ID NO: 1398 - protein, 11 aa, synthetic construct
  REGION 1..11: note = Synthetic
AQTSATYSGAQ

SEQ ID NO: 1399 - protein, 11 aa, synthetic construct
  REGION 1..11: note = Synthetic
AQTSGTRHEAQ

SEQ ID NO: 1400 - protein, 11 aa, synthetic construct
  REGION 1..11: note = Synthetic
AQTSHAPNNAQ

SEQ ID NO: 1401 - protein, 11 aa, synthetic construct
  REGION 1..11: note = Synthetic
AQTSHSSSVAQ

SEQ ID NO: 1402 - protein, 11 aa, synthetic construct
  REGION 1..11: note = Synthetic
AQTSHVSSAAQ

SEQ ID NO: 1403 - protein, 11 aa, synthetic construct
  REGION 1..11: note = Synthetic
AQTSILRNEAQ

SEQ ID NO: 1404 - protein, 11 aa, synthetic construct
  REGION 1..11: note = Synthetic
AQTSNSSGQAQ

SEQ ID NO: 1405 - protein, 11 aa, synthetic construct
  REGION 1..11: note = Synthetic
AQTSNYGATAQ

SEQ ID NO: 1406 - protein, 11 aa, synthetic construct
  REGION 1..11: note = Synthetic
AQTSQSRVEAQ

SEQ ID NO: 1407 - protein, 11 aa, synthetic construct
  REGION 1..11: note = Synthetic
AQTSRANDTAQ

SEQ ID NO: 1408 - protein, 11 aa, synthetic construct
  REGION 1..11: note = Synthetic
AQTSRSNETAQ

SEQ ID NO: 1409 - protein, 11 aa, synthetic construct
  REGION 1..11: note = Synthetic
AQTSTPNKIAQ

SEQ ID NO: 1410 - protein, 11 aa, synthetic construct
  REGION 1..11: note = Synthetic
AQTSYSTTTAQ

SEQ ID NO: 1411 - protein, 11 aa, synthetic construct
  REGION 1..11: note = Synthetic
AQTTARNLEAQ

SEQ ID NO: 1412 - protein, 11 aa, synthetic construct
  REGION 1..11: note = Synthetic
AQTTGILRDAQ

SEQ ID NO: 1413 - protein, 11 aa, synthetic construct
  REGION 1..11: note = Synthetic
AQTTGTPRPAQ

SEQ ID NO: 1414 - protein, 11 aa, synthetic construct
  REGION 1..11: note = Synthetic
AQTTGYSQAAQ

SEQ ID NO: 1415 - protein, 11 aa, synthetic construct
  REGION 1..11: note = Synthetic
AQTTIRNAVAQ

SEQ ID NO: 1416 - protein, 11 aa, synthetic construct
  REGION 1..11: note = Synthetic
AQTTNITRTAQ

SEQ ID NO: 1417 - protein, 11 aa, synthetic construct
  REGION 1..11: note = Synthetic
AQTTSMGKVAQ

SEQ ID NO: 1418 - protein, 11 aa, synthetic construct
  REGION 1..11: note = Synthetic
AQTTSMSGHAQ

SEQ ID NO: 1419 - protein, 11 aa, synthetic construct
  REGION 1..11: note = Synthetic
AQTTSPKLPAQ

SEQ ID NO: 1420 - protein, 11 aa, synthetic construct
  REGION 1..11: note = Synthetic
AQTTSSSYNAQ

SEQ ID NO: 1421 - protein, 11 aa, synthetic construct
  REGION 1..11: note = Synthetic
AQTTSTHNAAQ

SEQ ID NO: 1422 - protein, 11 aa, synthetic construct
  REGION 1..11: note = Synthetic
AQTTSVRSTAQ

SEQ ID NO: 1423 - protein, 11 aa, synthetic construct
  REGION 1..11: note = Synthetic
AQTTTAGKVAQ

SEQ ID NO: 1424 - protein, 11 aa, synthetic construct
  REGION 1..11: note = Synthetic
AQTTTKFTPAQ

SEQ ID NO: 1425 - protein, 11 aa, synthetic construct
  REGION 1..11: note = Synthetic
AQTVITASGAQ

SEQ ID NO: 1426 - protein, 11 aa, synthetic construct
  REGION 1..11: note = Synthetic
AQTVQSHSTAQ

SEQ ID NO: 1427 - protein, 11 aa, synthetic construct
  REGION 1..11: note = Synthetic
AQTVSSGRPAQ

SEQ ID NO: 1428 - protein, 11 aa, synthetic construct
  REGION 1..11: note = Synthetic
AQTYNATRSAQ

SEQ ID NO: 1429 - protein, 11 aa, synthetic construct
  REGION 1..11: note = Synthetic
AQVAGRNVSAQ

SEQ ID NO: 1430 - protein, 11 aa, synthetic construct
  REGION 1..11: note = Synthetic
AQVAGTSQGAQ

SEQ ID NO: 1431 - protein, 11 aa, synthetic construct
  REGION 1..11: note = Synthetic
AQVAGYSRDAQ

SEQ ID NO: 1432 - protein, 11 aa, synthetic construct
  REGION 1..11: note = Synthetic
AQVATPSFLAQ

SEQ ID NO: 1433 - protein, 11 aa, synthetic construct
  REGION 1..11: note = Synthetic
AQVFTNRSAAQ

SEQ ID NO: 1434 - protein, 11 aa, synthetic construct
  REGION 1..11: note = Synthetic
AQVGHGSLPAQ

SEQ ID NO: 1435 - protein, 11 aa, synthetic construct
  REGION 1..11: note = Synthetic
AQVGSYSRDAQ

SEQ ID NO: 1436 - protein, 11 aa, synthetic construct
  REGION 1..11: note = Synthetic
AQVGTLSSSAQ

SEQ ID NO: 1437 - protein, 11 aa, synthetic construct
  REGION 1..11: note = Synthetic
AQVGVNVHGAQ

SEQ ID NO: 1438 - protein, 11 aa, synthetic construct
  REGION 1..11: note = Synthetic
AQVIRGLEQAQ

SEQ ID NO: 1439 - protein, 11 aa, synthetic construct
  REGION 1..11: note = Synthetic
AQVISRDSSAQ

SEQ ID NO: 1440 - protein, 11 aa, synthetic construct
  REGION 1..11: note = Synthetic
AQVISSHHSAQ

SEQ ID NO: 1441 - protein, 11 aa, synthetic construct
  REGION 1..11: note = Synthetic
AQVKANQATAQ

SEQ ID NO: 1442 - protein, 11 aa, synthetic construct
  REGION 1..11: note = Synthetic
AQVLHASMSAQ

SEQ ID NO: 1443 - protein, 11 aa, synthetic construct
  REGION 1..11: note = Synthetic
AQVLHGSSYAQ

SEQ ID NO: 1444 - protein, 11 aa, synthetic construct
  REGION 1..11: note = Synthetic
AQVLRSNVMAQ

SEQ ID NO: 1445 - protein, 11 aa, synthetic construct
  REGION 1..11: note = Synthetic
AQVNLPVLLAQ

SEQ ID NO: 1446 - protein, 11 aa, synthetic construct
  REGION 1..11: note = Synthetic
AQVPGHSGLAQ

SEQ ID NO: 1447 - protein, 11 aa, synthetic construct
  REGION 1..11: note = Synthetic
AQVPGQSSLAQ

SEQ ID NO: 1448 - protein, 11 aa, synthetic construct
  REGION 1..11: note = Synthetic
AQVPRSSTDAQ

SEQ ID NO: 1449 - protein, 11 aa, synthetic construct
  REGION 1..11: note = Synthetic
AQVPRTGLTAQ

SEQ ID NO: 1450 - protein, 11 aa, synthetic construct
  REGION 1..11: note = Synthetic
AQVPTSHSTAQ

SEQ ID NO: 1451 - protein, 11 aa, synthetic construct
  REGION 1..11: note = Synthetic
AQVQSHGVNAQ

SEQ ID NO: 1452 - protein, 11 aa, synthetic construct
  REGION 1..11: note = Synthetic
AQVQYGGLTAQ

SEQ ID NO: 1453 - protein, 11 aa, synthetic construct
  REGION 1..11: note = Synthetic
AQVRGLYSDAQ

SEQ ID NO: 1454 - protein, 11 aa, synthetic construct
  REGION 1..11: note = Synthetic
AQVRHSGMEAQ

SEQ ID NO: 1455 - protein, 11 aa, synthetic construct
  REGION 1..11: note = Synthetic
AQVRPPNPSAQ

SEQ ID NO: 1456 - protein, 11 aa, synthetic construct
  REGION 1..11: note = Synthetic
AQVRSTLTDAQ

SEQ ID NO: 1457 - protein, 11 aa, synthetic construct
  REGION 1..11: note = Synthetic
AQVRSVGLDAQ

SEQ ID NO: 1458 - protein, 11 aa, synthetic construct
  REGION 1..11: note = Synthetic
AQVRTPLFAAQ

SEQ ID NO: 1459 - protein, 11 aa, synthetic construct
  REGION 1..11: note = Synthetic
AQVRTSYSDAQ

SEQ ID NO: 1460 - protein, 11 aa, synthetic construct
  REGION 1..11: note = Synthetic
AQVSGIRGDAQ

SEQ ID NO: 1461 - protein, 11 aa, synthetic construct
  REGION 1..11: note = Synthetic
AQVSGRSLSAQ

SEQ ID NO: 1462 - protein, 11 aa, synthetic construct
  REGION 1..11: note = Synthetic
AQVSNSYLGAQ

SEQ ID NO: 1463 - protein, 11 aa, synthetic construct
  REGION 1..11: note = Synthetic
AQVSNTYSGAQ

SEQ ID NO: 1464 - protein, 11 aa, synthetic construct
  REGION 1..11: note = Synthetic
AQVSRSESAAQ

SEQ ID NO: 1465 - protein, 11 aa, synthetic construct
  REGION 1..11: note = Synthetic
AQVSSYPNSAQ

SEQ ID NO: 1466 - protein, 11 aa, synthetic construct
  REGION 1..11: note = Synthetic
AQVTGLRGIAQ

SEQ ID NO: 1467 - protein, 11 aa, synthetic construct
  REGION 1..11: note = Synthetic
AQVTHASLSAQ

SEQ ID NO: 1468 - protein, 11 aa, synthetic construct
  REGION 1..11: note = Synthetic
AQVTQLGRSAQ

SEQ ID NO: 1469 - protein, 11 aa, synthetic construct
  REGION 1..11: note = Synthetic
AQVTRSAGEAQ

SEQ ID NO: 1470 - protein, 11 aa, synthetic construct
  REGION 1..11: note = Synthetic
AQVTSKNDLAQ

SEQ ID NO: 1471 - protein, 11 aa, synthetic construct
  REGION 1..11: note = Synthetic
AQVTTKTQLAQ

SEQ ID NO: 1472 - protein, 11 aa, synthetic construct
  REGION 1..11: note = Synthetic
AQVVANTSRAQ

SEQ ID NO: 1473 - protein, 11 aa, synthetic construct
  REGION 1..11: note = Synthetic
AQVVGSNREAQ

SEQ ID NO: 1474 - protein, 11 aa, synthetic construct
  REGION 1..11: note = Synthetic
AQVVRSGNEAQ

SEQ ID NO: 1475 - protein, 11 aa, synthetic construct
  REGION 1..11: note = Synthetic
AQVVRSVNDAQ

SEQ ID NO: 1476 - protein, 11 aa, synthetic construct
  REGION 1..11: note = Synthetic
AQVVRTPNSAQ

SEQ ID NO: 1477 - protein, 11 aa, synthetic construct
  REGION 1..11: note = Synthetic
AQWGSHPTGAQ

SEQ ID NO: 1478 - protein, 11 aa, synthetic construct
  REGION 1..11: note = Synthetic
AQWLKSVSSAQ

SEQ ID NO: 1479 - protein, 11 aa, synthetic construct
  REGION 1..11: note = Synthetic
AQWRHTSAEAQ

SEQ ID NO: 1480 - protein, 11 aa, synthetic construct
  REGION 1..11: note = Synthetic
AQYAGNSGNAQ

SEQ ID NO: 1481 - protein, 11 aa, synthetic construct
  REGION 1..11: note = Synthetic
AQYASSSNNAQ

SEQ ID NO: 1482 - protein, 11 aa, synthetic construct
  REGION 1..11: note = Synthetic
AQYERNHNAAQ

SEQ ID NO: 1483 - protein, 11 aa, synthetic construct
  REGION 1..11: note = Synthetic
AQYKSSLSEAQ

SEQ ID NO: 1484 - protein, 11 aa, synthetic construct
  REGION 1..11: note = Synthetic
AQYLHGLQQAQ

SEQ ID NO: 1485 - protein, 11 aa, synthetic construct
  REGION 1..11: note = Synthetic
AQYNQIRGDAQ

SEQ ID NO: 1486 - protein, 11 aa, synthetic construct
  REGION 1..11: note = Synthetic
AQYNSSNFGAQ

SEQ ID NO: 1487 - protein, 11 aa, synthetic construct
  REGION 1..11: note = Synthetic
AQYPGNRMEAQ

SEQ ID NO: 1488 - protein, 11 aa, synthetic construct
  REGION 1..11: note = Synthetic
AQYRNTVQDAQ

SEQ ID NO: 1489 - protein, 11 aa, synthetic construct
  REGION 1..11: note = Synthetic
AQYRQSSSDAQ

SEQ ID NO: 1490 - protein, 11 aa, synthetic construct
  REGION 1..11: note = Synthetic
AQYRSGTQEAQ

SEQ ID NO: 1491 - protein, 11 aa, synthetic construct
  REGION 1..11: note = Synthetic
AQYTGSISVAQ

SEQ ID NO: 1492 - protein, 11 aa, synthetic construct
  REGION 1..11: note = Synthetic
AQYTSSVTGAQ

SEQ ID NO: 1493 - protein, 11 aa, synthetic construct
  REGION 1..11: note = Synthetic
AQYVASSPMAQ

SEQ ID NO: 1494 - protein, 11 aa, synthetic construct
  REGION 1..11: note = Synthetic
AQYVGSGTAAQ

SEQ ID NO: 1495 - protein, 11 aa, synthetic construct
  REGION 1..11: note = Synthetic
AQYVHSNNAAQ

SEQ ID NO: 1496 - protein, 11 aa, synthetic construct
  REGION 1..11: note = Synthetic
AQYYSSSGGAQ